(19) United States
(12) Patent Application Publication
(10) Pub. No.: US 2020/0325229 A1
(43) Pub. Date: Oct. 15, 2020

(54) METHODS FOR TREATING LYMPHOMAS

(71) Applicant: SINGAPORE HEALTH SERVICES PTE LTD, Singapore (SG)

(72) Inventors: Choon Kiat ONG, Singapore (SG); Soon Thye LIM, Singapore (SG); Jing Quan LIM, Singapore (SG)

(21) Appl. No.: 16/754,068

(22) PCT Filed: Oct. 10, 2018
(86) PCT No.: PCT/SG2018/050509

§ 371 Date: Apr. 6, 2020

(30) Foreign Application Priority Data
Oct. 6, 2017 (SG) 10201708262R

(51) Int. Cl. C07K 16/28; C12Q 1/6886; A61P 35/00

(52) U.S. Cl. CPC C07K 16/2818; A61K 2039/505; A61P 35/00; C12Q 1/6886

(57) ABSTRACT

Disclosed herein are methods for treating natural killer/T-cell lymphoma in a subject, comprising administering to the subject a PD-1/CD279 inhibitor, a PD-L1/CD274 inhibitor, or a combination thereof. Also disclosed herein are methods of determining response of a subject suffering from natural killer/T-cell lymphoma to pembrolizumab treatment, comprising detecting the presence or absence of at least one JAK3 activating mutation or at least one PD-L1 structural rearrangement.

### CROSS-REFERENCE TO RELATED APPLICATIONS

This application claims the benefit of priority of SG provisional application No. 10201708262R, filed 6 Oct. 2017, the contents of it being hereby incorporated by reference in its entirety for all purposes.

### FIELD OF THE INVENTION

The present invention relates generally to the field of molecular biology. In particular, the present invention relates to the use of biomarkers for the detection and treatment of cancer.

### BACKGROUND OF THE INVENTION

Natural-killer/T cell lymphoma (NKTL) is an uncommon and aggressive malignancy with a predilection for Asian, Mexican and South American populations. With the exception of Japan, it is the most common mature T-cell lymphoma in Asia. Neoplastic cells are invariably infected by the Epstein Barr virus (EBV) and are characterized by a cytotoxic phenotype.

Immune checkpoint inhibitors have changed the landscape for treatment of many cancers, including some hematologic malignancies. Investigations on several solid tumours, including non-small-cell lung carcinoma, melanoma and bladder cancer, have generally concluded that immunohistochemistry (IHC) PD-L1 positivity coincides with greater likelihood of response to PD-1/PD-L1 blockade. However, there was also a lower but definite response rate in patients with PD-L1-negative tumours. These observations highlight the many pitfalls of adopting PD-L1 immunohistochemistry, based on a single tumour specimen per patient, as an absolute selection criterion for PD-1 blockade therapy.

Extranodal natural killer/T-cell nasal-type lymphoma (ENKL) is an uncommon and aggressive malignancy with a predilection for Asian, Mexican and South American populations. To date, there is no targeted therapy available for the treatment of ENKL. As anthracycline-based regimens with ENKL are associated with dismal results, L-asparaginase-based regimens, like the SMILE (dexamethasone, methotrexate, ifosfamide. L-asparaginase, etoposide) regimen, have significantly improved clinical outcomes, especially for patients with disseminated disease. However, SMILE or SMILE-like regimens still fail in up to 40 to 50% of the cases and the toxicities associated with SMILE also preclude its use in older patients.

Furthermore, there is still no FDA approved targeted regime to manage natural-killer/T-cell lymphoma (NKTL) as the disease is uncommon, and made it challenging to identify biomarker of response to therapy. Thus, there is an unmet need for methods of identifying natural-killer/T-cell lymphoma and for methods of treating the same.

### SUMMARY

In one aspect, the present invention refers to a method of treating natural killer/T-cell lymphoma in a subject, the method comprising administering to a subject a therapeutically effective amount of pembrolizumab, wherein the subject is characterised by the presence of at least one JAK3-activating mutation or at least one PD-L1 structural rearrangement.

In another aspect, the present invention refers to a method of determining response of a subject suffering from natural killer/T-cell lymphoma to pembrolizumab treatment, the method comprising obtaining a sample from the subject; detecting the presence or absence of at least one JAK3 activating mutation or at least one PD-L1 structural rearrangement; wherein the presence of at least one JAK activating mutation or at least one PD-L1 structural rearrangement indicates that the subject will respond to pembrolizumab treatment.

In yet another aspect, the present invention refers to a kit for detecting the presence or absence of at least one JAK3 activating mutation or at least one PD-L1 structural rearrangement, the kit comprising a detection agent, and at least one pair of primers; wherein the primers enrich for the genomic regions of the JAK3 and PD-L1 genes.

In a further aspect, the present invention refers to a kit for detecting the presence or absence of at least one JAK3 activating mutation or at least one PD-L1 structural rearrangement for next-generation sequencing.

In another aspect, the present invention refers to a kit as disclosed herein for use according to the method as disclosed herein.

### BRIEF DESCRIPTION OF THE DRAWINGS

The invention will be better understood with reference to the detailed description when considered in conjunction with the non-limiting examples and the accompanying drawings, in which:

FIG. 1 shows genomic profiles of 11 pre-treated natural-killer/T-cell lymphoma tumours from patients who were subsequently treated with pembrolizumab. FIG. 1A shows a staircase plot of recurrent and mutually exclusive non-silent genomic alterations found in the 11 pairs of NKTL-normal whole-genome sequencing data. The top of the staircase plot denotes the number of non-silent mutations. FIG. 1B shows the schematics of the PD-L1 structural rearrangements that were validated in this study. FIG. 1C shows the positron emission tomography-computed tomography frontal and side scans of an NKTL1 patient, who had achieved complete response from pembrolizumab, before and after treated with pembrolizumab.

FIG. 2 shows the timelines of treatment for the eleven extranodal natural killer/T-cell lymphoma patients, who were administered pembrolizumab, after failing multiple lines of treatment.

FIG. 3 refers to recurrent somatic mutated genes in the 11 pembrolizumab-treated patients' initial tumours. Precedence of ordering, from top to bottom gene, is by recurrence and mutual exclusivity of genes within the cohort of patients who achieved complete response from pembrolizumab therapy. MAF of 1%, from wAnnovar's 1 k genome and ExAC databases, is used as cut-off.

FIG. 4 shows the validation of PD-L rearrangements and JAK3-activating mutation identified in natural-killer/t-cell lymphoma patients with complete response to pembrolizumab. Sanger sequencing was used to confirm the breakpoints of each PD-L1 structural rearrangement and the JAK3 mutations identified by whole-genome sequencing. The gene structure of the wild-type (WT) PD-L1 is shown at the top as reference. The breakpoints implicating each of the predicted rearranged PD-L1 are shown below. White arrows represent introns and the orientation of transcription. All tumours are biopsies before pembrolizumab has been administered. NKTL1, NKTL26, NKTL28 and NKTL31 harboured rearranged PD-L1. NKTL29 and NKTL30 were validated to harbour the G>A mutations that translated to JAK3 p.A573V.

FIG. 5 shows the schematics of the tandem duplication disrupting the 3′UTR of PD-L1 in NKTL26 inferred from whole genome sequencing data. The wild type region within 9p24.1 has been divided into three blocks (Q, R and S), each of which is shown in a different colour. The boundaries between Q-R and R-S denote the breakpoints of the tandem duplication. The rearrangement is heterozygous and the schematics display both the wild-type alleles in the matching-normal sample and, wildtype and mutant alleles in the tumour. The total copy number of PD-L1 in the tumour is three; the mutant allele has a PD-L1 with a disrupted 3′UTR. Wild type allele contains Q+R+S and the mutant allele contains Q+R1+R+S. When the genomic region of R1+R+S of the mutant allele is transcribed, a 3′UTR disrupted PD-L1 and wild type PD-L1 will be transcribed from R1 and R, respectively. The two dotted lines denote the boundaries of the tandem duplication on a wild type genomic scale.

FIG. 6 refers to clonality cluster plots from SciClone. SciClone was recommended to analyze only single nucleotide variants called from genomic regions of copy-2 number and without loss of heterozygosity (LOH). Hence, CANVAS was used to obtain copy number and LOH information. As only heterozygous mutations were analysed, the founding clone for a tumour of 100% cancer cellular fraction would at most yield a cluster that resides around the 50% mark of the cluster plot from SciClone.

FIG. 7 illustrates frequent somatic PD-L structural rearrangement (SR) uncovered by whole genome sequencing (WGS) data from 32 pairs of tumor-normal extranodal natural killer/T-cell lymphoma samples FIG. 7A shows the staircase plot for the recurrent mutated genes in an extended cohort of 32 extranodal natural killer/T-cell lymphoma untreated samples. The type of mutations affecting each gene is appended to the bottom of the staircase plot. FIG. 7B refers to a 3-track circos representation of the somatic SR detected in the fresh-frozen WGS samples. The outermost track represents the main human chromosomes from the hs37 reference genome. The middle track is a histogram that depicts the number of unique samples, from minimum of zero (inner track) to a maximum of eight (outer track), which have SR breakpoints in the corresponding genomic region. The inner track has black arcs, which each is an SR that disrupted the 3′UTR of PD-L1. FIG. 7C shows the schematics of the PD-L structural rearrangements that were validated in the cohort of 32 untreated samples.

FIG. 8 refers to the Sanger validation of PD-L rearrangements identified in within the cohort of 32 untreated NKTL samples. Sanger sequencing was used to confirm the breakpoints (in broken lines) of each PD-L1 structural rearrangement identified by whole-genome sequencing. The gene structure of the wild-type (WT) PD-L1 is shown at the top as reference. The chromatogram of the Sanger sequenced SR accompanies the schematic drawing of the rearranged PD-L. White arrows represent introns and the orientation of transcription. All tumours are biopsies before pembrolizumab has been administered. NKTL6 harbours a rearrangement with combined 3′UTR deletion and insertion of an upstream 73 bp inverted intronic sequence (complex*). NKTL1, NKTL26, NKTL28 and NKTL31 are samples from the Pembrolizumab-treatment cohort. NKTL4, NKTL6, NKTL11, NKTL15, NKTL16 and NKTL17 are samples in the prevalence untreated cohort. All tumours are initial biopsies before any treatment has been administered.

FIG. 9 illustrates aberrant fusion transcripts of PD-L1. Panel ‘NKTL16’ shows the genomic and transcriptomic structures of PD-L1 translocation to chromosome 6 in sample NKTL16. Panel ‘NKTL6’ shows the complex intra-chromosomal rearrangement in sample NKTL6 where the 3′UTR deletion was accompanied by insertion of an upstream 73 bp inverted intronic sequence. Panel ‘NKTL15’ shows the tandem duplication in sample NKTL15. Panel ‘NKTL4’ shows the intra-chromosomal deletion in sample NKTL4. Panel ‘NKTL17’ also shows the intra-chromosomal deletion in sample NKTL17. Broken lines and arrows indicate the breakpoints and fusion orientation, respectively. Q, R and S blocks symbolize the transcript blocks. Triangles represent the orientation of transcription and the polyadenylation (polyA) signals are shown by black arrows. Aberrant and wild-type PD-L mRNA transcript levels were obtained from whole-transcriptome sequencing data and are presented in dark and light gray colors, respectively. Accompanying copy number (CN) alteration is also shown for the tandem duplication event. Breakpoint validation was done with Sanger sequencing on the chimeric cDNA and the chromatograms are displayed for each case.

### DETAILED DESCRIPTION OF THE PRESENT INVENTION

In recent years, immune checkpoint (ICP) inhibitors have shown promising objective response rates (ORR) in the treatment of many malignancies. Of note, one result shows 80% objective response rates from the use of programmed death-1 (PD-1 or CD279) inhibitors in relapsed or refractory (RR) Hodgkin lymphoma (HL). Currently, clinical studies involving non-small-cell lung carcinoma, melanoma and bladder cancer have generally concluded that immunohistochemistry (IHC) positivity of programmed death-ligand 1 (PD-L1) coincides with greater likelihood of response to PD-1/PD-L1 blockade. Intriguingly, there was also a lower but definite response rate in patients with PD-L1-negative tumours. These observations suggest that more information could be harnessed from the tumours and augment the current de facto criteria of selecting patients for PD-1 blockade therapy.

The inventors have identified recurrent genetic alterations in relapsed or refractory natural killer/T-cell lymphoma (RR NKTL) patients who have achieved complete response (CR) with programmed cell death 1 (PD-1) blockade therapy.

With the advancements in sequencing technologies, recurring somatic mutations altering the JAK-STAT pathway, epigenetic modifiers, DDX3X gene and germline genetic predisposition in the HLA-DPB1 gene have been found in natural killer/T-cell lymphoma (NKTL) patients, but none of these studies have used whole genome sequencing (WGS) techniques. In order to explore the natural killer/T-cell lymphoma genomes in a high sequencing throughput and genome-wide fashion for targetable genomic alterations, whole genome sequencing data was employed to study the somatic alterations of 11 pre-treated natural killer/T-cell lymphoma tumours that have subsequent clinical response data to pembrolizumab treatment.

Through whole-genome sequencing of paired natural killer/T-cell lymphoma (NKTL) tumour-normal samples, it was shown that a somatic breakpoint-cluster is present within the programmed cell death ligand 1 (PD-L1/CD274) gene that is highly recurrent in 36% (4 of 11) of the tumours. These structural rearrangements (SRs) are validated to disrupt the 3′ untranslated region (UTR) of the PD-L1 gene, which result in the aberrant expression of PD-L1 chimeric transcripts.

In one example, among 11 individuals with relapsed or refractory natural killer/T-cell lymphoma (NKTL) were treated with pembrolizumab, PD-L1 3′UTR structural rearrangements were found in all four responders but absent in the four non-responders. Without being bound by theory, it was thought that PD-L1 3′UTR structural rearrangement was associated with response to PD-1 blockade and reduced M2-macrophage signature, thereby allowing the use of PD-1 blockade therapy for PD-L-rearranged natural killer/T-cell lymphomas and, in turn, improving treatment outcome for these patients.

Disclosed herein is a method of treating natural killer/T-cell lymphoma in a subject, the method comprising administering to a subject a therapeutically effective amount of pembrolizumab, wherein the molecular genomic profile of the subject is characterised by the presence of at least one PD-L1 structural rearrangement. In one example, there is disclosed a method of treating natural killer/T-cell lymphoma in a subject, the method comprising administering to a subject a therapeutically effective amount of pembrolizumab, wherein the subject is characterised by the presence of at least one JAK3-activating mutation or at least one PD-L1 structural rearrangement.

Thus, in one example, the structural rearrangement disrupts the 3′ untranslated region (3′ UTR) of the PD-L1 gene. In another example, the PD-L1 structural rearrangement is a mutation in the PD-L1 gene. In yet another example, the mutation in the PD-L1 gene disrupts the 3′ UTR of the PD-L1 gene.

In one example, the JAK3-activating mutation is, but is not limited to, any one or more of the following mutations: M511I, A572V, A573V, R657Q, V722I, V674A, L857P, R403H, Q501H, E958K. In another example, the JAK3-activating mutation is a single-nucleotide substitution (p.A572V or p.A573V) in the JAK3 gene (JAK3 RefSeq Gene ID: NM_000215). In other words, in one example, the JAK3 activating mutation is A572V. The terms “JAK3-activating” mutation” and “JAK3 mutation” are considered to be interchangeable.

As used herein, the term “mutation” refers to permanent alteration of the nucleotide sequence of the genome of an organism or a genetic element. The mutation can be, but is not limited to, insertions, deletions, substitutions, translocations, inversions, micro-inversions, duplications, tandem repeats, breakpoint(s) (mutations), and combinations thereof.

As used herein, the term “structural rearrangement” refers to one or more mutations that result in a change in the overall structure of the nucleic acid sequence of interest. A “structural rearrangement” spans across a genomic region and the boundaries of this mutation are known as breakpoints. For example, in the event that a breakpoint resides in a gene, the mutations as disclosed herein result in a change in the structure of said gene. Such structural rearrangements can also refer to changes in the chromosomal structure that encompasses the gene or nucleic acid sequence of interest. Thus, in one example, the mutation is a micro-inversion, inversion, translocation, tandem repeat, or a breakpoint (mutation), or combinations thereof.

As used herein, the term “inversion” refers to an inversion of a nucleic acid sequence within a specific sequence, whereby the sequence is excised and inserted in the reverse orientation compared to the orientation it was in before. In other words, the nucleic acid sequence of interest is reversed end to end as the result of a mutation. The term “micro-inversion” refers to nucleic acid sequences from 50 to 1000 bp (base pairs) in length. In one example, the mutation is a micro-inversion of between 150 to 250 bp in length. In one example, the mutation is a micro-inversion of between 200 to 210 bp in length. In another example, the mutation is a micro-inversion of about 206 bp in length.

As used herein, the term “relapse” or “recidivism” refers to a recurrence of a past condition, such as, for example, a medical condition. There are medical conditions known for having extended relapse periods (for example, malaria). In the present context, the term “relapse” refers to the scenario where a medical condition previously existed (for example, the presence of a particular disease), which had been treated or was no longer present in a subject, which has now reoccurred or re-surfaced in the subject.

As used herein, the term “refractory” refers to a disease or condition which does not respond to any attempted forms of treatment. For example, a cancer is said to be refractory when it does not respond to (or is resistant to) cancer treatment. Refractory cancer is also known as resistant cancer.

Thus, in one example, the natural killer/T-cell lymphoma described herein is a relapsed and/or refractory natural killer/T-cell lymphoma. In one example, the natural killer/T-cell lymphoma is a relapsed natural killer/T-cell lymphoma. In another example, the natural killer/T-cell lymphoma is a refractory natural killer/T-cell lymphoma.

### Response to Pembrolizumab in Relapsed or Refractory Natural Killer/T-Cell Lymphoma (NKTL) Patients

Eleven natural killer/T-cell lymphoma patients from Singapore, China and Hong Kong who were relapsed or refractory (RR) to L-asparaginase containing chemotherapy regimens, after a median of two (range between 1 to 5 lines of treatment) lines of treatments, were included into this study (Table 1). These eleven pembrolizumab-treated patients had a median age of 42 years old at diagnosis (range between 27 to 66 years of age) and a median follow-up time of eleven months (range between 2 to 25 months) since treated with pembrolizumab. Sixty-four percent (64%; 7 of 11 cases) of the patients achieved complete responses (CR) while 36% (4 of 11 cases) of the patients had progressive disease (PD). Two patients (NKTL26 & NKTL31) remained in remission from pembrolizumab for more than two years, which is considered to be a rare occurrence in relapsed or refractory natural killer/T-cell lymphoma (RR NKTL). The most recent pembrolizumab-treated case (NKTL28) achieved ongoing remission for at least 6 months. The median duration of response to pembrolizumab (for responding patients) was 14 months.

TABLE 1
Details of the eleven natural killer/T-cell lymphoma patients from Singapore, China and Hong Kong who were relapsed or refractory (RR)
to L-asparaginase containing chemotherapy regimens, after a median of two (range between 1 to 5 lines of treatment) lines of treatments

| | | | | | | | | | | | | | Pembrolizumab | |
|---|---|---|---|---|---|---|---|---|---|---|---|---|---|---|
| | | | | | | | | Treatments prior to Pembrolizumab | | | | | treatment | |
| | | | Age, | | | OS, | PFS, | CTx | | | PD-L1+, % | | | DOR¹, |
| | Case | Sex | yr | Stage | ECOG | mth | mth | (cycles) | RT | TP | (H-score) | | Outcome | mth |
| Complete | NKTL1 | M | 49 | IV | 1 | 49+ | 19 | GELOX (4), | Nil | Nil | 100% | (250) | CR: | 20+ |
| Responders | | | | | | | | SMILE (5), | | | | | PET/CT | |
| (CR) | | | | | | | | Romidepsin + | | | | | EBV DNA: | |
| | | | | | | | | Bortezomib, | | | | | negative | |
| | | | | | | | | BV + Benda, | | | | | then became | |
| | | | | | | | | Lenalidomide + | | | | | positive and | |
| | | | | | | | | Dara | | | | | remained | |
| | | | | | | | | | | | | | stable | |
| | NKTL26 | M | 32 | I | 1 | 44+ | 2 | SMILE (2), | Yes | Nil | 40% | (35) | CR: | 24+ |
| | | | | | | | | Vinc + | | | | | PET/CT | |
| | | | | | | | | DXM + | | | | | EBV DNA: | |
| | | | | | | | | Lasp (1), | | | | | ND | |
| | | | | | | | | GELOX (6) | | | | | | |
| | NKTL28 | M | 46 | IV | 3 | 9+ | 0 | SMILE (2), | Nil | Nil | 70% | (190) | CR: | 6+ |
| | | | | | | | | P-GEMOX (1) | | | | | PET/CT | |
| | | | | | | | | | | | | | EBV DNA: | |
| | | | | | | | | | | | | | negative | |
| | NKTL29 | M | 48 | I | 0 | 13+ | 4 | Ifos + | Nil | Nil | 6% | (7) | CR: | 9+ |
| | | | | | | | | MTX + | | | | | PET/CT | |
| | | | | | | | | VP-16 + | | | | | EBV DNA: | |
| | | | | | | | | DXM + | | | | | negative | |
| | | | | | | | | Pasp (4) | | | | | | |
| | NKTL30 | M | 38 | IV | 3 | 19+ | 6 | SMILE (5) | Nil | Nil | 60% | (120) | CR: | 11+ |
| | | | | | | | | | | | | | PET/CT | |
| | | | | | | | | | | | | | EBV DNA: | |
| | | | | | | | | | | | | | negative | |
| | NKTL31 | M | 27 | IV | 0 | 67+ | 17 | Lasp + | Nil | Auto-HSCT | 20% | (20) | CR: CT & | 24+ |
| | | | | | | | | DXM + | | with | | | MRI | |
| | | | | | | | | Vinc + Ara | | BEAM + | | | EBV DNA: | |
| | | | | | | | | C (4), | | Thalidomide | | | negative | |
| | | | | | | | | CHOP (2), | | (mainte- | | | | |
| | | | | | | | | P-GEMOX (2), | | nance) | | | | |
| | | | | | | | | DXM + | | | | | | |
| | | | | | | | | Pasp + | | | | | | |
| | | | | | | | | mitoxantrone + | | | | | | |
| | | | | | | | | VP-16 (4) | | | | | | |
| | | | | | | | | P-GEMOX + | | | | | | |
| | | | | | | | | VP-16 (2) | | | | | | |
| | NKTL43 | M | 29 | IV | 2 | 116 | 73 | m-BACOD (4), | Yes | Nil | 90% | (190) | CR: | 14 |
| | | | | | | | | PIGLET (5), | | | | | PET/CT | |
| | | | | | | | | SMILE (3) | | | | | EBV DNA: | |
| | | | | | | | | | | | | | negative | |
| | | | | | | | | | | | | | Patient | |
| | | | | | | | | | | | | | subsequently | |
| | | | | | | | | | | | | | underwent | |
| | | | | | | | | | | | | | MUD BMT | |
| | | | | | | | | | | | | | and died | |
| | | | | | | | | | | | | | from | |
| | | | | | | | | | | | | | GVHD. | |
| Non-CR | NKTL25 | M | 30 | IV | 0 | 14 | 10 | SMILE (6), | Yes | Allo-HSCT | 72% | (126) | PD: DOD | NA |
| | | | | | | | | GEMOX (1) | | | | | | |
| | NKTL27 | M | 59 | IV | 0 | 19 | 2 | SMILE (3), | Nil | Nil | 50% | (85) | PD: DOD | NA |
| | | | | | | | | GIFOX (4) | | | | | | |
| | NKTL44 | M | 66 | IV | 1 | 37 | 21 | SIMPLE (6) | Nil | Nil | 90% | (170) | PD: DOD | NA |
| | NKTL45 | M | 42 | IV | 1 | 94 | 87 | SMILE (6), | Nil | Allo-HSCT | 65% | (70) | PD: DOD | NA |
| | | | | | | | | GEMOX (1) | | | | | | |

¹DOR: Durability of response as of January 2018; + indicates ongoing survival
BV, bretuximab vedotin;
Benda, bendamustine;
Dara, daratumumab;
Vinc, vincristine;
DXM, dexamethasone;
Lasp, L-asparaginase;
Ifos, ifosfamide;
MTX, methotrexate;
VP-16, etoposide;
Pasp, Peg-Lasparaginase;
AraC, cytarabine;
ND, not done;
RT, radiotherapy;
TP, transplant

Thus, in one example, the subject had previously not responded to SMILE (dexamethasone, methotrexate, ifosfaminde, L-asparaginase and etoposide) therapy. In another example, the subject had previously responded to SMILE. That is to say that the subject had previously responses to SMILE therapy, however, that the disease has re-occurred or relapsed. In another example, the subject had been previously treated with any one or more of the compounds dexamethasone, methotrexate, ifosfaminde, L-asparaginase or etoposide, or combinations thereof.

PD-L1 Positivity could not Stratify Response to Pembrolizumab in Natural Killer/T-Cell Lymphoma (NKTL) Patients

To verify if PD-L1 positivity in natural killer/T-cell lymphoma (NKTL) tumours could predict response to pembrolizumab, the positivity of PD-L1 in all 11 pre-treated NKTL tumours was determined using immunohistochemistry (IHC). The same pathologist assessed PD-L1 positivity in all the tumours in this study to ensure consistency Table 2). The PD-L1 positivity in the tumour cells varied greatly in both patients who achieved complete responses and progressive disease. PD-L1 positivity in the pre-treated tumours of the patients with complete responses ranged from 6% to 100% while the PD-L1 staining intensity among patients with progressive disease ranged from 35% to 90%. Hence PD-L1 staining intensity could not differentiate between patients who achieved complete response and those who had progressive disease. Interestingly, NKTL29 had only 6% of tumour cells stained positive for PD-L1 but achieved complete response from pembrolizumab. Apart from this PD-Lllow complete response case, all four progressive disease cases were strongly stained for PD-L1, with an average of 69% (range: 50% to 90%) tumour cells stained positively for PD-L1. This is concordant with clinical trials reporting that anti-tumor activity from PD-1 blockade therapy was also observed in melanoma and non-small cell lung carcinoma patients with low baseline PD-L1 positivity. In contrast, the method disclosed herein shows that some patients with low PD-L1 positivity may have good responses to PD-1 blockade. In summary and without being bound by theory, this goes against what is known in the art, as based on the immunohistochemistry staining as shown in the art, subjects that achieved complete response to PD-1 blockade should significantly associate with higher PD-L1 positivity in their tumours than of those who did not.

TABLE 2
Membraneous PD-L1 immunohistochemical staining grade, PD-L1 H-score and PD-L1 positivity cells in
the pretreated NKTL tumours of the 11 patients who were subsequently treated with pembrolizumab.

| | | | PD-L1 | | | | |
|---|---|---|---|---|---|---|---|
| | | | positivity | PD-L1 | PD-L1 | PD-L1 | |
| | Response to | PD-L1 | %, strongest | lymphocytes | lymphocytes | lymphocytes | H-score |
| Sample ID | pembrolizumab | Rearranged | stain grade | 1+^{a} | 2+^{a} | 3+^{a} | for PD-L1 |
| NKTL1 | CR | Yes | 100%, 3+ | 0% | 50% | 50% | 250 |
| NKTL26 | CR | Yes | 35%, 2+ | 30% | 5% | 0% | 40 |
| NKTL28 | CR | Yes | 70%, 3+ | 0% | 20% | 50% | 190 |
| NKTL31 | CR | Yes | 20%, 1+ | 20% | 0% | 0% | 20 |
| NKTL29 | CR | No | 6%, 2+ | 5% | 1% | 0% | 7 |
| NKTL30 | CR | No | 60%, 3+ | 10% | 40% | 10% | 120 |
| NKTL43 | CR | No | 90%, 3+ | 20% | 40% | 30% | 190 |
| NKTL25 | PD | No | 72%, 3+ | 20% | 50% | 2% | 126 |
| NKTL27 | PD | No | 50%, 3+ | 20% | 25% | 5% | 85 |
| NKTL44 | PD | No | 90%, 3+ | 20% | 60% | 10% | 170 |
| NKTL45 | PD | No | 65%, 2+ | 60% | 5% | 0% | 70 |

Immunohistochemistry (IHC) stain grade: 0, no; 1+, weak; 2+, moderate; 3+, strong.
CR, complete response; PD, progressive disease.

### Whole Genome Sequencing (WGS) and Analysis of Eleven Relapsed/Refractory Natural Killer/T-Cell Lymphoma (NKTL) Pembrolizumab-Treated Patients

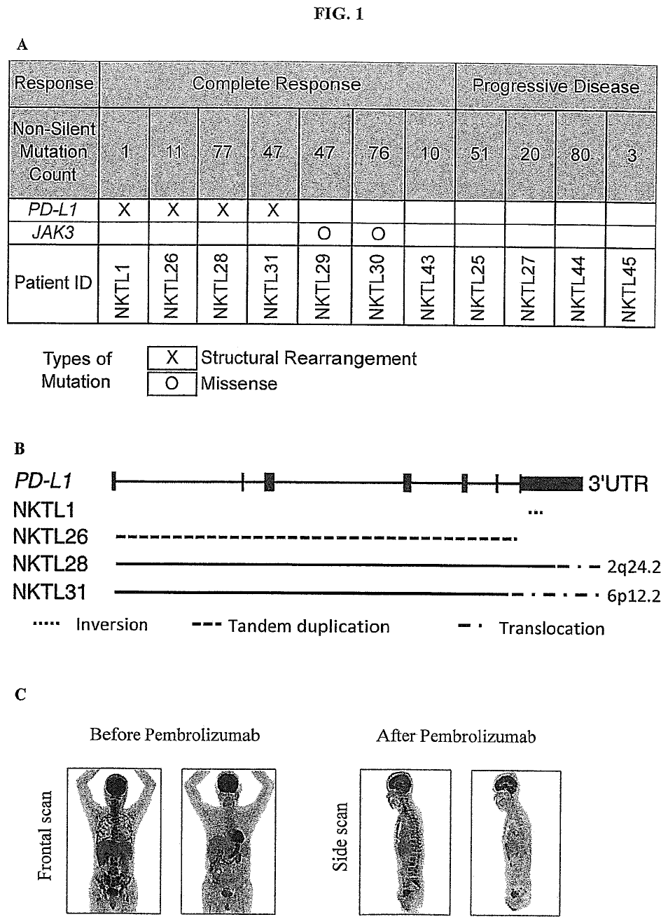

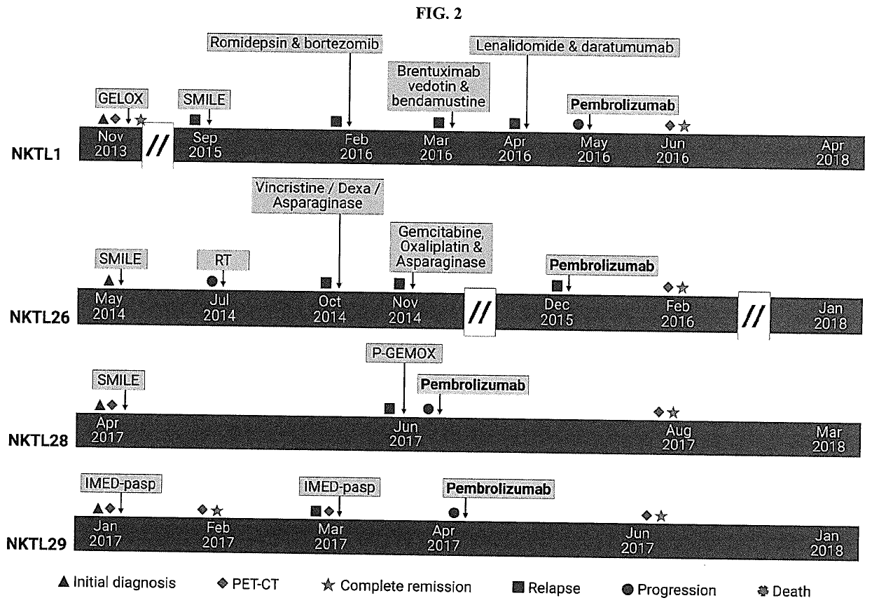

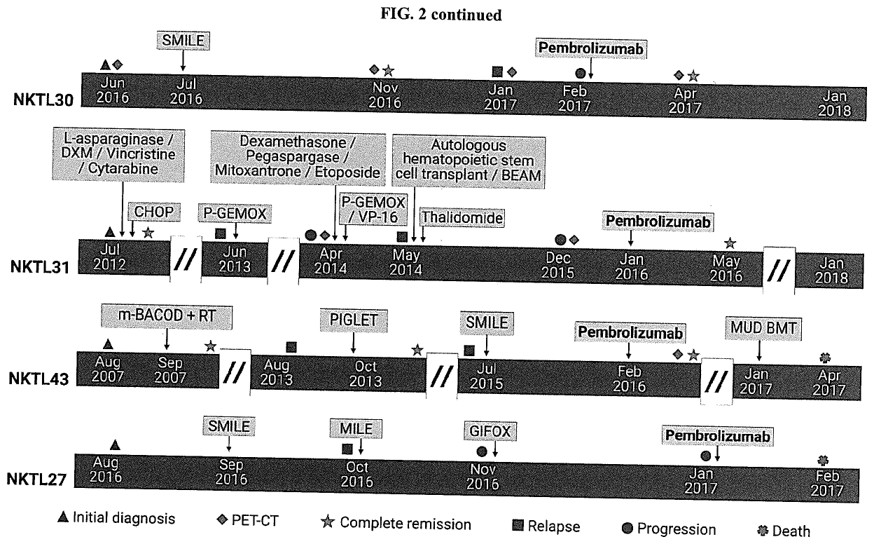

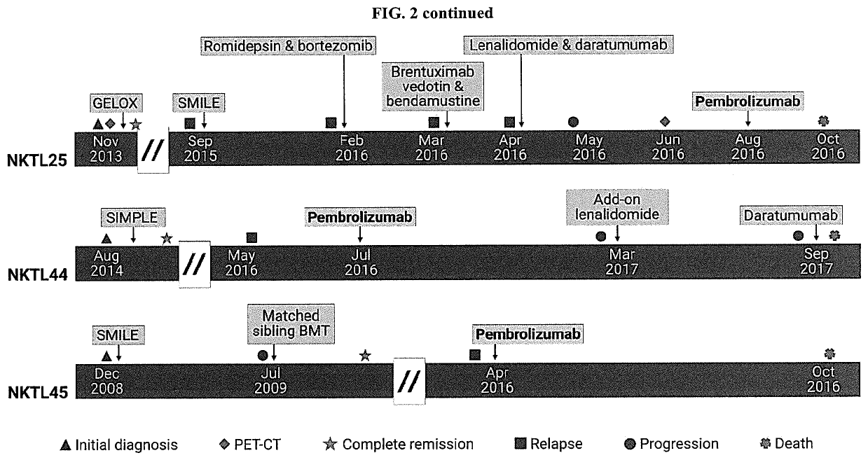

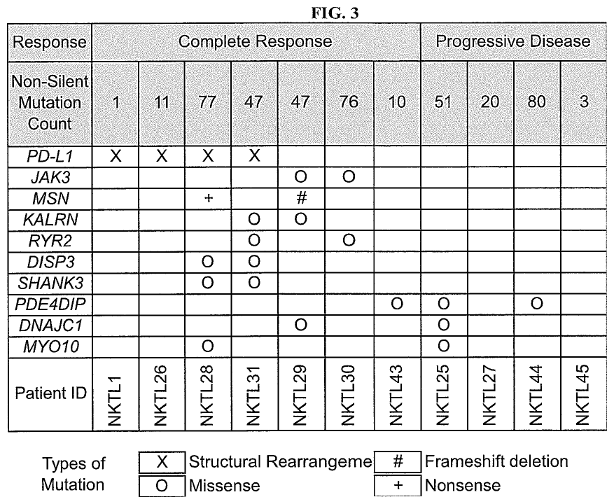

To identify genomic biomarkers of response to PD-1 blockade therapy in natural killer/T-cell lymphoma (NKTL), whole genome sequencing was performed on tumour-normal paired samples obtained from eleven patients who were subsequently treated with pembrolizumab. The natural killer/T-cell lymphoma (NKTL) tumours and, whole blood or buccal swabs, were sequenced to an average depth of 66.6× and 37.5×, respectively (Table 3). Somatic variant calling yielded an average of 1.15 single nucleotide variants (SNVs) and microlndels per Mb for each paired sample. An average of 39 (range: 1 to 80) somatic non-silent protein-coding variants per sample was identified and is comparable to previous reports on whole-exome sequencing of fresh-frozen natural killer/T-cell lymphoma (NKTL) samples (range: 41 to 42). In total, 10 genes were found to be recurrently mutated (FIG. 3). Among them, only PD-L1 structural rearrangement (SR) and JAK3-activating mutations (p.A573V) were recurrent and mutually exclusive to one another among the initial tumours of patients who achieved complete response. Furthermore, PD-L1 structural rearrangement is the most frequent somatic alteration identified in four of seven (57%) initial tumours of patients who achieved complete response to pembrolizumab (FIG. 1A). These PD-L1 structural rearrangements consist of inter-chromosomal translocations (NKTL28 & NKTL31), tandem duplication (NKTL26) and micro-inversion (NKTL1) that disrupted the 3′UTR of PD-L1 (FIG. 1B). The before and after pembrolizumab therapy exemplary positron emission tomograph-computed tomography (PET-CT) scans of the index patient, NKTL1, who have achieved complete response to pembrolizumab confirms the treatment outcome of this patient (FIG. 1C).

TABLE 3
Statistics of the whole-genome sequencing data in this application.

| | | | Number | Number | Effective |
|---|---|---|---|---|---|
| | | Number | of | of | coverage (in |
| | Sample | of | mapped | mapped | percentage |
| | ID | reads | reads | bases | [%]) |
| Tumour | NKTL1 | 1,873, | 1,841, | 258,994, | 86.33 |
| | | 021,019 | 362,493 | 279,300 | |
| | NKTL25 | 1,652, | 1,649, | 245,074, | 81.69 |
| | | 397,588 | 340,219 | 442,068 | |
| | NKTL26 | 1,949, | 1,932, | 132,757, | 44.25 |
| | | 926,626 | 171,625 | 814,328 | |
| | NKTL27 | 2,151, | 2,138, | 130,771, | 43.59 |
| | | 318,866 | 092,437 | 029,478 | |
| | NKTL28 | 2,758, | 2,744, | 289,808, | 96.60 |
| | | 540,565 | 718,768 | 926,126 | |
| | NKTL29 | 2,671, | 2,650, | 292,756, | 97.59 |
| | | 594,980 | 364,189 | 953,530 | |
| | NKTL30 | 2,762, | 2,753, | 283,914, | 94.64 |
| | | 699,520 | 513,699 | 642,242 | |
| | NKTL31 | 2,628, | 2,620, | 198,926, | 66.31 |
| | | 023,988 | 612,101 | 913,247 | |
| | NKTL43 | 2,201, | 2,181, | 123,128, | 41.04 |
| | | 856,071 | 264,472 | 720,538 | |
| | NKTL44 | 2,235, | 2,216, | 114,743, | 38.25 |
| | | 252,498 | 647,890 | 408,866 | |
| | NKTL45 | 2,226, | 2,212, | 129,014, | 43.00 |
| | | 962,038 | 973,594 | 014,229 | |
| Normal | NKTL1 | 1,611, | 1,570, | 194,852, | 64.95 |
| | | 226,752 | 222,321 | 160,506 | |
| | NKTL25 | 1,623, | 1,610, | 238,862, | 79.62 |
| | | 436,934 | 764,801 | 855,285 | |
| | NKTL26 | 712,553, | 709, | 89,482, | 29.83 |
| | | 555 | 714,472 | 905,017 | |
| | NKTL27 | 523,598, | 459,459, | 57,372, | 19.12 |
| | | 128 | 352 | 191,688 | |
| | NKTL28 | 706,818, | 706,319, | 92,224, | 30.74 |
| | | 570 | 632 | 970,337 | |
| | NKTL29 | 701,960, | 701,168, | 92,202, | 30.73 |
| | | 783 | 353 | 504,306 | |
| | NKTL30 | 644,198, | 643,396, | 84,801, | 28.27 |
| | | 391 | 676 | 435,813 | |
| | NKTL31 | 939,968, | 937,273, | 118,112, | 39.37 |
| | | 348 | 257 | 219,219 | |
| | NKTL43 | 730,890, | 729,734, | 93,122, | 31.04 |
| | | 262 | 222 | 677,260 | |
| | NKTL44 | 653,667, | 652,654, | 84,239, | 28.08 |
| | | 897 | 965 | 863,138 | |
| | NKTL45 | 729,363, | 728,227, | 92,454, | 30.82 |
| | | 145 | 150 | 255,181 | |

Therefore, in one example, there is disclosed a method of treating natural killer/T-cell lymphoma in a subject, the method comprising administering to a subject a therapeutically effective amount of pembrolizumab, wherein the subject is characterised by the presence of at least one JAK3-activating somatic mutation. In another example, the at least one JAK3 activating mutation is an activating somatic mutation. In a further example, there is one JAK3 activating mutation present. In yet another example, the JAK3-activating mutation is p.A573V.

Thus, in one example, the mutation referred to herein is a micro-inversion, inversion, translocation, tandem repeat, or a breakpoint (mutation). In another example, the mutation is a translocation, a tandem repeat (or tandem duplication), or a micro-inversion.

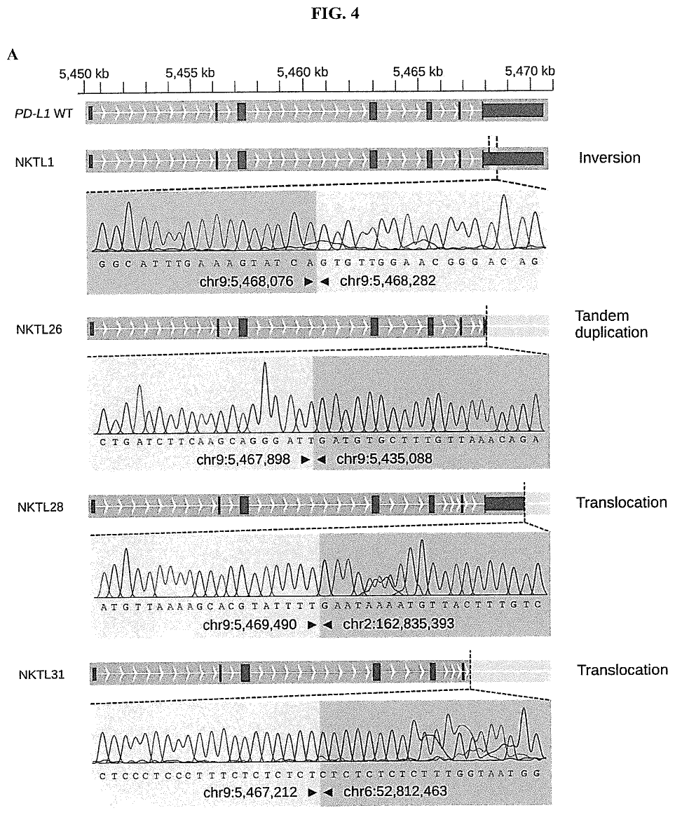

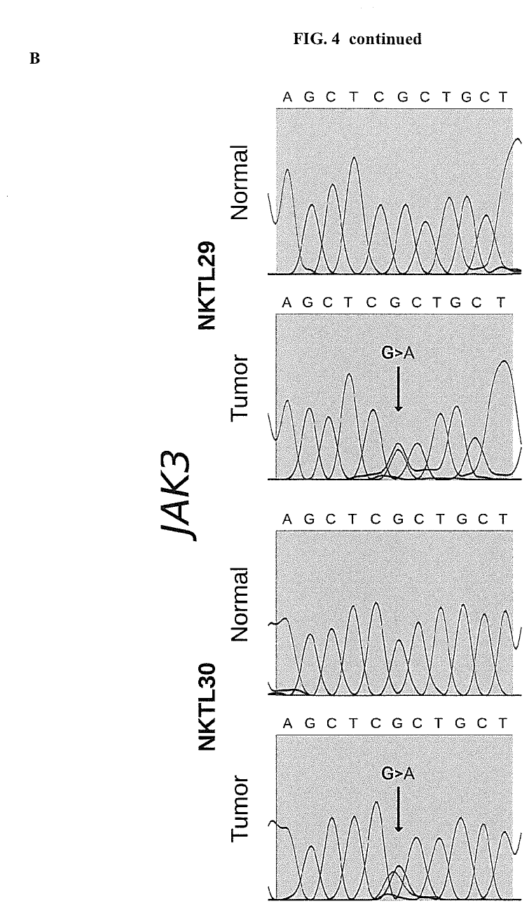

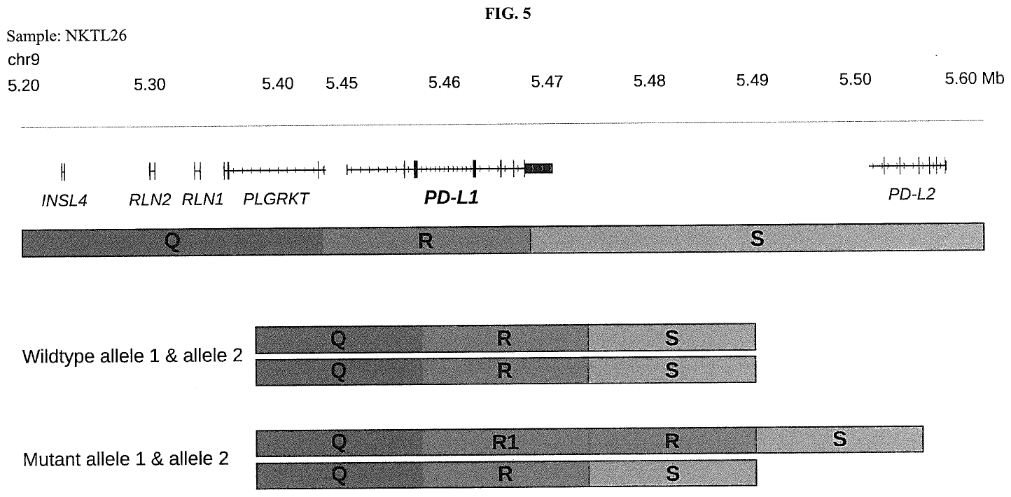

In NKTL28 and NKTL31, exon 7 of PD-L1 was translocated to 2q24.2 and intron 6 of PD-L1 was translocated to 6p12.2, respectively (FIG. 4). In NKTL26, the right breakpoint of tandem duplication was located within the 3′UTR of PD-L1 and the left breakpoint was validated to be about 32 kbp upstream (FIG. 4). This duplication event yielded a copy of 3′UTR-disrupted and wild type copy of PD-L1 (FIG. 5). The final PD-L structural rearrangement in NKTL1 consisted of a 206 bp micro-inversion that sits entirely within the 3′UTR of PD-L1 (FIG. 4). These somatic alterations were absent in the initial tumours from the four patients who had progressive disease with pembrolizumab.

Besides sequence analysis by the inventors' genomic pipeline, visual inspection was also performed for known recurrent mutated genes of natural killer/T-cell lymphoma (NKTL) to avoid artefacts. Mutations in genes associated with antigen presentation and interferon gamma pathways, which are known to associate resistance to immune checkpoint blockade in melanoma, are not found in the analysed cohort.

### Regulatory Activity of PD-L1 3′UTR in Natural Killer/T-Cell Lymphoma (NKTL)

All four PD-L1 structural rearrangements were predicted to lose whole or part of the PD-L1 3′UTR, or the PD-L1 3′UTR function, except the micro-inversion that spanned across 206 bp and sits entirely within the 3′UTR of PD-L1. To determine the functional significance of this micro-inversion in regulating PD-L1 expression, the wild type and mutant (with 206 bp inversion) PD-L1 3′UTR were cloned into a luciferase reporter assay system and transfected into lymphoma and leukemia cell lines, namely, NK-S1, K-562 and Jurkat (FIG. 10A). Results show that the wild type PD-L1 3′UTR can effectively suppress the luciferase activity of the reporter protein and the identified micro-inversion can relieve this suppression in NK-S1, K-562 and Jurkat cell lines (P=0.01, P=0.01 and P=0.03, two-tailed t-test; FIG. 10B). Moderate to high levels (range: 20%-100%) of PD-L1 positivity were observed in these four tumours harbouring PD-L1 3′UTR SR (Table 2). Without being bound by theory, it is thought that these results offer a direct explanation to how these natural killer/T-cell lymphoma (NKTL) tumours evade immune surveillance by up-regulating PD-L1 expression.

### PD-L1 Structural Rearrangements and JAK3-Activating Mutations are Clonal in Natural Killer/T-Cell Lymphoma (NKTL)

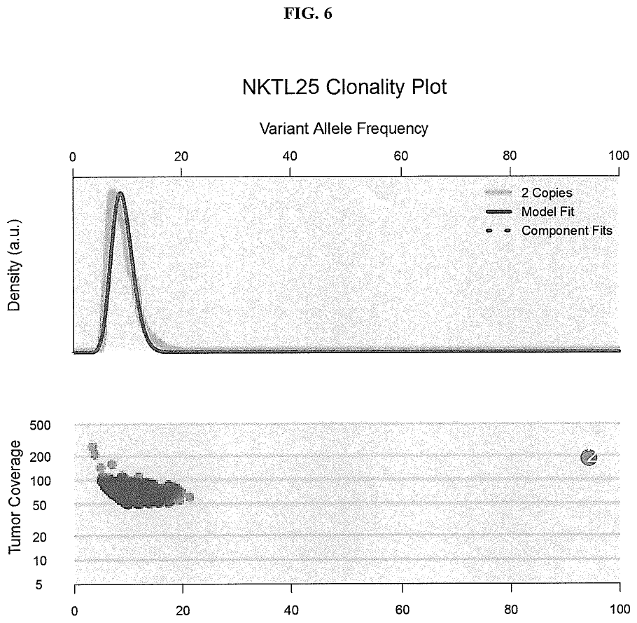

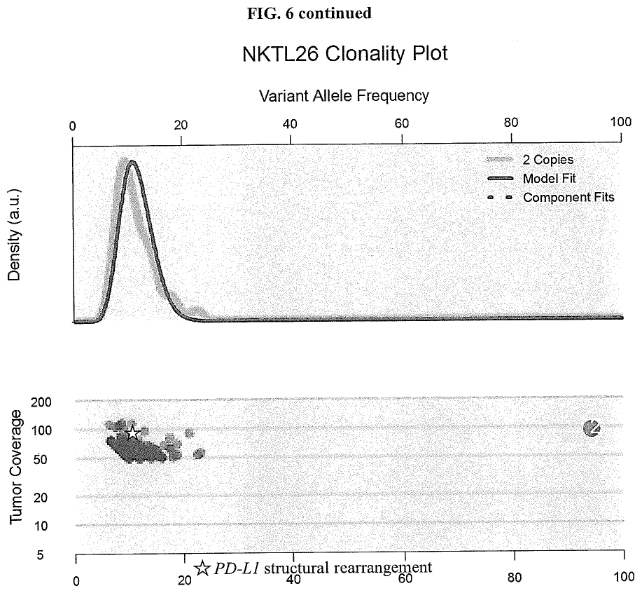

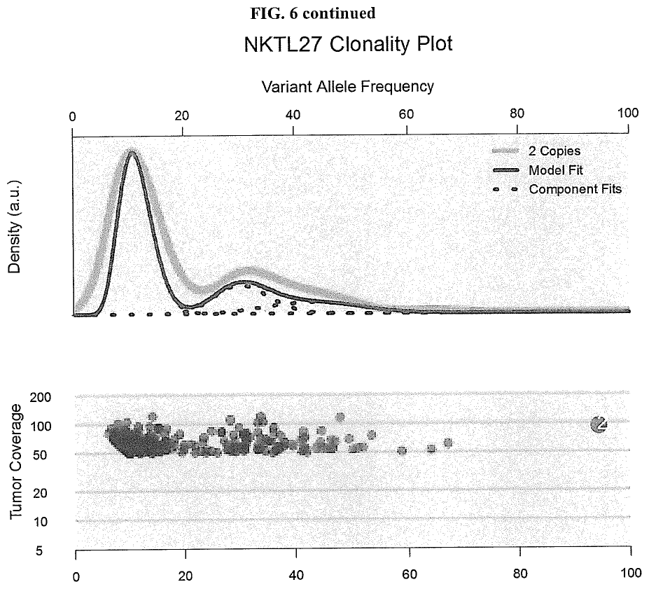

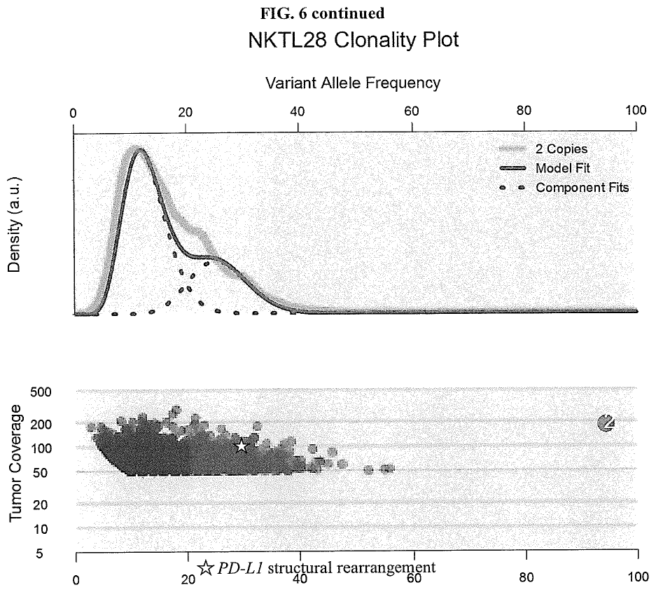

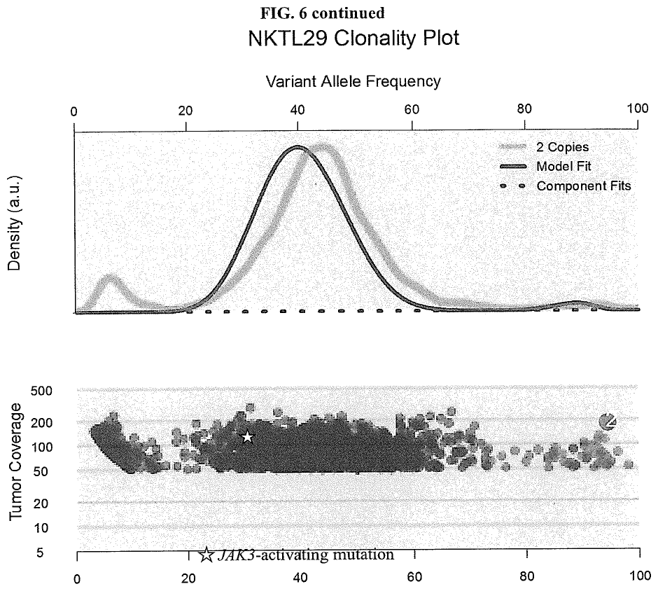

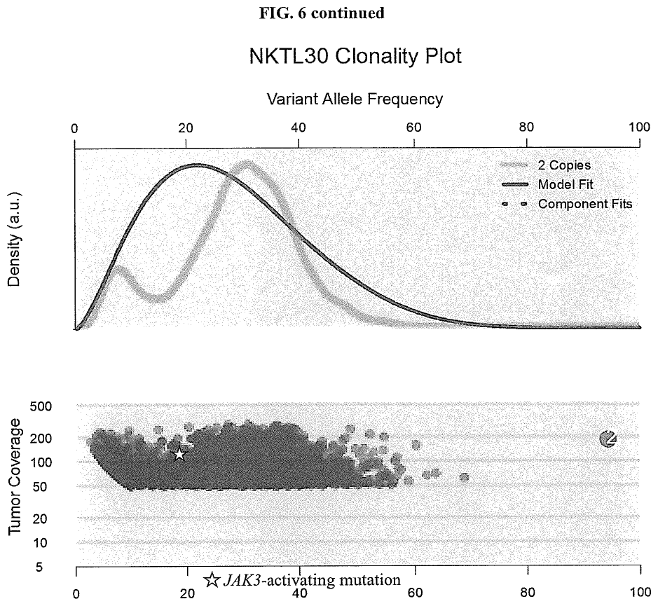

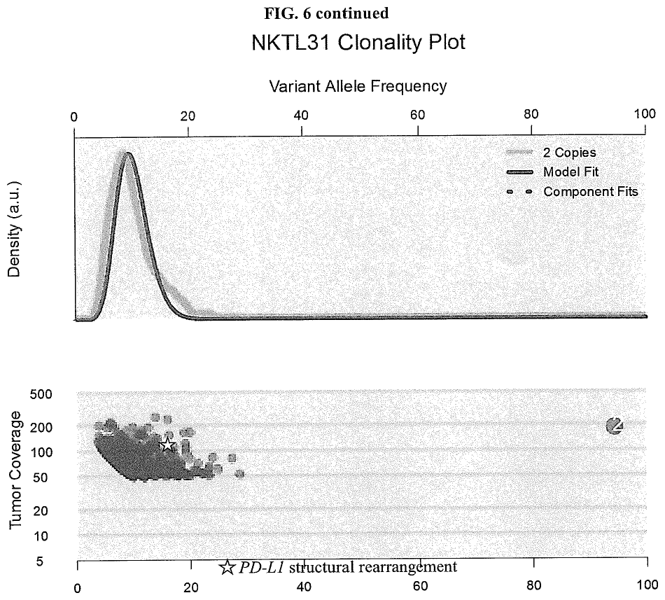

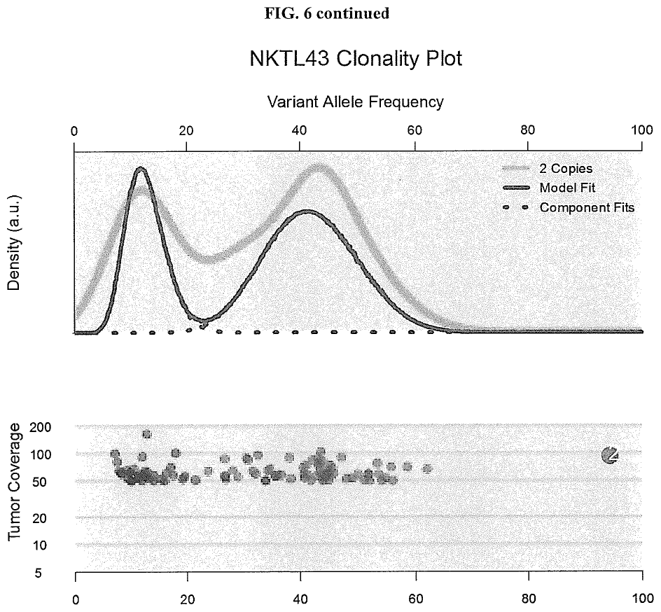

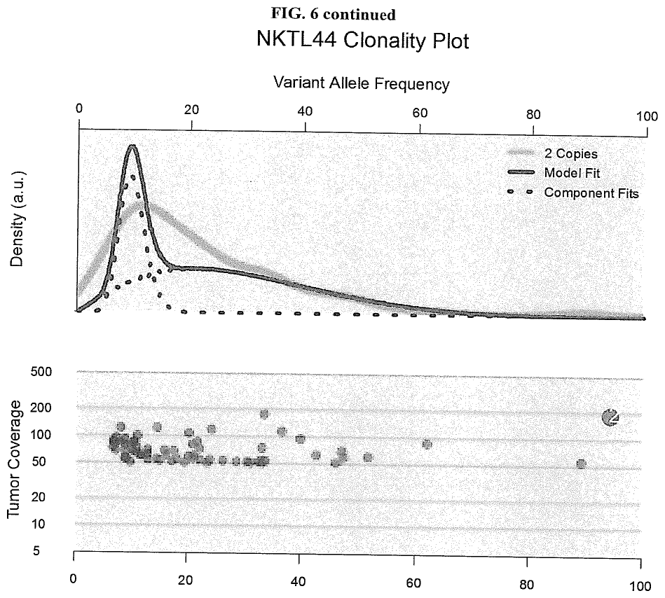

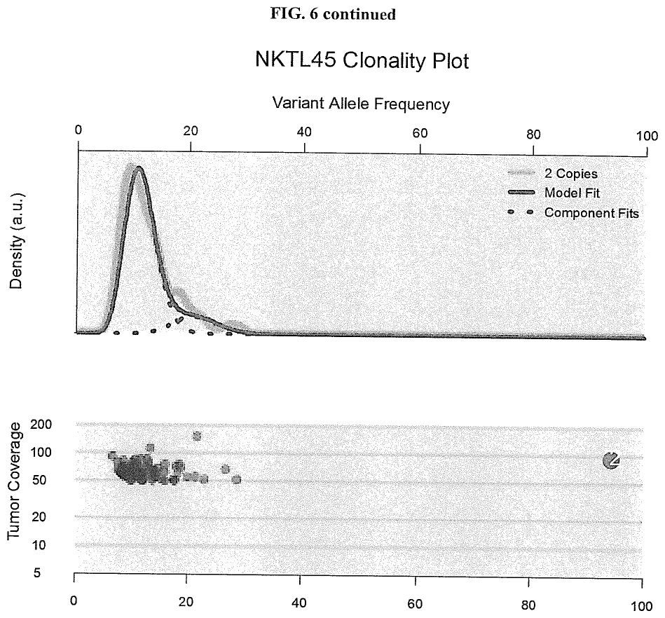

Although the mechanisms of response to PD-1 blockade from PD-L1 3′UTR structural rearrangements and JAK3-activating mutations remain to be elucidated, it was investigated if the clonality of these alterations could support the complete response in patients who had PD-L1 and JAK3 alterations in their pre-treated tumours, from the single-agent regime of pembrolizumab. From the somatic single-nucleotide variants, it was possible to obtain solutions for the clonal architectures for 10 cases (SciClone did not have a clonality solution for NKTL1). Five cases, four complete response cases and one progressive disease cases, had a clonal architecture (Table 4 and FIG. 6). The somatic PD-L1 and JAK3 mutations identified resided in the founding clone of their corresponding pre-treated tumours. Given these results, it is thought that the clonality analysis does support the extent of response in patients who achieved complete response from the single-agent regime of pembrolizumab therapy.

TABLE 4
Clonal residencies of the genomic
correlates of response to pembrolizumab
in the pretreated tumours of this study cohort.

| | | | | JAK3- |
|---|---|---|---|---|
| | | Number | PD-L1 | activating |
| Sample | Response to | of | rearrangement | mutation |
| ID | Pembrolizumab | Clones | clonal? | clonal? |
| NKTL1 | CR | no | Yes | — |
| | | solution | | |
| NKTL26 | CR | 1 | Yes | — |
| NKTL28 | CR | 2 | Yes | — |
| NKTL31 | CR | 1 | Yes | — |
| NKTL29 | CR | 1 | — | Yes |
| NKTL30 | CR | 1 | — | Yes |
| NKTL43 | CR | 2 | — | — |
| NKTL25 | PD | 1 | — | — |
| NKTL27 | PD | 3 | — | — |
| NKTL44 | PD | 2 | — | — |
| NKTL45 | PD | 2 | — | — |

CR, complete response;
PD, progressive disease.

TABLE 5
PD-L1 and PD-L2 alterations described in hematological malignancies.

| Disease | PD-L1 | PD-L2 | Reference |
|---|---|---|---|
| NKTL | Complete loss or disruption | Rearrangements | This |
| | of 3′ UTR | not identified | application |
| | Smaller scale | | |
| | 5′ fusion partner | | |
| | No copy number variations | | |
| ATLL | Complete loss or disruption | Rearrangements | Kataoka et al. |
| | of 3′ UTR | not identified | Nature 2016 |
| | Larger scale | | |
| | 5′ fusion partner | | |
| DLBCL | No copy number variations | | |

| | | |
|---|---|---|
| HL | Chromosomal amplification of 9p24.1 | Green et al. |
| PMBL | (involves, PD-L1, PD-L2 and JAK2) | Blood 2010 |
| DLBCL | Various structural rearrangements | Chong et al. |
| PMBL | Small and large scale | Blood 2016 |
| PTL | 5′ or 3′ fusion partner | |
| PCNSL | Some copy number variations | |

ATLL, adult T-cell leukemia/lymphoma;
DLBCL, diffuse large B-cell lymphoma;
HL, Hodgkin lymphoma;
NKTL, natural killer/T-cell lymphoma;
PCNSL, primary central nervous system lymphoma;
PMBL, primary mediastinal B-cell lymphoma;
PTL, primary testicular lymphoma;
UTR, untranslated region.

Immunotherapy, in particular PD-1 blockade therapy, has shown promise in the treatment of several cancers, including natural killer/T-cell lymphoma. It is shown that four out of seven NKTL patients (57%) who achieved complete response to PD-1 blockade had a clonal architecture for the PD-L1 3′UTR structural rearrangement in their tumours. PD-L1 3′UTR structural rearrangements was also recently identified in a single case of ovarian cancer where the patient achieved complete response with pembrolizumab, further supporting its role as a potential biomarker of response to PD-1 blockade therapy in natural killer/T-cell lymphoma.

Also disclosed herein is a method of determining response of a subject suffering from natural killer/T-cell lymphoma to pembrolizumab treatment, the method comprising obtaining a sample from the subject; detecting the presence or absence of at least one JAK3 activating mutation or at least one PD-L1 structural rearrangement. In another example, the presence of at least one JAK activating mutation or at least one PD-L1 structural rearrangement indicates that the subject will respond to treatment. In another example, the treatment is a compound or treatment as disclosed herein. In yet another example, the treatment is pembrolizumab.

As used herein, the term “response” can also be used interchangeably with susceptibility to a treatment. The term “susceptibility” refers to the propensity of something, for example a disease, to be likely affected by something else, for example, a treatment for said disease. This effect can be either positive or negative, depending on the feature or the treatment which is being referenced. For example, if a subject is sensitive to a particular treatment, then the susceptibility of said subject to a particular treatment is a positive effect. The term “susceptibility” can be interchanged with, for example, reactivity or sensitivity.

Thus, in one example, the method disclosed herein is a method of determining susceptibility of a subject suffering from natural killer/T-cell lymphoma to pembrolizumab treatment.

All natural killer/T-cell lymphomas are diagnostically EBER+(indicating the presence of the Epstein-Barr virus) and the Epstein-Barr virus (EBV) protein, LMPI can be considered to constitutively up-regulate PD-L1. Without being bound by theory, it speculated that natural killer/T-cell lymphomas will respond to PD-1 inhibitors, as they are innately PD-L1+. Indeed, relapsed/refractory natural killer/T-cell lymphoma patients in a previous clinical study had an initial response to pembrolizumab.

Thus, in one example, there is disclosed a method of treating natural killer/T-cell lymphoma in a subject. In another example, the method comprises administering to a subject an inhibitor selected from the group consisting of PD-1 inhibitor, CD279 inhibitor, PD-L1 inhibitor, CD274 inhibitor and combinations thereof. In yet another example, the subject is to be administered an inhibitor selected from the group consisting of PD-1 inhibitor, CD279 inhibitor, PD-L1 inhibitor, CD274 inhibitor and combinations thereof.

Also disclosed herein is the use of a compound or inhibitor as disclosed herein in the manufacture of a medicament for treating natural killer/T-cell lymphoma.

As used herein, the term “inhibitor” refers to compounds that are capable of inhibiting or blocking the activity of a specific receptor, or a group of related receptors. Various compounds and drugs are not limited to a single effect and can therefore be considered to be inhibitors of the same receptor, even if they are structurally and/or chemically different. That is to say, the inhibition of a specific receptor is the characteristic of these compounds in examples where more than one inhibitor is used.

Thus, in one example, the inhibitor as disclosed herein is an inhibitor that results in a blockade of the PD-1/PD-L1 axis. In another example, the inhibitor is, but is not limited to, PD-1 inhibitor, CD279 inhibitor, PD-L1 inhibitor, CD274 inhibitor, and combinations thereof. In yet another example, the method as disclosed herein comprises administering to a subject an inhibitor that is, but is not limited to, PD-1 inhibitor, CD279 inhibitor, PD-L1 inhibitor, CD274 inhibitor and combinations thereof.

As used therein, the term “treatment” refers to both prophylactic inhibition of initial infection or disease, and therapeutic interventions to alter the natural course of an untreated infection or disease process, such as a tumour growth or an infection with a bacteria. Treating a disease also refers to a therapeutic intervention that inhibits, or suppresses, for example, the growth of a tumour, eliminates a tumour, ameliorates at least one sign or symptom of a disease or pathological condition, or interferes with a pathophysiological process, after the disease or pathological condition has begun to develop.

In one example, the treatment or the compound to be administered to the subject is a compound which impedes the PD-1/PD-L1 axis. In other words, these compounds target immune checkpoints that have an effect on subject response to treatment. In one example, these target immune checkpoints are co-inhibitory immune checkpoint molecules. In another example, these co-inhibitory immune checkpoint molecules are, but are not limited to CTLA-4, CD80/CD86, PD1, PD-L1/PD-L2, CD80, PD-L1, BTLA, HVEM, TIM3, and GAL9. In a further example, the treatment or the compound to be administered to the subject is a PD1/PD-L1 blockade therapy. In yet another example, the PD1/PD-L1 blockade therapy is a PD-1 blockade therapy.

Thus, in one example, the treatment or the compound to be administered to the subject is a compound which impedes the PD-1/PD-L1 axis. In another example, the treatment or the compound to be administered to the subject is a compound which targets PD-1. These compounds can be, but are not limited to, nivolumab (opdivo), pembrolizumab (keytruda), atezolizumab (tecentriq), avelumab (bavencio), durvalumab (imfinzi), pidilizumab (Cure Tech), AMP-224 (GlaxoSmithKline), AMP-514 (GlaxoSmithKline), PDR001 (Norvartis), cemiplimab (Regeneron and Sanofi), and combinations thereof. In one example, the compound to be administered is pembrolizumab (keytruda) in combination with any other compounds as disclosed herein. In another example, the compound is pembrolizumab (keytruda).

Subsequently, four of the eleven patients have progressed and died of disease. Alterations of the PD-L1 and JAK3 genes in these progressive cases had not been found. Without being bound by theory, it is thought that this initial “pseudo-remission” could be attributed by exogenous factors, such as Epstein-Barr virus (EBV) up-regulating PD-L1 that was transiently blocked by initial dosages of pembrolizumab. Hence, high PD-L1 positivity in tumours will not necessarily equate to good response to PD-1 blockade. In addition, the PD-L1 immunohistochemistry scores also varied greatly (6%, 2+ to 100%, 3+) within the cohort, and both subjects NKTL25 and NKTL27 had progressive disease despite having high PD-L1 staining grade for their pre-treated tumours, resulting in questions being raised to the effectiveness of PD-L1 positivity alone as a biomarker of response to PD-1 blockade in natural killer/T-cell lymphoma. No rearrangements were identified within the PD-L2 gene, and PD-L1 always served as the 5′ rearrangement partner with regard to structural rearrangements. This is in contrast to other hematologic malignancies where the over-expression of PD-L1 and/or PD-L2 is achieved by diverse mechanisms such as genomic amplification, JAK2 or PD-L2 translocations (Table 5), suggesting that different tumours have evolved alternate mechanisms for immune evasion.

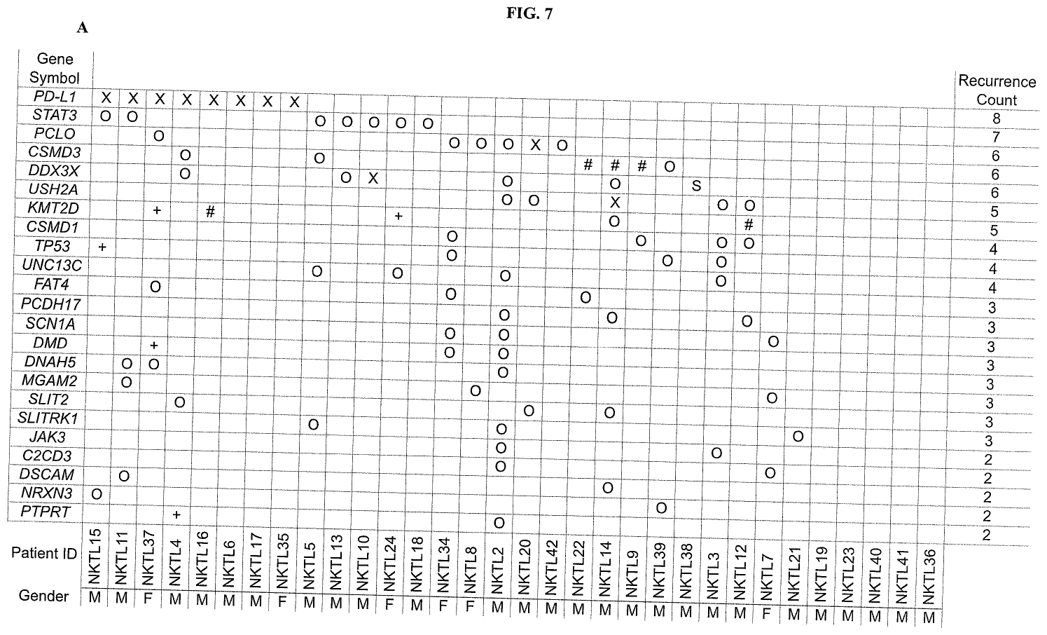

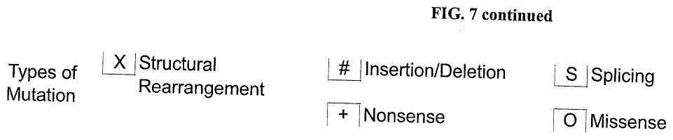

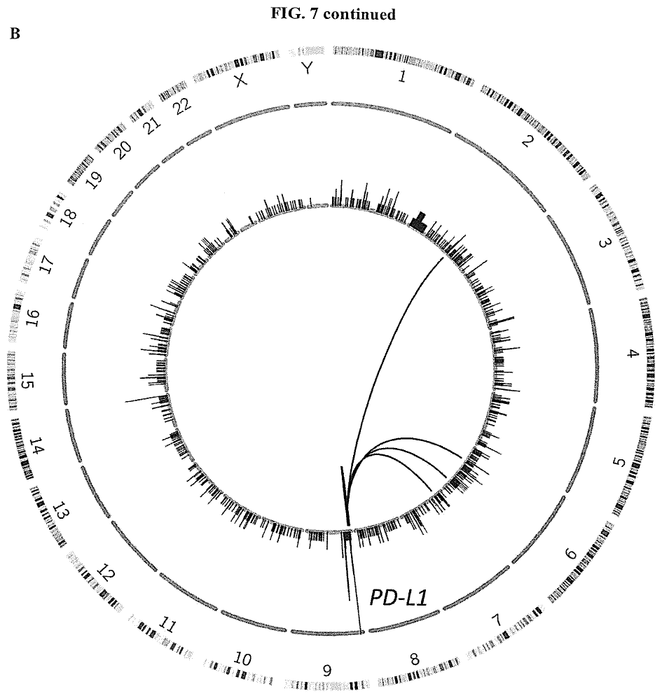

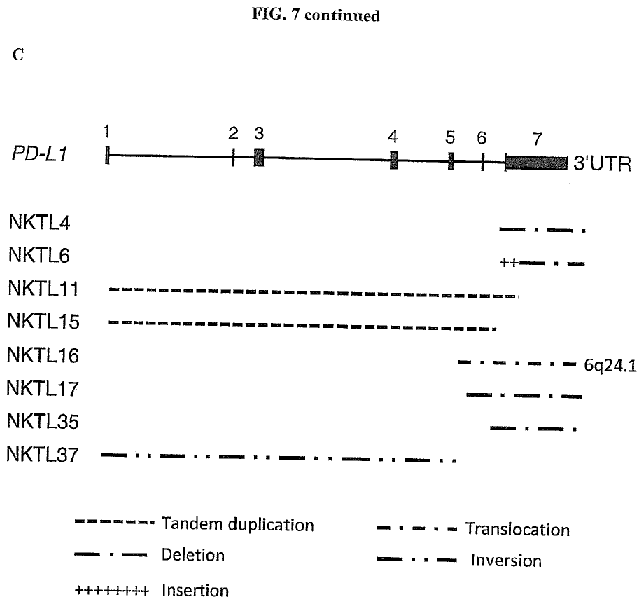

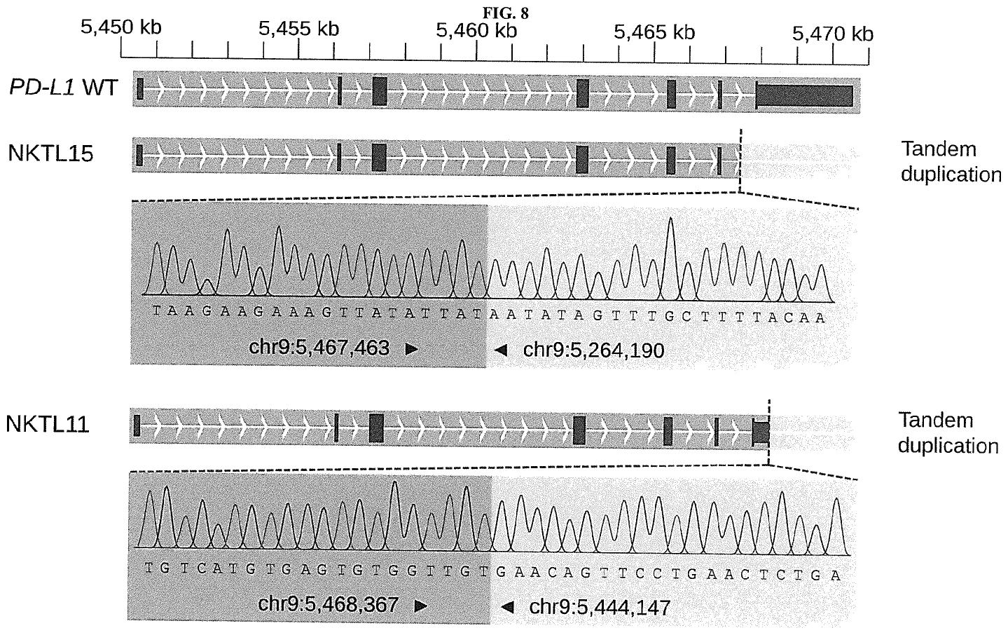

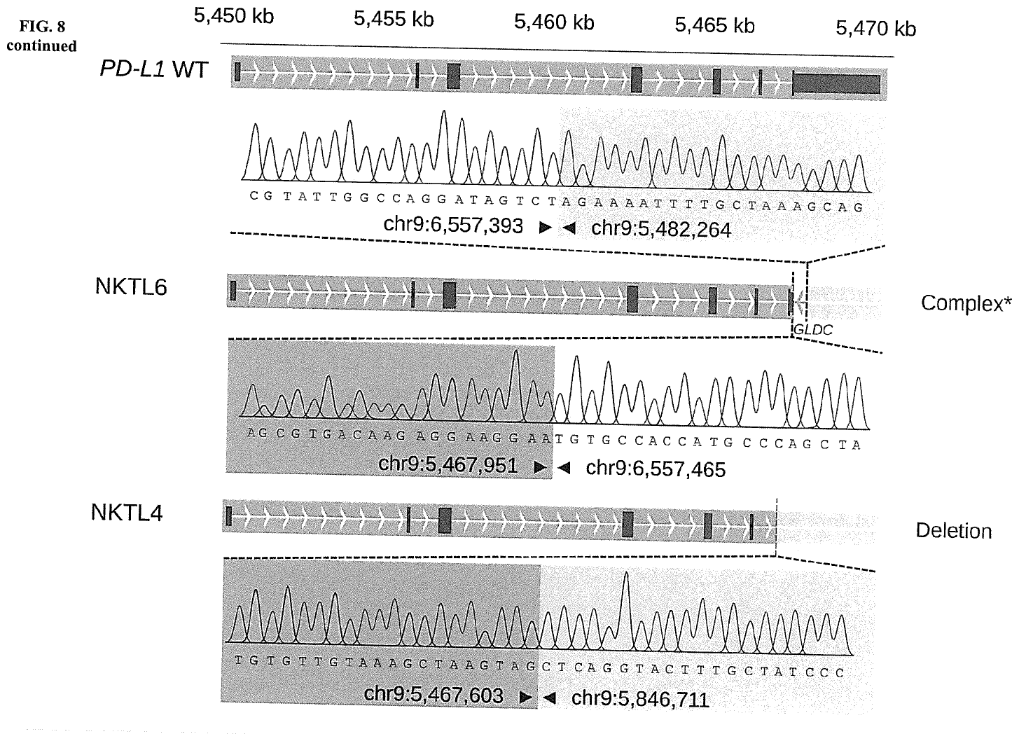

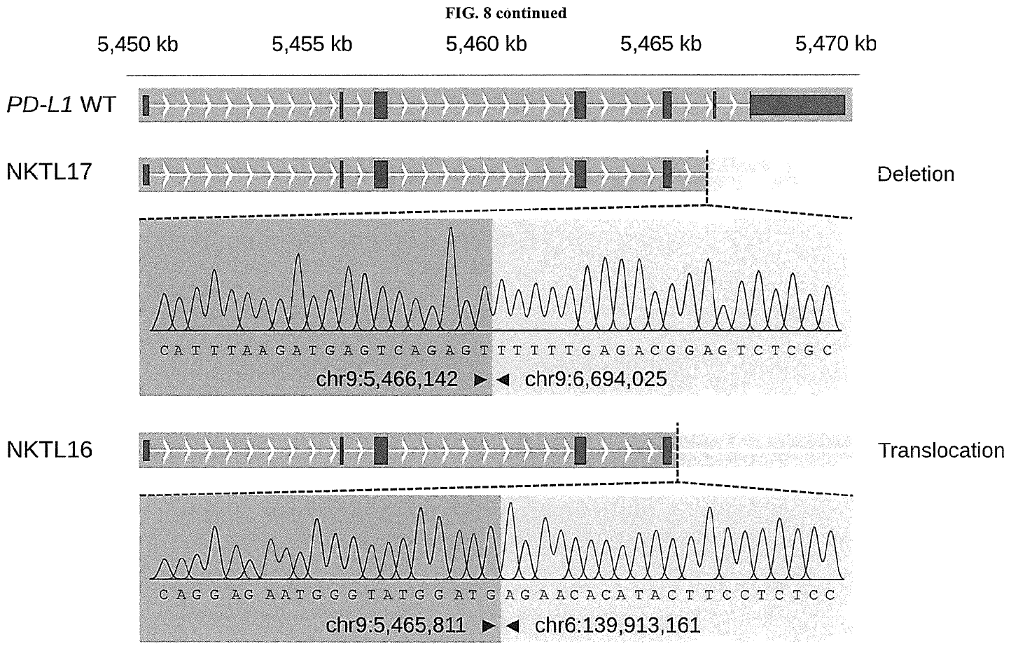

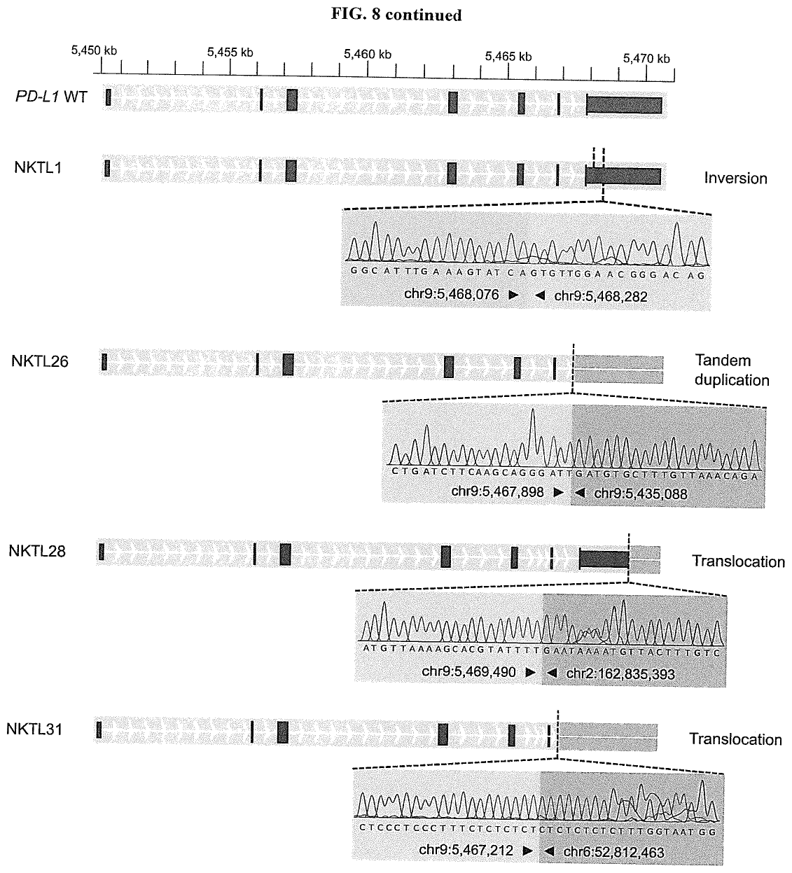

To determine the prevalence of PD-L1 and JAK3 alterations, whole-genome sequencing (WGS) was performed on 32 more paired tumour-normal natural killer/T-cell lymphoma (NKTL) tumours and corresponding peripheral blood lymphocytes, the clinicopathological information of which is listed in Table 6 below. The absence of malignant cells in the corresponding peripheral blood in these samples was verified by mapping the sequencing data to the EBV genome as the pathogenic virus is known to reside in the neoplastic cells. Similar to the cohort of 11 pembrolizumab-treated patients, in this extended cohort of 32 NKTL samples that had no subsequent pembrolizumab treatment; PD-L1 was also found to be the most recurrently altered gene in the cohort (FIG. 7A). In terms of structural rearrangement, PD-L1 also stood out significantly as being the most rearranged gene (FIG. 7B). The form of alterations to PD-L1 in these natural killer/T-cell lymphomas (NKTL tumours) involves a structural breakpoint cluster within the genomic region of PD-L1 that was present in 25% (8 of 32) of the cases (FIG. 7C). All of the structural rearrangements that were found within the locus of PD-L1 were validated using Sanger sequencing (FIG. 8). The bioinformatics analysis has also re-identified recurrent non-silent short variants in genes, such as TP53, DDX3X, STAT3, FAT4 and JAK3 (6.3%, 2 of 32), suggesting similar pathology with previous studied cohorts.

TABLE 6
Clinicopathological information of patients

Gender

| | |
|---|---|
| Total number with data | 40 |
| Female | 6 (15%) |
| Male | 34 (85%) |

Age (years)

| | |
|---|---|
| Total number with data | 40 |
| Median (range) | 42 (18-82) |

Stage

| | |
|---|---|
| Total number with data | 40 |
| I and II | 26 (65%) |
| III and IV | 14 (35%) |

Elevated LDH

| | |
|---|---|
| Total number with data | 30 |
| No | 15 (50%) |
| Yes | 15 (50%) |

International Prognostic Index

| | |
|---|---|
| Total number with data | 28 |
| Low and low-intermediate risk | 22 (79%) |
| High and high-intermediate risk | 6 (21%) |

ECOG Performance Status

| | |
|---|---|
| Total number with data | 28 |
| 0-2 | 26 (93%) |
| 3-4 | 2 (7%) |

Treatment

| | |
|---|---|
| Total number with data | 37 |
| Chemotherapy | 19 (51%) |
| RT | 1 (2.7%) |
| Chemotherapy and RT | 12 (32%) |
| Chemotherapy, RT and allogeneic SCT | 2 (5.4%) |
| High-dose chemotherapy and autologous SCT | 1 (2.7%) |
| High-dose chemotherapy, autologous SCT and RT | 1 (2.7%) |
| High-dose chemotherapy, autologous SCT, | 1 (2.7%) |
| RT and allogeneic SCT | |

Overall Survival (months)

| | |
|---|---|
| Total number with data | 34 |
| Median (95% CI) | 22.9 (14.4-UD) |

Progression-Free Survival (months)

| | |
|---|---|
| Total number with data | 35 |
| Median (95% CI) | 26.93 (7.82-UD) |

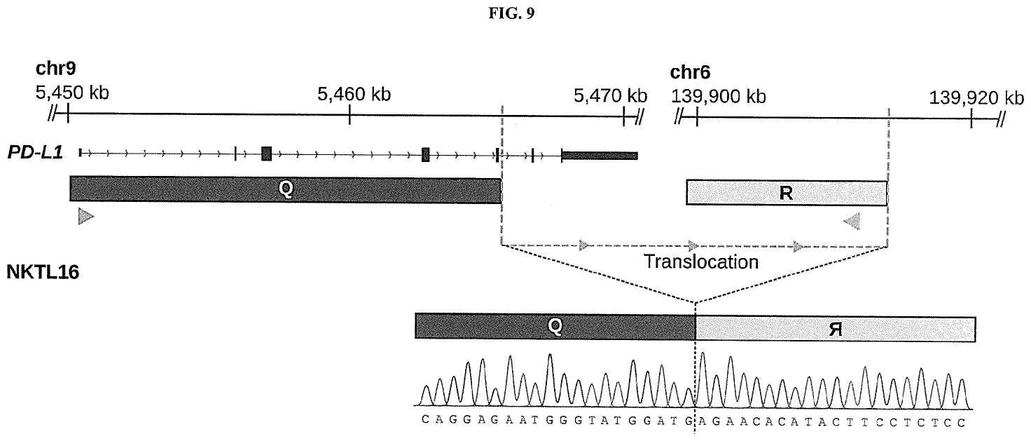

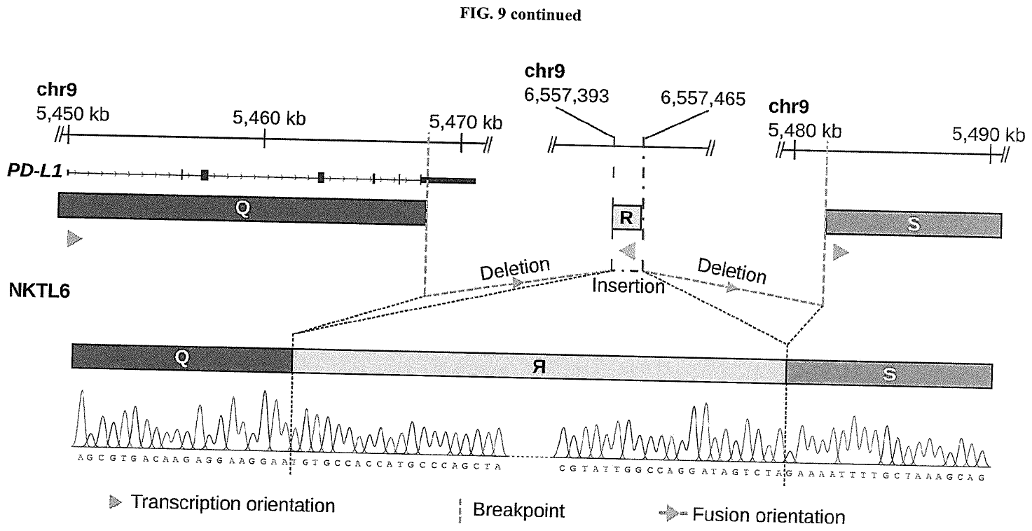

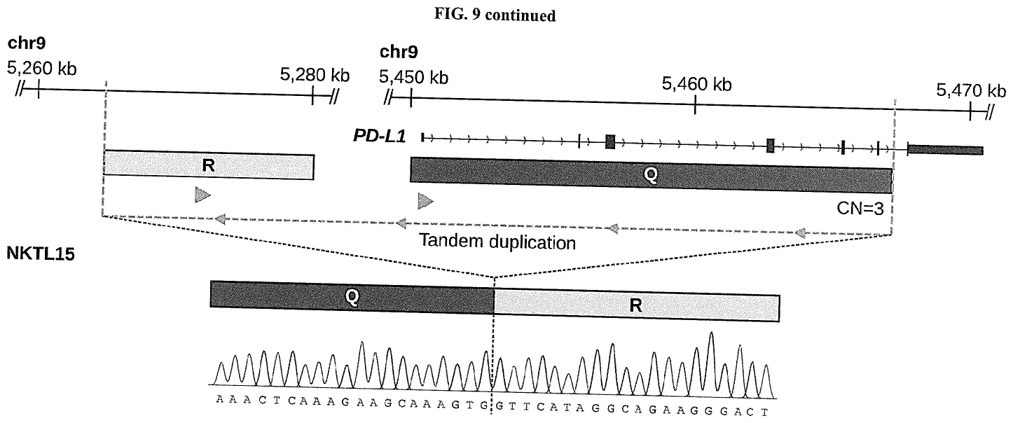

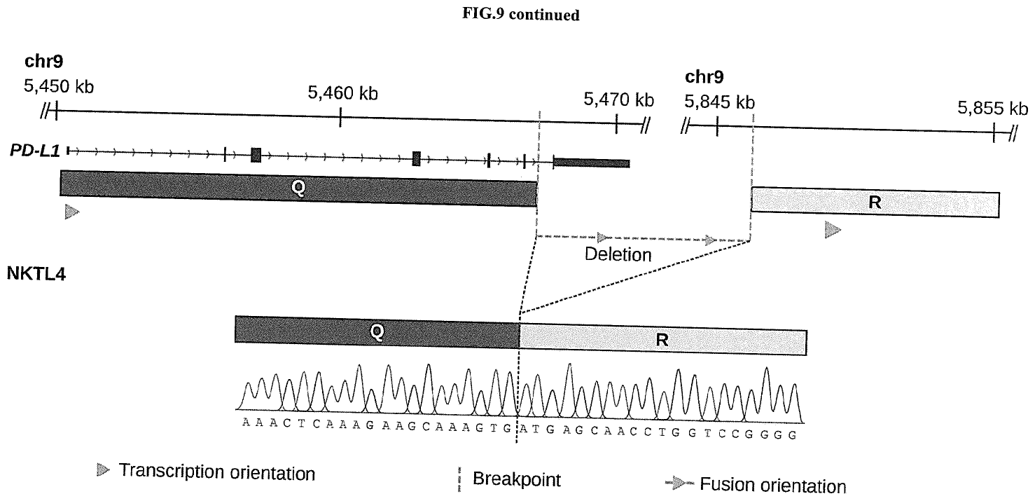

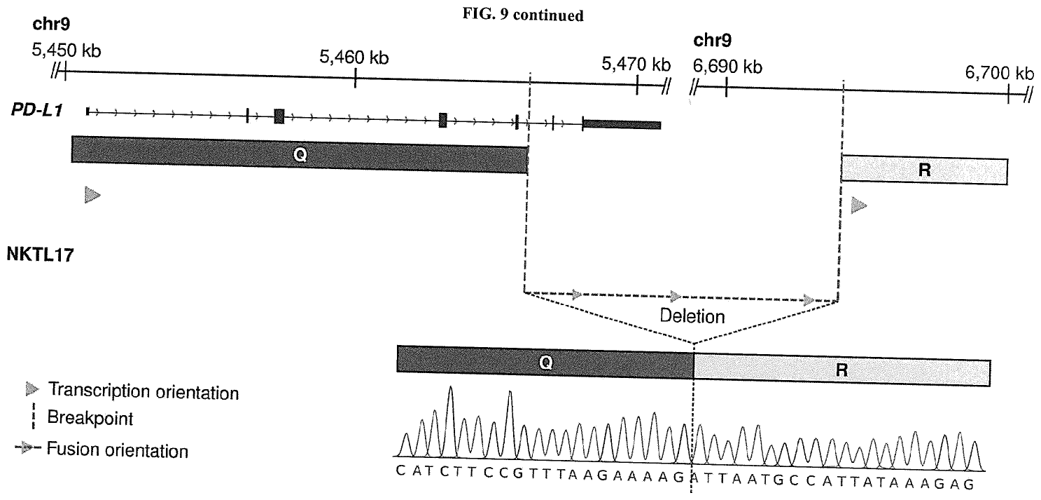

The presence of aberrant transcripts in tumours harbouring PD-L1 3′UTR structural rearrangement (SR) was determined. For each of the PD-L1 SR, with available whole transcriptomic sequencing (WTS) data, it was possible to identify and validate the PD-L1 chimeric transcripts by Sanger sequencing (FIG. 9).

Also disclosed herein is a kit for performing the method described herein. Thus, in one example, there is disclosed a kit for detecting the presence or absence of at least one JAK3 activating mutation or at least one PD-L1 structural rearrangement, the kit comprising a detection agent, and at least one pair of primers. In yet another example, there is disclosed a kit or detecting the presence or absence of at least one JAK3 activating mutation or at least one PD-L1 structural rearrangement comprising a detection agent, and at least one pair of primers; wherein the primers enrich for the genomic regions of the JAK3 and PD-L1 genes.

In one example, the at least one pair of primers is, but is not limited to, the primer pairs as listed in Tables 8 and 9 of the present specification. In another example, the primer pairs are, but are not limited to, SEQ ID NO: 1 and 2, SEQ ID NO: 3 and 4, SEQ ID NO: 5 and 6, SEQ ID NO: 7 and 8, SEQ ID NO: 9 and 10, SEQ ID NO:11 and 12, SEQ ID NO:13 and 14, SEQ ID NO:15 and 16, SEQ ID NO:17 and 18, SEQ ID NO: 19 and 20, SEQ ID NO: 21 and 22, SEQ ID NO: 23 and 24, SEQ ID NO: 25 and 26, SEQ ID NO:27 and 28, SEQ ID NO:29 and 30, SEQ ID NO:31 and 32, SEQ ID NO:33 and 34, SEQ ID NO: 35 and 36, SEQ ID NO: 37 and 38, SEQ ID NO: 39 and 40, SEQ ID NO: 41 and 42, SEQ ID NO: 43 and 44, SEQ ID NO: 45 and 46, and SEQ ID NO: 47 and 48. In yet another example, there is disclosed a kit for detecting the presence or absence of at least one JAK3 activating mutation or at least one PD-L1 structural rearrangement for next-generation sequencing. In yet another example, the kit as disclosed herein is for use according to the method as disclosed herein.

In summary, in the full cohort of 43 natural killer/T-cell lymphoma (NKTL) samples (11 samples were subsequently treated with pembrolizumab and 32 samples were not), it is shown that frequent (27.9%, 12 of 43) somatic PD-L1 3′UTR structural rearrangement in extranodal natural killer/T-cell lymphomas can explain how some extranodal natural killer/T-cell lymphomas can evade immune surveillance, thereby providing the foundation to use PD-1 inhibitors to better treat these patients.

The presence of recurrent JAK3-activating mutations in the described complete response cases also coincide with a report showing the long-term benefit of PD-1 blockade in a single lung cancer patient with JAK3-activating mutations.

It is shown that genomic features correlate with response to PD-1 blockade therapy in natural killer/T-cell lymphoma using whole genome sequencing data and showed that patients can be better selected for PD-1 blockade therapy via genomic screening.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used in this application, the singular form “a,” “an,” and “the” include plural references unless the context clearly dictates otherwise. For example, the term “a genetic marker” includes a plurality of genetic markers, including mixtures and combinations thereof.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

The following examples illustrate methods by which aspects of the invention may be practiced or materials that may be prepared which is suitable for the practice of certain embodiments of the invention.

### Example 1—Materials and Methods

### Patients and Methods

Eleven relapsed or refractory (RR) natural-killer/T-cell lymphoma patients were treated with pembrolizumab. Responses were assessed by radiological scans with the RECIST criteria. Whole genome sequencing (WGS) was used to molecularly profile all the pre-pembrolizumab tumours and matching normals of the eleven patients.

### Study Design

For relapsed or refractory (RR) natural-killer/T-cell lymphoma, the study cohort consists of 11 patients with relapsed or refractory (RR) natural-killer/T-cell lymphoma who had failed L-asparaginase-based chemotherapy regimens from Singapore, China and Hong Kong. NKTL1, NKTL25, NKTL26, NKTL43, NKTL44 and NKTL45 which were not previously sequenced were included from the previous study. Patients were diagnosed with natural-killer/T-cell lymphoma according to the 2008 World Health Organization classification with cytotoxic, CD3ε+ and EBER+ phenotypes. Initial tumours and blood/buccal swabs samples of 43 extranodal natural killer/T-cell lymphoma patients were collected, of which, 11 of them who have failed L-asparaginase-based chemotherapy regimens were subsequently treated with pembrolizumab. Response assessment was performed using a combination of PET/CT or CT/MRI or EBV PCR. Whole genome sequencing was used to molecularly profile all the pre-pembrolizumab tumours and matching normal pairs. The duration of response (DoR) was calculated from the date of starting pembrolizumab to the date of progression or death. The median DoR was estimated using the Kaplan-Meier method. Institutional Review Boards from SingHealth (2004/407/F), National University of Singapore (NUS-IRB-10-250) and Sun Yat-sen University Cancer Center (YB2015-015-01) approved the study. All subjects in this study provided written informed consent. The study also adhered to the Declaration of Helsinki.

For extranodal natural killer/T-cell lymphoma, all subjects in the study provided written informed consent. Extranodal natural killer/T-cell lymphoma was diagnosed according to the 2008 World Health Organization classification with cytotoxic, CD3ε+ and EBER+ phenotypes 3. Institutional Review Boards from SingHealth (2004/407/F), National University of Singapore (NUS-IRB-10-250) and Sun Yat-sen University Cancer Center (YB2015-015-01) approved the study. Initial tumours and blood samples of 40 extranodal natural killer/T-cell lymphoma patients were collected, of which, six of them were also treated with pembrolizumab after they have progressed onto the relapsed or refractory (RR) status. Four of these pembrolizumab-treated patients were from Singapore and the remaining two patients were from China. A combination of physical signs (for example, peripheral blood EBV loads and, PET or CT scans) was used to determine clinical response for pembrolizumab-treated patients. Among these six patients, fresh-frozen tumours were available for one patient and formalin-fixed paraffin-embedded (FFPE) tissues were available for five patients. WGS data was generated for all 40 tumours-blood samples. Sequencing and alignment statistics can be found in Table 7.

TABLE 7
Sequencing and alignment statistics

| | | | | | | | % of |
|---|---|---|---|---|---|---|---|
| | | | | | | | reference |
| | | | | | | | genome |
| | | Number of | Number of | Number of | Number of | Mean | covered >= |
| | Sample ID | reads | mapped reads | mapped bases | duplicated reads | coverage Data | 20X reads |
| Tumours | NKTL1 | 1,873,021,019 | 1,841,362,493 | 258,994,279,300 | 1,627,942,267 | 3.58X | 0.52% |
| | NKTL10 | 1,839,678,190 | 1,834,383,151 | 274,389,980,564 | 630,411,476 | 87.4562X | 92.08% |
| | NKTL11 | 1,578,764,854 | 1,569,189,507 | 233,816,631,859 | 504,381,075 | 74.5243X | 92.07% |
| | NKTL12 | 1,677,294,527 | 1,666,465,566 | 248,419,632,646 | 544,684,376 | 79.1787X | 91.98% |
| | NKTL13 | 1,567,597,744 | 1,538,892,305 | 222,838,551,647 | 98,021,463 | 71.0253X | 91.93% |
| | NKTL14 | 1,630,772,366 | 1,621,410,856 | 241,930,081,229 | 543,979,283 | 77.1103X | 92.03% |
| | NKTL15 | 1,996,443,554 | 1,993,215,581 | 295,250,341,805 | 122,298,728 | 94.1051X | 92.12% |
| | NKTL16 | 1,581,116,784 | 1,578,459,641 | 234,178,914,588 | 79,361,368 | 74.6398X | 91.94% |
| | NKTL17 | 1,338,710,098 | 1,329,376,087 | 195,877,580,658 | 93,231,985 | 62.432X | 91.87% |
| | NKTL18 | 2,057,289,071 | 2,046,896,402 | 301,641,313,462 | 252,005,277 | 96.1421X | 92.15% |
| | NKTL19 | 1,569,852,392 | 1,566,148,685 | 232,327,953,942 | 94,939,566 | 74.0498X | 91.96% |
| | NKTL2 | 1,579,768,935 | 1,568,293,841 | 229,770,266,729 | 89,349,338 | 73.2346X | 91.92% |
| | NKTL20 | 1,535,688,194 | 1,523,364,760 | 224,206,381,893 | 563,399,700 | 71.4612X | 91.96% |
| | NKTL21 | 1,671,250,907 | 1,667,831,285 | 249,215,337,841 | 552,294,432 | 79.4323X | 92.04% |
| | NKTL22 | 1,684,034,777 | 1,680,233,980 | 251,097,847,397 | 558,512,099 | 80.0323X | 92.02% |
| | NKTL23 | 1,665,063,669 | 1,661,934,506 | 248,267,759,786 | 550,234,095 | 79.1303X | 92.02% |
| | NKTL24 | 1,781,409,194 | 1,777,292,380 | 265,727,353,276 | 614,379,035 | 84.6952X | 91.37% |
| | NKTL25 | 1,652,397,588 | 1,649,340,219 | 245,074,442,068 | 282,774,905 | 78.1125X | 92.05% |
| | NKTL26 | 1,949,926,626 | 1,932,171,625 | 132,757,814,328 | 698,325,627 | 42.3139X | 69.72% |
| | NKTL27 | 2,151,318,866 | 2,138,092,437 | 130,771,029,478 | 923,568,143 | 41.6806X | 54.80% |
| | NKTL28 | 2,758,540,565 | 2,744,718,768 | 289,808,926,126 | 487,724,907 | 92.3707X | 82.67% |
| | NKTL29 | 2,671,594,980 | 2,650,364,189 | 292,756,953,530 | 445,960,193 | 93.3103X | 84.56% |
| | NKTL3 | 1,565,216,155 | 1,561,446,509 | 156,206,270,623 | 25,094,854 | 49.7876X | 90.46% |
| | NKTL30 | 2,762,699,520 | 2,753,513,699 | 283,914,642,242 | 586,951,478 | 90.492X | 81.61% |
| | NKTL31 | 2,628,023,988 | 2,620,612,101 | 198,926,913,247 | 889,935,997 | 63.4039X | 58.19% |
| | NKTL4 | 1,513,547,864 | 1,508,342,987 | 150,750,779,458 | 34,134,098 | 48.0488X | 90.51% |
| | NKTL5 | 2,118,619,780 | 2,112,745,129 | 315,179,364,103 | 784,701,131 | 100.457X | 92.04% |
| | NKTL6 | 1,721,320,825 | 1,717,318,733 | 256,289,513,353 | 586,227,378 | 81.6871X | 92.11% |
| | NKTL7 | 1,780,520,536 | 1,771,122,071 | 263,467,557,474 | 285,060,497 | 83.9749X | 91.29% |
| | NKTL8 | 1,346,166,683 | 1,335,796,318 | 195,694,660,644 | 61,277,346 | 62.3737X | 91.16% |
| | NKTL9 | 1,899,248,393 | 1,893,482,953 | 282,701,810,246 | 306,618,154 | 90.1055X | 91.94% |
| | NKTL34 | 1,327,568,329 | 1,324,794,876 | 147,066,020,862 | 328,496,361 | 46.8743X | 90.66% |
| | NKTL35 | 1,428,368,487 | 1,423,731,061 | 152,950,774,137 | 384,080,558 | 48.75X | 91.16% |
| | NKTL36 | 1,511,231,246 | 1,504,969,584 | 162,864,484,788 | 400,373,924 | 51.9098X | 91.46% |
| | NKTL37 | 1,492,150,868 | 1,483,627,647 | 150,813,755,976 | 446,874,296 | 48.0688X | 90.91% |
| | NKTL38 | 1,390,360,799 | 1,385,696,561 | 169,481,294,869 | 238,211,987 | 54.0187X | 91.76% |
| | NKTL39 | 1,456,872,235 | 1,452,717,189 | 173,937,704,395 | 276,280,783 | 55.4391X | 90.90% |
| | NKTL40 | 1,433,780,838 | 1,429,934,638 | 168,652,639,945 | 286,880,239 | 53.7546X | 91.67% |
| | NKTL41 | 1,363,148,313 | 1,360,298,704 | 177,536,662,050 | 157,512,241 | 56.5862X | 91.71% |
| | NKTL42 | 1,508,216,636 | 1,505,956,863 | 187,792,287,863 | 236,185,534 | 59.855X | 91.88% |
| Normal | NKTL1 | 1,611,226,752 | 1,570,222,321 | 194,852,160,506 | 256,766,024 | 62.1052X | 91.94% |
| | NKTL10 | 772,542,143 | 769,549,251 | 114,614,527,683 | 173,694,557 | 36.5311X | 88.35% |
| | NKTL11 | 750,354,707 | 747,031,210 | 111,334,620,961 | 171,847,495 | 35.4857X | 88.01% |
| | NKTL12 | 827,532,205 | 824,701,126 | 122,924,946,417 | 199,718,499 | 39.1798X | 89.11% |
| | NKTL13 | 1,588,660,908 | 1,550,947,735 | 223,967,068,029 | 120,596,122 | 71.385X | 91.63% |
| | NKTL14 | 932,575,929 | 929,709,626 | 138,545,148,179 | 280,447,466 | 44.1585X | 90.14% |
| | NKTL15 | 812,931,406 | 811,402,766 | 120,016,995,417 | 46,590,319 | 38.253X | 88.76% |
| | NKTL16 | 816,399,587 | 815,258,758 | 120,595,575,773 | 48,499,769 | 38.4374X | 88.69% |
| | NKTL17 | 842,535,354 | 836,568,311 | 123,295,177,938 | 67,793,561 | 39.2978X | 88.96% |
| | NKTL18 | 798,917,784 | 794,664,125 | 117,346,288,570 | 68,592,423 | 37.4017X | 88.31% |
| | NKTL19 | 760,949,646 | 758,760,965 | 112,302,728,877 | 41,135,826 | 35.7942X | 88.12% |
| | NKTL2 | 1,622,184,516 | 1,606,682,412 | 235,112,782,372 | 113,642,153 | 74.9374X | 91.97% |
| | NKTL20 | 1,285,261,763 | 1,276,099,179 | 187,580,981,824 | 116,528,202 | 59.7876X | 91.77% |
| | NKTL21 | 836,562,841 | 833,648,713 | 124,196,105,527 | 208,112,821 | 39.585X | 89.27% |
| | NKTL22 | 803,210,131 | 799,261,692 | 118,967,511,784 | 198,121,640 | 37.9185X | 88.71% |
| | NKTL23 | 750,291,419 | 747,357,984 | 111,278,286,066 | 172,518,415 | 35.4677X | 87.84% |
| | NKTL24 | 761,633,690 | 758,183,972 | 112,900,581,108 | 185,055,987 | 35.9848X | 90.25% |
| | NKTL25 | 1,623,436,934 | 1,610,764,801 | 238,862,855,285 | 256,904,276 | 76.1327X | 92.05% |
| | NKTL26 | 712,553,555 | 709,714,472 | 89,482,905,017 | 103,946,862 | 28.5209X | 82.18% |
| | NKTL27 | 523,598,128 | 459,459,352 | 57,372,191,688 | 55,674,681 | 18.2862X | 39.39% |
| | NKTL28 | 706,818,570 | 706,319,632 | 92,224,970,337 | 84,116,401 | 29.3948X | 84.50% |
| | NKTL29 | 701,960,783 | 701,168,353 | 92,202,504,306 | 79,013,510 | 29.3877X | 84.75% |
| | NKTL3 | 1,513,174,471 | 1,505,833,679 | 148,809,168,842 | 49,447,396 | 47.4299X | 89.82% |
| | NKTL30 | 644,198,391 | 643,396,676 | 84,801,435,813 | 71,928,890 | 27.0287X | 81.61% |
| | NKTL31 | 939,968,348 | 937,273,257 | 118,112,219,219 | 144,057,256 | 37.6459X | 88.79% |
| | NKTL4 | 1,506,517,725 | 1,501,202,296 | 150,057,353,436 | 43,342,311 | 47.8277X | 90.56% |
| | NKTL5 | 715,852,632 | 713,425,433 | 106,204,276,606 | 153,585,810 | 33.8505X | 86.94% |
| | NKTL6 | 769,522,280 | 766,826,335 | 114,310,390,514 | 177,352,365 | 36.4341X | 88.35% |
| | NKTL7 | 1,781,494,494 | 1,771,453,412 | 260,759,611,987 | 103,097,630 | 83.1118X | 91.40% |
| | NKTL8 | 1,446,354,107 | 1,437,116,470 | 210,952,211,424 | 85,405,433 | 67.2367X | 91.26% |
| | NKTL9 | 1,863,037,075 | 1,853,201,145 | 275,886,547,386 | 371,079,849 | 87.9332X | 91.98% |
| | NKTL34 | 755,570,667 | 752,092,358 | 102,968,185,235 | 54,073,193 | 32.819X | 89.72% |
| | NKTL35 | 747,351,130 | 741,135,364 | 100,580,548,103 | 59,464,340 | 32.058X | 89.46% |
| | NKTL36 | 594,619,201 | 591,623,728 | 81,822,248,264 | 37,639,569 | 26.0792X | 80.17% |
| | NKTL37 | 678,868,697 | 675,770,511 | 89,769,035,386 | 67,421,746 | 28.6121X | 85.31% |
| | NKTL38 | 806,844,127 | 802,530,695 | 105,059,281,087 | 91,238,025 | 33.4855X | 86.58% |
| | NKTL39 | 792,625,038 | 788,513,419 | 103,701,836,047 | 85,345,394 | 33.0529X | 84.45% |
| | NKTL40 | 491,724,834 | 489,955,462 | 67,086,939,964 | 36,869,367 | 21.3826X | 61.98% |
| | NKTL41 | 796,740,991 | 794,362,605 | 105,431,245,097 | 78,926,309 | 33.6041X | 75.41% |
| | NKTL42 | 818,981,399 | 814,750,001 | 108,001,322,416 | 84,247,168 | 34.4232X | 87.59% |

### Genomic DNA Extraction

Genomic DNA from snap frozen and formalin-fixed paraffin-embedded (FFPE) tumour tissues, and whole blood was extracted as previously described. Buccal swab genomic DNA was purified using E.Z.N.A. Tissue DNA Kit (Omega Bio-tek) according to manufacturer's instructions. The quality and quantity were assessed as described elsewhere.

### NK-Cell Isolation and Activation

Resting and Activated NK-cells were used as baseline to compare the relative expressions of PD-L1 in the tumours samples. NK-cell isolation was performed using human apheresis cone blood obtained from the Health Sciences Authority of Singapore. Peripheral blood mononuclear cells were acquired by density centrifugation at 400×g for 30 minutes using Ficoll-Paque Plus (GE Healthcare). NK-cells were isolated using EasySep Human NK Cell Isolation Kit (STEMCELL Technologies) according to the manufacturer's protocol. The purity of NK-cells was greater than 90% as determined by CD3- and CD56+ expression by flow cytometry.

The isolated cells were suspended in X-VIVO 15 medium (Lonza) supplemented with 5% heat-inactivated human serum (Innova Biosciences) with or without 200 U/ml IL-2 (Proleukin). 1×10⁶ cells were seeded on a 48-well plate and the activation of NK-cells was determined after 48 hours by flow cytometry as up-regulation of CD25-FITC (clone: M-A251; BD Biosciences) and CD69-BV421 (clone: FN50; BioLegend).

NK-cell isolation was performed using human apheresis cone blood obtained from the Health Sciences Authority of Singapore. Peripheral blood mononuclear cells were acquired by density centrifugation at 400×g for 30 minutes using Ficoll-Paque Plus (GE Healthcare). Removal of platelets was performed by slow centrifugation at 120×g for 10 minutes. NK-cells were isolated using EasySep Human NK Cell Isolation Kit (STEMCELL Technologies) according to the manufacturer's protocol with the starting cell concentration of 1×10⁸ cells/ml.

The isolated NK-cells were stained with Live/Dead Aqua viability dye (ThermoFisher Scientific) followed by surface staining with monoclonal antibodies specific for CD3-V500 (clone: UCHT1; BD Biosciences) and CD56-PeCy7 (clone: B159; BD Biosciences) to determine the efficiency of the isolation. The purity of NK-cells was greater than 90% as determined by CD3-CD56+ expression by flow cytometry.

The isolated cells were resuspended in X-VIVO 15 medium (Lonza) supplemented with 5% heat-inactivated human serum (Innova Biosciences) with or without 200 U/ml IL-2 (Proleukin). 1×10⁶ cells were seeded on a 48-well plate and the activation of NK-cells was determined after 48 hours by flow cytometry as up-regulation of CD25-FITC (clone: M-A251; BD Biosciences) and CD69-BV421 (clone: FN50; BioLegend).

### Whole Genome Sequencing

All sequencing libraries were prepared using TruSeq Nano DNA Library Prep Kit (Illumina). Paired-end sequencing was performed on HiSeq 2000 or HiSeq X Ten System (Illumina) as 2×101 bp or 2×151 bp, respectively. Due to high fragmentation of genomic DNA from FFPE material, a size selection step was conducted prior to library preparation for the FFPE tumour samples. Amplifiable DNA fragments of −200 bp from the FFPE samples are used for sequencing library construction to avoid false-negatives confidently in the discovery for SR within the PD-L1 gene.

Alternatively, for extranodal natural killer/T-cell lymphoma, whole-genome sequencing (WGS) was performed for all 40 pairs of tumours-normal samples described in this study. All sequencing libraries were prepared using TruSeq Nano DNA Library Prep Kit (Illumina). Due to high fragmentation of genomic DNA in FFPE material, a size selection step was conducted prior to library preparation for the FFPE tumours samples. Paired-end sequencing was performed on HiSeq 2000 or HiSeq X Ten System (Illumina) as 2×101 bp or 2×151 bp, respectively. The mean WGS data coverages for the tumours and normal are 68.9× and 42.2×, respectively.

### Whole-Transcriptome Sequencing

RNA extraction, and quality and quantity assessment were done as previously described. Sequencing libraries were prepared using the TruSeq Stranded Total RNA Library Prep Kit with Ribo-Zero (Illumina) and whole-transcriptome sequencing (WTS) was performed on HiSeq 2500, HiSeq 3000 or HiSeq X Ten System (Illumina) with 2×101 bp, 2×151 bp or 2×151 bp read length, respectively.

### Quantification and Normalization of RNA Transcripts

RNA reads were aligned using STAR to a combined reference of hs37d5 and EBV-1 in a 2-pass mode. The gene counts were normalized by DESeq2 and the significance in differential expression was calculated using two-tailed analysed rank-sum test. Statistical significance was considered as p<0.05.

cDNA Synthesis and Real-Time

Reverse transcription was performed for samples with available RNA using SuperScript III Reverse Transcriptase (Invitrogen).

### Whole Genome and Whole Transcriptome Sequencing

For extranodal natural killer/T-cell lymphoma, to generate WGS data from the extranodal natural killer/T-cell lymphoma specimen for this study, genomic DNA from snap frozen and FFPE tumours tissues, and whole blood was extracted as previously described. Buccal swab genomic DNA was purified using E.Z.N.A. Tissue DNA Kit (Omega Bio-tek) according to manufacturer's instructions. The quality and quantity were assessed as described elsewhere. Whole-genome sequencing was performed for all the tumours and, whole blood or buccal swab samples described in this study. All sequencing libraries were prepared using TruSeq Nano DNA Library Prep Kit (Illumina). A size selection step was conducted prior to library preparation for the FFPE tumours samples. RNA extraction, and quality and quantity assessment were done as previously described 2. Sequencing libraries were prepared using the TruSeq Stranded Total RNA Library Prep Kit with Ribo-Zero (Illumina).

### Detection and Filtering of Somatic Variants

Sequencing reads were aligned using BWA-MEM to the hs37d5 human reference genome. Strelka2 and MuSE were used to detect somatic short variants. Short variants were subsequently annotated by wAnnovar.

### Genomic Analysis of Structural Rearrangements

Prior to all downstream analysis, gDNA sequencing reads were aligned using BWA-MEM to the hs37d5 human reference genome and PCR duplicates were marked by Sambamba. To identify somatic structural rearrangements (SR), Manta was applied on the aligned gDNA reads of tumours-blood paired samples. All predicted SRs within the genic region of PD-L1 were verified with Sanger Sequencing. To determine if the predicted SRs from the gDNA sequencing data yielded transcript products, cDNA was obtained from the available corresponding RNA using SuperScript III Reverse Transcriptase (Invitrogen) for PCR-based validation and Sanger sequencing.

### Detection and Filtering of Structural Variations

DNA reads were aligned using BWA-MEM to the hs37d5 human reference genome and PCR duplicates were removed by Sambamba. Read pairs were marked as discordant if they did not align to the reference genome with the expected orientation and/or within the expected insert size. Reads were flagged as clipped when either end of the read did not match the reference genome.

Detection of somatic structural rearrangements (SR) was done by Manta and each candidate SR was subjected to the following filtering criteria: 1) SR is supported by at least 3 discordant read-pairs and at least 3 soft-clipped reads, and the sum of all supporting reads is at least 10; 2) zero discordant and soft-clipped reads present in the matching normal sample; 3) at least 20× coverage in both tumours and matching normal sample; and 4) SR is at least 1000 bp in size.

The histogram of unique samples having SR within a genomic region, i.e. the SR landscape, was tabulated using a 1 Mbp averaging sliding window in steps of 100 kbp along the main chromosomes of hs37d5. The breakpoints of putative SRs were converted to the BEDPE format and, together with the SR landscape, visualized as links using CIRCOS.

### Detection of Somatic Variations

WGS data was nalysed using FreeBayes 6 (-X -u -C5 -m30 -q20) and variants with score less than 30 were filtered out. Single nucleotide variants are predicted to be somatic only if it is called from the tumours and not the matching normal data.

### Detection of Somatic Single Nucleotide Variants and Indels

Somatic single nucleotide variants and indels in WGS data were called using FreeBayes. Candidate variants with a score of less than 30 were filtered away. Variants were predicted to be somatic only if it was called from the tumours and not the matching normal data.

### Analysis of Tumour Clonality

SciClone was used to analyse the clonality architecture of the tumours. CANVAS was used to analyse copy number and loss of heterozygousity information for each tumour, which were used as input for the clonality analysis by SciClone.

### PCR and Sanger Sequencing

For relapsed or refractory (RR) natural-killer/T-cell lymphoma, details about PCRconditions and sequencing were previously described. Primers were designed using Primer3 software and the sequences are listed in Table 6 for the discovery cohort ofthe 11 pembrolizumab-treated NKTL patients. Sanger sequences were aligned to hs37 reference genome and confirmed with BLAT. Alternatively, the primer sequences are also listed in Table 7 for the prevalence cohort of the 32 NKTL patients who were not subsequently treated with pembrolizumab.

TABLE 8
Primer-pairs used for the validation of PD-L1
structural rearrangement and JAK3-activating in
the discovery cohort of patients who were
subsequently treated with pembrolizumab.

| | | | | Forward | | Reverse |
|---|---|---|---|---|---|---|
| | | | | Primer | | Primer |
| | Event | Experi- | Seq | Sequence | Seq | Sequence |
| No. | Name | ment | ID | (5′ → -3′) | ID | (5′ → 3′) |
| 1 | NKTL | gDNA | 1 | ACATAAATA | 2 | GAGGCTCCT |
| | 1- | SR left | | CTGTCCCGT | | TGTTCAGAA |
| | in- | break- | | TCCA | | GT |
| | version | point | | | | |
| | | vali- | | | | |
| | | dation | | | | |
| 2 | NKTL | gDNA | 3 | TGTCACAGG | 4 | CAACCACAC |
| | 1- | SR right | | CGTCGATGA | | TCACATGAC |
| | in- | break- | | G | | AAGA |
| | version | point | | | | |
| | | vali- | | | | |
| | | dation | | | | |
| 3 | NKTL | gDNA | 5 | CAGATACAC | 6 | CTTTGGCCC |
| | 26- | SR | | ATTTGGAGG | | TGTTTGTGT |
| | dupli- | vali- | | AGACG | | CC |
| | cation | dation | | | | |
| 4 | NKTL | gDNA | 7 | ATTCAAGTT | 8 | AAGACTTTT |
| | 28- | SR | | TCCTTTCCA | | GGTTGGTAT |
| | trans- | vali- | | GAAGCA | | TTTCTGT |
| | location | dation | | | | |
| 5 | NKTL | gDNA | 9 | CCATGCACG | 10 | TCAGTATCT |
| | 31- | SR | | GTATCTCAT | | CATCCCACC |
| | trans- | vali- | | TTAAT | | TGAC |
| | location | dation | | | | |
| 6 | NKTL | gDNA | 11 | GGGGCTCTC | 12 | AAGAAACCC |
| | 29- | SNV | | ACTGTCTCC | | ACGCATCTT |
| | JAK3- | vali- | | A | | CTCT |
| | missense | dation | | | | |
| 7 | NKTL | gDNA | 13 | GGGGCTCTC | 14 | AAGAAACCC |
| | 30- | SNV | | ACTGTCTCC | | ACGCATCTT |
| | JAK3- | vali- | | A | | CTCT |
| | missense | dation | | | | |
| 8 | NKTL | gDNA | 15 | CATGTGCTG | 16 | CCTCTTCCT |
| | 27- | in- | | TGACTGCTT | | ACAGTACTC |
| | ARID | sertion | | GT | | CCC |
| | 1B- | vali- | | | | |
| | in- | dation | | | | |
| | sertion | | | | | |

TABLE 9
Primer-pairs used for the validation of PD-L1 structural rearrangement
in the prevalence cohort of patients who were not
subsequently treated with pembrolizumab.

| | Event | | Seq | Forward Primer Sequence | Seq | Reverse Primer Sequence |
|---|---|---|---|---|---|---|
| No. | Name | Experiment | ID | (5′ → 3′) | ID | (5′ → 3′) |
| 1 | NKTL | gDNA SR | 1 | ACATAAATACTGTCCCGTTC | 2 | GAGGCTCCTTGTTCA |
| | 1- | left | | CA | | GAAGT |
| | inversion | breakpoint | | | | |
| | | validation | | | | |
| 2 | NKTL | gDNA SR | 3 | TGTCACAGGCGTCGATGAG | 4 | CAACCACACTCACAT |
| | 1- | right | | | | GACAAGA |
| | inversion | breakpoint | | | | |
| | | validation | | | | |
| 3 | NKTL | gDNA SR | 17 | CAAGTTTCATTCTGTGGCCCA | 18 | TTGGGTCAAAGCGGA |
| | 4- | validation | | | | ATGTG |
| | deletion | | | | | |
| 4 | NKTL | gDNA SR | 19 | CAGATACACATTTGGAGGAG | 20 | ATGGATAGGGCTGCA |
| | 6- | validation | | ACG | | GGTGA |
| | complex | | | | | |
| 5 | NKTL | gDNA SR | 21 | GCTCATCCTAGGAAGACGGG | 22 | AACTGGAGTCGAAG |
| | 11- | validation | | | | GTCACA |
| | duplication | | | | | |
| 6 | NKTL | gDNA SR | 23 | GAGAAAACAGAGGGTCAAG | 24 | GAAACCAAAAGCAA |
| | 15- | validation | | AAGAT | | GCAGGAGTAG |
| | duplication | | | | | |
| 7 | NKTL | gDNA SR | 25 | TCCCTGACAATTCTAAATCG | 26 | ACCCGACTTAACCTC |
| | 16- | validation | | AGT | | TGCAA |
| | translocation | | | | | |
| 8 | NKTL | gDNA SR | 27 | GCCCGTCATTTTTCAGTTGCA | 28 | CAGGAGAATGGCGT |
| | 17- | validation | | | | GAACTC |
| | deletion | | | | | |
| 9 | NKTL | gDNA SR | 5 | CAGATACACATTTGGAGGAG | 6 | CTTTGGCCCTGTTTGT |
| | 26- | validation | | ACG | | GTCC |
| | duplication | | | | | |
| 10 | NKTL | gDNA SR | 7 | ATTCAAGTTTCCTTTCCAGA | 8 | AAGACTTTTGGTTGG |
| | 28- | validation | | AGCA | | TATTTTCTGT |
| | translocation | | | | | |
| 11 | NKTL | gDNA SR | 9 | CCATGCACGGTATCTCATTT | 10 | TCAGTATCTCATCCC |
| | 31- | validation | | AAT | | ACCTGAC |
| | translocation | | | | | |
| 12 | NKTL | gDNA SR | 29 | CCAGACCACTTCCCATGAAA | 30 | TACTCATACATTTGG |
| | 35- | validation | | TTAA | | CCTCAGTTG |
| | deletion | | | | | |
| 13 | NKTL | gDNA SR | 31 | TATACCAAGAGATCCAGTGA | 32 | AATCATGTTTCAGTA |
| | 37- | left | | TGGT | | CCATTGGCT |
| | inversion | breakpoint | | | | |
| | | validation | | | | |
| 14 | NKTL | gDNA SR | 33 | CTACTCTCCAGCCCATCTAT | 34 | ATCTATTGAGGGCTG |
| | 37- | right | | TGAG | | ATCTGGG |
| | inversion | breakpoint | | | | |
| | | validation | | | | |
| 15 | NKTL | cDNA SR | 35 | ACTTGGTAATTCTGGGAGCCA | 36 | CCACCTTCTGAACAG |
| | 4- | validation | | | | TGACC |
| | deletion | | | | | |
| 16 | NKTL | cDNA SR | 37 | ACTTGGTAATTCTGGGAGCCA | 38 | GGCCAGCTGAGGTCT |
| | 6- | validation | | | | TTATT |
| | complex | | | | | |
| 17 | NKTL | cDNA SR | 39 | CAACACAACAACTAATGAG | 40 | CCTCCCATGACATCT |
| | 15- | validation | | ATTTTCTACTG | | CTCCTCTTATG |
| | duplication | | | | | |
| 18 | NKTL | cDNA SR | 41 | AATGAAAGGACTCACTTGGT | 42 | GTTTTGCAGCTAGAA |
| | 16- | validation | | AATTCT | | TTCAGTTGTAA |
| | translocation | | | | | |
| 19 | NKTL | cDNA SR | 43 | CCTCCAAATGAAAGGACTCA | 44 | GCAGGTTTGGCAATT |
| | 17- | validation | | CT | | CTGATTC |
| | deletion | | | | | |
| 20 | NKTL | cDNA SR | 45 | CAGCATTGGAACTTCTGATC | 46 | GGCCTCAGTTGTCAC |
| | 35- | validation | | TTCA | | AGTATTTT |
| | deletion | | | | | |
| 21 | NKTL | cDNA SR | 47 | TGAAAGGACTCACTTGGTAA | 48 | AGGTATAATAATGCT |
| | 37- | validation | | TTCTG | | GCCTGAGAT |
| | inversion | | | | | |

### Histological Studies and Scoring

For relapsed or refractory (RR) natural-killer/T-cell lymphoma, PD-L1 IHC analysis was performed with anti-PD-L1 rabbit monoclonal antibody (SP263, Ventana). PD-L1 positivity was evaluated as a percentage of positively stained tumour cells at the cell membrane. Alternatively, for extranodal natural killer/T-cell lymphoma, PD-L expression was evaluated as staining at the cell membrane and scored based on the percentage of positive tumours cells and staining intensity. The following grading was used: 0, no staining, 1+, weak, 2+ mild and 3+ strong staining. The same pathologist reviewed all PD-L1 IHC stainings. Available H-scores for the samples used in this study is included as Table 10.

TABLE 10
Available H-scores for samples.

| | | PD-L1 Membrane | | PD-L1 | |
|---|---|---|---|---|---|
| | | Staining (% Positive | | 3′ UTR | Pembro- |
| | | Tumours Cells and | PD-L1 | Dis- | lizumab- |
| No. | Sample | Grade)^{a} | H-score | ruption | treated |
| 1 | NKTL1 | ^{b}100%, 3+ | 250 | Yes | Yes |
| 2 | NKTL2 | 15%, 2+ | 20 | No | No |
| 3 | NKTL3 | <1%, 1+ | 1 | No | No |
| 4 | NKTL4 | 55%, 3+ | 85 | Yes | No |
| 5 | NKTL5 | 30%, 2+ | 40 | No | No |
| 6 | NKTL6 | 50%, 2+ | 75 | Yes | No |
| 7 | NKTL7 | 55%, 2+ | 85 | No | No |
| 8 | NKTL8 | 50%, 2+ | 80 | No | No |
| 9 | NKTL9 | 15%, 2+ | 20 | No | No |
| 10 | NKTL10 | 40%, 2+ | 60 | No | No |
| 11 | NKTL11 | 100%, 3+ | 210 | Yes | No |
| 12 | NKTL12 | NA | NA | No | No |
| 13 | NKTL13 | 30%, 2+ | 60 | No | No |
| 14 | NKTL14 | NA | NA | No | No |
| 15 | NKTL15 | 25%, 3+ | 45 | Yes | No |
| 16 | NKTL16 | 80%, 3+ | 190 | Yes | No |
| 17 | NKTL17 | 80%, 3+ | 135 | Yes | No |
| 18 | NKTL18 | 30%, 2+ | 35 | No | No |
| 19 | NKTL19 | 60%, 2+ | 80 | No | No |
| 20 | NKTL20 | 90%, 2+ | 120 | No | No |
| 21 | NKTL21 | NA | NA | No | No |
| 22 | NKTL22 | 80%, 3+ | 210 | No | No |
| 23 | NKTL23 | 80%, 3+ | 200 | No | No |
| 24 | NKTL24 | 60%, 3+ | 120 | No | No |
| 25 | NKTL25 | ^{b}72%, 3+ | 126 | No | Yes |
| 26 | NKTL26 | ^{b}35%, 2+ | 40 | Yes | Yes |
| 27 | NKTL27 | 50%, 3+ | 85 | No | Yes |
| 28 | NKTL28 | 70%, 3+ | 190 | Yes | Yes |
| 29 | NKTL29 | 6%, 2+ | 7 | No | No |
| 30 | NKTL30 | 60%, 3+ | 120 | No | No |
| 31 | NKTL31 | 20%, 1+ | 20 | Yes | Yes |
| 34 | NKTL34 | NA | NA | No | No |
| 35 | NKTL35 | NA | NA | Yes | No |
| 36 | NKTL36 | NA | NA | No | No |
| 37 | NKTL37 | NA | NA | Yes | No |
| 38 | NKTL38 | NA | NA | No | No |
| 39 | NKTL39 | NA | NA | No | No |
| 40 | NKTL40 | NA | NA | No | No |
| 41 | NKTL41 | NA | NA | No | No |
| 42 | NKTL42 | NA | NA | No | No |

^{a}Assessed by immunohistochemistry.
^{b}Clinical Response Reported in Kwong et al. Blood 2017.

### Cell Lines and Constructs

K-562 and Jurkat cell lines was purchased from ATCC and NK-S1 was generated in-house. LGC Standards authenticated the K-562 and Jurkat cell lines. Jurkat cells were maintained in RPMI 1640 (Gibco) supplemented with 10% FBS (HyClone), and K-562 and NK-S1 were grown in DMEM (Gibco) supplemented with 10% FBS (HyClone), 10% horse serum (Gibco) and 2 mM L-glutamine (Gibco). The cells were grown at 37° C. in the presence of 5% CO₂ and routinely checked for mycoplasma contamination using MycoAlert Mycoplasma Detection Kit (Lonza).

For extranodal natural killer/T-cell lymphoma, K-562 and Jurkat cell lines from ATCC and in-house NK-S1 cell line was used to investigate the regulatory effect of the smallest structural rearrangement found within the 3′UTR of PD-L1 with the study cohort.

Wild type PD-L1 3′UTR (ENST00000381573.8) from SNK6 cell line was cloned into the XhoI and NotI sites of the psiCHECK-2 vector (Promega). For the partially inverted 3′UTR recapitulating the rearrangement identified in sample NKTL1, three individual pieces with overhangs were amplified from a wild type sample (SNK6) and ligated together by PCR. Cloning was performed using Q5 High-Fidelity 2× Master Mix (New England BioLabs). All cloning primers used to clone the full-length wild type and mutant PD-L1 3′UTR are described in Table 11.

TABLE 11
Cloning primers used for the cloning of the full-
length 3′ UTR of PD-L1 with and without the
micro-inversion of 206 bp long.

| For WT clone | | | |
|---|---|---|---|
| | Seq | 5′ → 3′ | |
| Primer name | ID | sequence | Explanation |
| CD274- | 49 | CGTAGTCTC | Tail-XhoI-CD274-3′ UTR |
| 3UTR_XhoI_F | | GAGTCCAGC | to prime start of 3′ |
| | | ATTGGAACT | UTR |
| | | TCTGA | |
| CD274- | 50 | CAATTAGCG | Tail-NotI-CD274-3′ UTR |
| 3UTR_ | | GCCGCAACT | to prime end of 3′ |
| NotI_R | | TTCTCCACT | UTR |
| | | GGGATGT | |
| For Inverted | | | |
| Clone | | | |
| To amplify | | | |
| fragment A | | 5′ → 3′ | |
| Primer name | | sequence | Explanation |
| CD274- | 51 | TCCAGCATT | CD274-3′ UTR to prime |
| 3UTR_F | | GGAACTTCT | start of 3′ UTR |
| | | GATCTTCAA | |
| | | G | |
| CD274-A-R | 52 | TGACTGAGA | Fragment A reverse |
| | | GTCTCAAGG | with fragment B 15 |
| | | TCTCCCTCC | nt overhang |
| | | AGGCTCCC | |
| To amplify | | | |
| fragment B | | | |
| (the inverted | | | |
| region) | | 5′ → 3′ | |
| Primer name | | sequence | Explanation |
| Inv-over- | 53 | CCTGGAGGG | Start of inversion |
| hang-F | | AGACCTTGA | (fragment B) forward |
| | | GACTCTCAG | with fragment A 15 |
| | | TCATGCAG | nt overhang |
| Inv-over- | 54 | GTCCCGTTC | End of inversion |
| hang-R | | CAACACTGA | (fragment B) reverse |
| | | TACTTTCAA | with fragment C 15 |
| | | ATGCCTGA | nt overhang |
| To amplify | | | |
| fragment C | | 5′ → 3′ | |
| Primer name | | sequence | Exaplanation |
| CD274-C-F | 55 | CATTTGAAA | Fragment C forward |
| and F2 | | GTATCAGTG | with fragment B 15 |
| | | TTGGAACGG | nt overhang |
| | | GACAGTAT | |
| CD274- | 56 | AACTTTCTC | CD274-3′ UTR to prime |
| 3UTR_R | | CACTGGGAT | end of 3′ UTR |
| | | GTTAAACTG | |
| To merge | | | |
| fragment | | | |
| A & B | | 5′ → 3′ | |
| Primer name | | sequence | Explanation |
| CD274- | 57 | TCCAGCATT | CD274-3′ UTR to prime |
| 3UTR_F | | GGAACTTCT | start of 3′ UTR |
| | | GATCTTCAA | |
| | | G | |
| Inv-over- | 58 | GTCCCGTTC | End of inversion |
| hang-R | | CAACACTGA | (fragment B) reverse |
| | | TACTTTCAA | with fragment C 15 |
| | | ATGCCTGA | nt overhang |
| To merge | | | |
| fragment | | | |
| AB & C | | 5′ → 3′ | |
| Primer name | | sequence | Explanation |
| CD274- | 59 | CGTAGTCTC | Tail-XhoI-CD274-3′ UTR |
| 3UTR_XhoI_F | | GAGTCCAGC | to prime start of 3′ |
| | | ATTGGAACT | UTR |
| | | TCTGA | |
| CD274- | 60 | CAATTAGCG | Tail-NotI-CD274-3′ UTR |
| 3UTR_ | | GCCGCAACT | to prime end of 3′ |
| NotI_R | | TTCTCCACT | UTR |
| | | GGGATGT | |

### Transfection and Luciferase Assay

For relapsed or refractory (RR) natural-killer/T-cell lymphoma, for K-562 and Jurkat, 5×10⁴ cells and 6×10⁴ cells were seeded on a 48-well plate in triplicates, respectively, and transfected with 250 ng plasmid DNA using the Lipofectamine 3000 Reagent (Invitrogen). For NK-S1 cells, 2×10⁵ cells were electroporated in triplicates on a 24-well plate with 1 μg plasmid DNA using the Neon Transfection System (Invitrogen). The pulse parameters used were the following: voltage 1300, width 10 and no. 3. Alternatively, for extranodal natural killer/T-cell lymphoma, for K-562 cells, 2.5×10⁵ cells were seeded on a 48-well plate in triplicates and transfected with 250 ng plasmid DNA using the Lipofectamine 3000 Reagent (Invitrogen). For NK-S1 and Jurkat cells, 2.5×10⁵ cells were electroporated in triplicates on a 24-well plate with 1 μg plasmid DNA using the Neon Transfection System (Invitrogen).

The cells were lysed with Passive Lysis Buffer (Promega) after 48 hours. Luminescence was measured using the Dual-Luciferase Reporter Assay System (Promega) and the GloMax-Multi+ Detection System (Promega). Renilla luciferase activities were divided by Firefly luciferase activities and the results were normalized to the empty vector control (mock). Statistical significance was calculated by two-sided t-test. Statistical significance was considered as P<0.05. All experiments were repeated at least twice.

### Data Availability

The WGS data of 43 natural killer/T-cell lymphoma (NKTL)-normal/blood pairs and whole transcriptomic sequencing (WTS) data of 28 NKTL have been deposited in European Genome Archive (EGA) under the study accession code: EGAS00001002420.

TABLE 12
Additional sequences

| SEQ | | |
|---|---|---|
| ID | | |
| NO | Description | Sequence |
| 61 | NKTL1 | GGCATTTGAAAGTATCA*GTGTTGGAACGGGACAG |
| | validated | |
| | Sanger | |
| | sequence | |
| 62 | NKTL11 | TGTCATGTGAGTGTGGTTGT*GAACAGTTCCTGAACTCTGA |
| | validated | |
| | Sanger | |
| | sequence | |
| 63 | NKTL15 | TAAGAAGAAAGTTATATTAT*AATATAGTTTGCTTTTACAA |
| | validated | |
| | Sanger | |
| | sequence | |
| 64 | NKTL6 | AGCGTGACAAGAGGAAGGAA*TGTGCCACCATGCCCAGCTA |
| | (complex | |
| | case) 1 | |
| | validated | |
| | Sanger | |
| | sequence | |
| 65 | NKTL6 | CGTATTGGCCAGGATAGTCT*AGAAAATTTTGCTAAAGCAG |
| | (complex | |
| | case) 2 | |
| | validated | |
| | Sanger | |
| | sequence | |
| 66 | NKTL4 | TGTGTTGTAAAGCTAAGTAG*CTCAGGTACTTTGCTATCCC |
| | validated | |
| | Sanger | |
| | sequence | |
| 67 | NKTL17 | CATTTAAGATGAGTCAGAGT*TTTTTGAGACGGAGTCTCGC |
| | validated | |
| | Sanger | |
| | sequence | |
| 68 | NKTL16 | CAGGAGAATGGGTATGGATG*AGAACACATACTTCCTCTCC |
| | validated | |
| | Sanger | |
| | sequence | |
| 69 | NKTL26 | CTGATCTTCAAGCAGGGGATT*GATGTGCTTTGTTAAACAGA |
| | validated | |
| | Sanger | |
| | sequence | |
| 70 | NKTL28 | ATGTTAAAAGCACGTATTTT*GAATAAAATGTTACTTTGTC |
| | validated | |
| | Sanger | |
| | sequence | |
| 71 | NKTL31 | CTCCCTCCCTTTCTCTCTCT*CTCTCTCTCTTTGGTAATGG |
| | validated | |
| | Sanger | |
| | sequence | |
| 72 | FLN375 | CGTGGGATGCAGGCAATGTG*GAATATAACAAATAAAGCAA |
| | validated | |
| | Sanger | |
| | sequence | |
| 73 | FLN377 | AATATGGAAGGGGATTCCAA*ATCTGAAGGGACCTCAGGGG |
| | validated | |
| | Sanger | |
| | sequence | |
| 74 | JAK3 | ATGGCACCTCCAAGTGAAGAGACGCCCCTGATCCCTCAGCGTTCATGCA |
| | cDNA wild | GCCTCTTGTCCACGGAGGCTGGTGCCCTGCATGTGCTGCTGCCCGCTCG |
| | type | GGGCCCCGGGCCCCCCCAGCGCCTATCTTTCTCCTTTGGGGACCACTTG |
| | | GCTGAGGACCTGTGCGTGCAGGCTGCCAAGGCCAGCGGCATCCTGCCTG |
| | | TGTACCACTCCCTCTTTGCTCTGGCCACGGAGGACCTGTCCTGCTGGTTC |
| | | CCCCCGAGCCACATCTTCTCCGTGGAGGATGCCAGCACCCAAGTCCTGC |
| | | TGTACAGGATTCGCTTTTACTTCCCCAATTGGTTTGGGCTGGAGAAGTG |
| | | CCACCGCTTCGGGCTACGCAAGGATTTGGCCAGTGCTATCCTTGACCTG |
| | | CCAGTCCTGGAGCACCTCTTTGCCCAGCACCGCAGTGACCTGGTGAGTG |
| | | GGCGCCTCCCCGTGGGCCTCAGTCTCAAGGAGCAGGGTGAGTGTCTCAG |
| | | CCTGGCCGTGTTGGACCTGGCCCGGATGGCGCGAGAGCAGGCCCAGCG |
| | | GCCGGGAGAGCTGCTGAAGACTGTCAGCTACAAGGCCTGCCTACCCCC |
| | | AAGCCTGCGCGACCTGATCCAGGGCCTGAGCTTCGTGACGCGGAGGCG |
| | | TATTCGGAGGACGGTGCGCAGAGCCCTGCGCCGCGTGGCCGCCTGCCA |
| | | GGCAGACCGGCACTCGCTCATGGCCAAGTACATCATGGACCTGGAGCG |
| | | GCTGGATCCAGCCGGGGCCGCCGAGACCTTCCACGTGGGCCTCCCTGGG |
| | | GCCCTTGGTGGCCACGACGGGCTGGGGCTGCTCCGCGTGGCTGGTGACG |
| | | GCGGCATCGCCTGGACCCAGGGAGAACAGGAGGTCCTCCAGCCCTTCT |
| | | GCGACTTTCCAGAAATCGTAGACATTAGCATCAAGCAGGCCCCGCGCGT |
| | | TGGCCCGGCCGGAGAGCACCGCCTGGTCACTGTTACCAGGACAGACAA |
| | | CCAGATTTTAGAGGCCGAGTTCCCAGGGCTGCCCGAGGCTCTGTCGTTC |
| | | GTGGCGCTCGTGGACGGCTACTTCCGGCTGACCACGGACTCCCAGCACT |
| | | TCTTCTGCAAGGAGGTGGCACCGCCGAGGCTGCTGGAGGAAGTGGCCG |
| | | AGCAGTGCCACGGCCCCATCACTCTGGACTTTGCCATCAACAAGCTCAA |
| | | GACTGGGGGCTCACGTCCTGGCTCCTATGTTCTCCGCCGCAGCCCCCAG |
| | | GACTTTGACAGCTTCCTCCTCACTGTCTGTGTCCAGAACCCCCTTGGTCC |
| | | TGATTATAAGGGCTGCCTCATCCGGCGCAGCCCCACAGGAACCTTCCTT |
| | | CTGGTTGGCCTCAGCCGACCCCACAGCAGTCTTCGAGAGCTCCTGGCAA |
| | | CCTGCTGGGATGGGGGGCTGCACGTAGATGGGGTGGCAGTGACCCTCA |
| | | CTTCCTGCTGTATCCCCAGACCCAAAGAAAAGTCCAACCTGATCGTGGT |
| | | CCAGAGAGGTCACAGCCCACCCACATCATCCTTGGTTCAGCCCCAATCC |
| | | CAATACCAGCTGAGTCAGATGACATTTCACAAGATCCCTGCTGACAGCC |
| | | TGGAGTGGCATGAGAACCTGGGCCATGGGTCCTTCACCAAGATTTACCG |
| | | GGGCTGTCGCCATGAGGTGGTGGATGGGGAGGCCCGAAAGACAGAGGT |
| | | GCTGCTGAAGGTCATGGATGCCAAGCACAAGAACTGCATGGAGTCATT |
| | | CCTGGAAGCAGCGAGCTTGATGAGCCAAGTGTCGTACCGGCATCTCGTG |
| | | CTGCTCCACGGCGTGTGCATGGCTGGAGACAGCACCATGGTGCAGGAA |
| | | TTTGTACACCTGGGGGCCATAGACATGTATCTGCGAAAACGTGGCCACC |
| | | TGGTGCCAGCCAGCTGGAAGCTGCAGGTGGTCAAACAGCTGGCCTACG |
| | | CCCTCAACTATCTGGAGGACAAAGGCCTGCCCCATGGCAATGTCTCTGC |
| | | CCGGAAGGTGCTCCTGGCTCGGGAGGGGGCTGATGGGAGCCCGCCCTT |
| | | CATCAAGCTGAGTGACCCTGGGGTCAGCCCCGCTGTGTTAAGCCTGGAG |
| | | ATGCTCACCGACAGGATCCCCTGGGTGGCCCCCGAGTGTCTCCGGGAGG |
| | | CGCAGACACTTAGCTTGGAAGCTGACAAGTGGGGCTTCGGCGCCACGG |
| | | TCTGGGAAGTGTTTAGTGGCGTCACCATGCCCATCAGTGCCCTGGATCC |
| | | TGCTAAGAAACTCCAATTTTATGAGGACCGGCAGCAGCTGCCGGCCCCC |
| | | AAGTGGACAGAGCTGGCCCTGCTGATTCAACAGTGCATGGCCTATGAGC |
| | | CGGTCCAGAGGCCCTCCTTCCGAGCCGTCATTCGTGACCTCAATAGCCT |
| | | CATCTCTTCAGACTATGAGCTCCTCTCAGACCCCACACCTGGTGCCCTG |
| | | GCACCTCGTGATGGGCTGTGGAATGGTGCCCAGCTCTATGCCTGCCAAG |
| | | ACCCCACGATCTTCGAGGAGAGACACCTCAAGTACATCTCACAGCTGGG |
| | | CAAGGGCAACTTTGGCAGCGTGGAGCTGTGCCGCTATGACCCGCTAGGC |
| | | GACAATACAGGTGCCCTGGTGGCCGTGAAACAGCTGCAGCACAGCGGG |
| | | CCAGACCAGCAGAGGGACTTTCAGCGGGAGATTCAGATCCTCAAAGCA |
| | | CTGCACAGTGATTTCATTGTCAAGTATCGTGGTGTCAGCTATGGCCCGG |
| | | GCCGCCAGAGCCTGCGGCTGGTCATGGAGTACCTGCCCAGCGGCTGCTT |
| | | GCGCGACTTCCTGCAGCGGCACCGCGCGCGCCTCGATGCCAGCCGCCTC |
| | | CTTCTCTATTCCTCGCAGATCTGCAAGGGCATGGAGTACCTGGGCTCCC |
| | | GCCGCTGCGTGCACCGCGACCTGGCCGCCCGAAACATCCTCGTGGAGA |
| | | GCGAGGCACACGTCAAGATCGCTGACTTCGGCCTAGCTAAGCTGCTGCC |
| | | GCTTGACAAAGACTACTACGTGGTCCGCGAGCCAGGCCAGAGCCCCATT |
| | | TTCTGGTATGCCCCCGAATCCCTCTCGGACAACATCTTCTCTCGCCAGTC |
| | | AGACGTCTGGAGCTTCGGGGTCGTCCTGTACGAGCTCTTCACCTACTGC |
| | | GACAAAAGCTGCAGCCCCTCGGCCGAGTTCCTGCGGATGATGGGATGT |
| | | GAGCGGGATGTCCCCGCCCTCTGCCGCCTCTTGGAACTGCTGGAGGAGG |
| | | GCCAGAGGCTGCCGGCGCCTCCTGCCTGCCCTGCTGAGGTTCACGAGCT |
| | | CATGAAGCTGTGCTGGGCCCCTAGCCCACAGGACCGGCCATCATTCAGC |
| | | GCCCTGGGCCCCCAGCTGGACATGCTGTGGAGCGGAAGCCGGGGGTGT |
| | | GAGACTCATGCCTTCACTGCTCACCCAGAGGGCAAACACCACTCCCTGT |
| | | CCTTTTCATAG |
| 75 | JAK3 | ATGGCACCTCCAAGTGAAGAGACGCCCCTGATCCCTCAGCGTTCATGCA |
| | cDNA | GCCTCTTGTCCACGGAGGCTGGTGCCCTGCATGTGCTGCTGCCCGCTCG |
| | single | GGGCCCCGGGCCCCCCCAGCGCCTATCTTTCTCCTTTGGGGACCACTTG |
| | mutation 1 | GCTGAGGACCTGTGCGTGCAGGCTGCCAAGGCCAGCGGCATCCTGCCTG |
| | | TGTACCACTCCCTCTTTGCTCTGGCCACGGAGGACCTGTCCTGCTGGTTC |
| | | CCCCCGAGCCACATCTTCTCCGTGGAGGATGCCAGCACCCAAGTCCTGC |
| | | TGTACAGGATTCGCTTTTACTTCCCCAATTGGTTTGGGCTGGAGAAGTG |
| | | CCACCGCTTCGGGCTACGCAAGGATTTGGCCAGTGCTATCCTTGACCTG |
| | | CCAGTCCTGGAGCACCTCTTTGCCCAGCACCGCAGTGACCTGGTGAGTG |
| | | GGCGCCTCCCCGTGGGCCTCAGTCTCAAGGAGCAGGGTGAGTGTCTCAG |
| | | CCTGGCCGTGTTGGACCTGGCCCGGATGGCGCGAGAGCAGGCCCAGCG |
| | | GCCGGGAGAGCTGCTGAAGACTGTCAGCTACAAGGCCTGCCTACCCCC |
| | | AAGCCTGCGCGACCTGATCCAGGGCCTGAGCTTCGTGACGCGGAGGCG |
| | | TATTCGGAGGACGGTGCGCAGAGCCCTGCGCCGCGTGGCCGCCTGCCA |
| | | GGCAGACCGGCACTCGCTCATGGCCAAGTACATCATGGACCTGGAGCG |
| | | GCTGGATCCAGCCGGGGCCGCCGAGACCTTCCACGTGGGCCTCCCTGGG |
| | | GCCCTTGGTGGCCACGACGGGCTGGGGCTGCTCCGCGTGGCTGGTGACG |
| | | GCGGCATCGCCTGGACCCAGGGAGAACAGGAGGTCCTCCAGCCCTTCT |
| | | GCGACTTTCCAGAAATCGTAGACATTAGCATCAAGCAGGCCCCGCGCGT |
| | | TGGCCCGGCCGGAGAGCACCGCCTGGTCACTGTTACCAGGACAGACAA |
| | | CCAGATTTTAGAGGCCGAGTTCCCAGGGCTGCCCGAGGCTCTGTCGTTC |
| | | GTGGCGCTCGTGGACGGCTACTTCCGGCTGACCACGGACTCCCAGCACT |
| | | TCTTCTGCAAGGAGGTGGCACCGCCGAGGCTGCTGGAGGAAGTGGCCG |
| | | AGCAGTGCCACGGCCCCATCACTCTGGACTTTGCCATCAACAAGCTCAA |
| | | GACTGGGGGCTCACGTCCTGGCTCCTATGTTCTCCGCCGCAGCCCCCAG |
| | | GACTTTGACAGCTTCCTCCTCACTGTCTGTGTCCAGAACCCCCTTGGTCC |
| | | TGATTATAAGGGCTGCCTCATCCGGCGCAGCCCCACAGGAACCTTCCTT |
| | | CTGGTTGGCCTCAGCCGACCCCACAGCAGTCTTCGAGAGCTCCTGGCAA |
| | | CCTGCTGGGATGGGGGGCTGCACGTAGATGGGGTGGCAGTGACCCTCA |
| | | CTTCCTGCTGTATCCCCAGACCCAAAGAAAAGTCCAACCTGATCGTGGT |
| | | CCAGAGAGGTCACAGCCCACCCACATCATCCTTGGTTCAGCCCCAATCC |
| | | CAATACCAGCTGAGTCAGATGACATTTCACAAGATCCCTGCTGACAGCC |
| | | TGGAGTGGCATGAGAACCTGGGCCATGGGTCCTTCACCAAGATTTACCG |
| | | GGGCTGTCGCCATGAGGTGGTGGATGGGGAGGCCCGAAAGACAGAGGT |
| | | GCTGCTGAAGGTCATGGATGCCAAGCACAAGAACTGCATGGAGTCATT |
| | | CCTGGAAG[C > T,p.A573V]AGCGAGCTTGATGAGCCAAGTGTCGTACCGG |
| | | CATCTCGTGCTGCTCCACGGCGTGTGCATGGCTGGAGACAGCACCATGG |
| | | TGCAGGAATTTGTACACCTGGGGGCCATAGACATGTATCTGCGAAAACG |
| | | TGGCCACCTGGTGCCAGCCAGCTGGAAGCTGCAGGTGGTCAAACAGCT |
| | | GGCCTACGCCCTCAACTATCTGGAGGACAAAGGCCTGCCCCATGGCAAT |
| | | GTCTCTGCCCGGAAGGTGCTCCTGGCTCGGGAGGGGGCTGATGGGAGC |
| | | CCGCCCTTCATCAAGCTGAGTGACCCTGGGGTCAGCCCCGCTGTGTTAA |
| | | GCCTGGAGATGCTCACCGACAGGATCCCCTGGGTGGCCCCCGAGTGTCT |
| | | CCGGGAGGCGCAGACACTTAGCTTGGAAGCTGACAAGTGGGGCTTCGG |
| | | CGCCACGGTCTGGGAAGTGTTTAGTGGCGTCACCATGCCCATCAGTGCC |
| | | CTGGATCCTGCTAAGAAACTCCAATTTTATGAGGACCGGCAGCAGCTGC |
| | | CGGCCCCCAAGTGGACAGAGCTGGCCCTGCTGATTCAACAGTGCATGGC |
| | | CTATGAGCCGGTCCAGAGGCCCTCCTTCCGAGCCGTCATTCGTGACCTC |
| | | AATAGCCTCATCTCTTCAGACTATGAGCTCCTCTCAGACCCCACACCTG |
| | | GTGCCCTGGCACCTCGTGATGGGCTGTGGAATGGTGCCCAGCTCTATGC |
| | | CTGCCAAGACCCCACGATCTTCGAGGAGAGACACCTCAAGTACATCTCA |
| | | CAGCTGGGCAAGGGCAACTTTGGCAGCGTGGAGCTGTGCCGCTATGAC |
| | | CCGCTAGGCGACAATACAGGTGCCCTGGTGGCCGTGAAACAGCTGCAG |
| | | CACAGCGGGCCAGACCAGCAGAGGGACTTTCAGCGGGAGATTCAGATC |
| | | CTCAAAGCACTGCACAGTGATTTCATTGTCAAGTATCGTGGTGTCAGCT |
| | | ATGGCCCGGGCCGCCAGAGCCTGCGGCTGGTCATGGAGTACCTGCCCA |
| | | GCGGCTGCTTGCGCGACTTCCTGCAGCGGCACCGCGCGCGCCTCGATGC |
| | | CAGCCGCCTCCTTCTCTATTCCTCGCAGATCTGCAAGGGCATGGAGTAC |
| | | CTGGGCTCCCGCCGCTGCGTGCACCGCGACCTGGCCGCCCGAAACATCC |
| | | TCGTGGAGAGCGAGGCACACGTCAAGATCGCTGACTTCGGCCTAGCTA |
| | | AGCTGCTGCCGCTTGACAAAGACTACTACGTGGTCCGCGAGCCAGGCCA |
| | | GAGCCCCATTTTCTGGTATGCCCCCGAATCCCTCTCGGACAACATCTTCT |
| | | CTCGCCAGTCAGACGTCTGGAGCTTCGGGGTCGTCCTGTACGAGCTCTT |
| | | CACCTACTGCGACAAAAGCTGCAGCCCCTCGGCCGAGTTCCTGCGGATG |
| | | ATGGGATGTGAGCGGGATGTCCCCGCCCTCTGCCGCCTCTTGGAACTGC |
| | | TGGAGGAGGGCCAGAGGCTGCCGGCGCCTCCTGCCTGCCCTGCTGAGGT |
| | | TCACGAGCTCATGAAGCTGTGCTGGGCCCCTAGCCCACAGGACCGGCCA |
| | | TCATTCAGCGCCCTGGGCCCCCAGCTGGACATGCTGTGGAGCGGAAGCC |
| | | GGGGGTGTGAGACTCATGCCTTCACTGCTCACCCAGAGGGCAAACACC |
| | | ACTCCCTGTCCTTTTCATAG |
| 76 | JAK3 | ATGGCACCTCCAAGTGAAGAGACGCCCCTGATCCCTCAGCGTTCATGCA |
| | cDNA | GCCTCTTGTCCACGGAGGCTGGTGCCCTGCATGTGCTGCTGCCCGCTCG |
| | single | GGGCCCCGGGCCCCCCCAGCGCCTATCTTTCTCCTTTGGGGACCACTTG |
| | mutation 2 | GCTGAGGACCTGTGCGTGCAGGCTGCCAAGGCCAGCGGCATCCTGCCTG |
| | | TGTACCACTCCCTCTTTGCTCTGGCCACGGAGGACCTGTCCTGCTGGTTC |
| | | CCCCCGAGCCACATCTTCTCCGTGGAGGATGCCAGCACCCAAGTCCTGC |
| | | TGTACAGGATTCGCTTTTACTTCCCCAATTGGTTTGGGCTGGAGAAGTG |
| | | CCACCGCTTCGGGCTACGCAAGGATTTGGCCAGTGCTATCCTTGACCTG |
| | | CCAGTCCTGGAGCACCTCTTTGCCCAGCACCGCAGTGACCTGGTGAGTG |
| | | GGCGCCTCCCCGTGGGCCTCAGTCTCAAGGAGCAGGGTGAGTGTCTCAG |
| | | CCTGGCCGTGTTGGACCTGGCCCGGATGGCGCGAGAGCAGGCCCAGCG |
| | | GCCGGGAGAGCTGCTGAAGACTGTCAGCTACAAGGCCTGCCTACCCCC |
| | | AAGCCTGCGCGACCTGATCCAGGGCCTGAGCTTCGTGACGCGGAGGCG |
| | | TATTCGGAGGACGGTGCGCAGAGCCCTGCGCCGCGTGGCCGCCTGCCA |
| | | GGCAGACCGGCACTCGCTCATGGCCAAGTACATCATGGACCTGGAGCG |
| | | GCTGGATCCAGCCGGGGCCGCCGAGACCTTCCACGTGGGCCTCCCTGGG |
| | | GCCCTTGGTGGCCACGACGGGCTGGGGCTGCTCCGCGTGGCTGGTGACG |
| | | GCGGCATCGCCTGGACCCAGGGAGAACAGGAGGTCCTCCAGCCCTTCT |
| | | GCGACTTTCCAGAAATCGTAGACATTAGCATCAAGCAGGCCCCGCGCGT |
| | | TGGCCCGGCCGGAGAGCACCGCCTGGTCACTGTTACCAGGACAGACAA |
| | | CCAGATTTTAGAGGCCGAGTTCCCAGGGCTGCCCGAGGCTCTGTCGTTC |
| | | GTGGCGCTCGTGGACGGCTACTTCCGGCTGACCACGGACTCCCAGCACT |
| | | TCTTCTGCAAGGAGGTGGCACCGCCGAGGCTGCTGGAGGAAGTGGCCG |
| | | AGCAGTGCCACGGCCCCATCACTCTGGACTTTGCCATCAACAAGCTCAA |
| | | GACTGGGGGCTCACGTCCTGGCTCCTATGTTCTCCGCCGCAGCCCCCAG |
| | | GACTTTGACAGCTTCCTCCTCACTGTCTGTGTCCAGAACCCCCTTGGTCC |
| | | TGATTATAAGGGCTGCCTCATCCGGCGCAGCCCCACAGGAACCTTCCTT |
| | | CTGGTTGGCCTCAGCCGACCCCACAGCAGTCTTCGAGAGCTCCTGGCAA |
| | | CCTGCTGGGATGGGGGGCTGCACGTAGATGGGGTGGCAGTGACCCTCA |
| | | CTTCCTGCTGTATCCCCAGACCCAAAGAAAAGTCCAACCTGATCGTGGT |
| | | CCAGAGAGGTCACAGCCCACCCACATCATCCTTGGTTCAGCCCCAATCC |
| | | CAATACCAGCTGAGTCAGATGACATTTCACAAGATCCCTGCTGACAGCC |
| | | TGGAGTGGCATGAGAACCTGGGCCATGGGTCCTTCACCAAGATTTACCG |
| | | GGGCTGTCGCCATGAGGTGGTGGATGGGGAGGCCCGAAAGACAGAGGT |
| | | GCTGCTGAAGGTCATGGATGCCAAGCACAAGAACTGCATGGAGTCATT |
| | | CCTGGAAGCAG[C > T,pA572V]GAGCTTGATGAGCCAAGTGTCGTACCGG |
| | | CATCTCGTGCTGCTCCACGGCGTGTGCATGGCTGGAGACAGCACCATGG |
| | | TGCAGGAATTTGTACACCTGGGGGCCATAGACATGTATCTGCGAAAACG |
| | | TGGCCACCTGGTGCCAGCCAGCTGGAAGCTGCAGGTGGTCAAACAGCT |
| | | GGCCTACGCCCTCAACTATCTGGAGGACAAAGGCCTGCCCCATGGCAAT |
| | | GTCTCTGCCCGGAAGGTGCTCCTGGCTCGGGAGGGGGCTGATGGGAGC |
| | | CCGCCCTTCATCAAGCTGAGTGACCCTGGGGTCAGCCCCGCTGTGTTAA |
| | | GCCTGGAGATGCTCACCGACAGGATCCCCTGGGTGGCCCCCGAGTGTCT |
| | | CCGGGAGGCGCAGACACTTAGCTTGGAAGCTGACAAGTGGGGCTTCGG |
| | | CGCCACGGTCTGGGAAGTGTTTAGTGGCGTCACCATGCCCATCAGTGCC |
| | | CTGGATCCTGCTAAGAAACTCCAATTTTATGAGGACCGGCAGCAGCTGC |
| | | CGGCCCCCAAGTGGACAGAGCTGGCCCTGCTGATTCAACAGTGCATGGC |
| | | CTATGAGCCGGTCCAGAGGCCCTCCTTCCGAGCCGTCATTCGTGACCTC |
| | | AATAGCCTCATCTCTTCAGACTATGAGCTCCTCTCAGACCCCACACCTG |
| | | GTGCCCTGGCACCTCGTGATGGGCTGTGGAATGGTGCCCAGCTCTATGC |
| | | CTGCCAAGACCCCACGATCTTCGAGGAGAGACACCTCAAGTACATCTCA |
| | | CAGCTGGGCAAGGGCAACTTTGGCAGCGTGGAGCTGTGCCGCTATGAC |
| | | CCGCTAGGCGACAATACAGGTGCCCTGGTGGCCGTGAAACAGCTGCAG |
| | | CACAGCGGGCCAGACCAGCAGAGGGACTTTCAGCGGGAGATTCAGATC |
| | | CTCAAAGCACTGCACAGTGATTTCATTGTCAAGTATCGTGGTGTCAGCT |
| | | ATGGCCCGGGCCGCCAGAGCCTGCGGCTGGTCATGGAGTACCTGCCCA |
| | | GCGGCTGCTTGCGCGACTTCCTGCAGCGGCACCGCGCGCGCCTCGATGC |
| | | CAGCCGCCTCCTTCTCTATTCCTCGCAGATCTGCAAGGGCATGGAGTAC |
| | | CTGGGCTCCCGCCGCTGCGTGCACCGCGACCTGGCCGCCCGAAACATCC |
| | | TCGTGGAGAGCGAGGCACACGTCAAGATCGCTGACTTCGGCCTAGCTA |
| | | AGCTGCTGCCGCTTGACAAAGACTACTACGTGGTCCGCGAGCCAGGCCA |
| | | GAGCCCCATTTTCTGGTATGCCCCCGAATCCCTCTCGGACAACATCTTCT |
| | | CTCGCCAGTCAGACGTCTGGAGCTTCGGGGTCGTCCTGTACGAGCTCTT |
| | | CACCTACTGCGACAAAAGCTGCAGCCCCTCGGCCGAGTTCCTGCGGATG |
| | | ATGGGATGTGAGCGGGATGTCCCCGCCCTCTGCCGCCTCTTGGAACTGC |
| | | TGGAGGAGGGCCAGAGGCTGCCGGCGCCTCCTGCCTGCCCTGCTGAGGT |
| | | TCACGAGCTCATGAAGCTGTGCTGGGCCCCTAGCCCACAGGACCGGCCA |
| | | TCATTCAGCGCCCTGGGCCCCCAGCTGGACATGCTGTGGAGCGGAAGCC |
| | | GGGGGTGTGAGACTCATGCCTTCACTGCTCACCCAGAGGGCAAACACC |
| | | ACTCCCTGTCCTTTTCATAG |
| 77 | JAK3 | ATGGCACCTCCAAGTGAAGAGACGCCCCTGATCCCTCAGCGTTCATGCA |
| | cDNA | GCCTCTTGTCCACGGAGGCTGGTGCCCTGCATGTGCTGCTGCCCGCTCG |
| | double | GGGCCCCGGGCCCCCCCAGCGCCTATCTTTCTCCTTTGGGGACCACTTG |
| | mutation 2 | GCTGAGGACCTGTGCGTGCAGGCTGCCAAGGCCAGCGGCATCCTGCCTG |
| | | TGTACCACTCCCTCTTTGCTCTGGCCACGGAGGACCTGTCCTGCTGGTTC |
| | | CCCCCGAGCCACATCTTCTCCGTGGAGGATGCCAGCACCCAAGTCCTGC |
| | | TGTACAGGATTCGCTTTTACTTCCCCAATTGGTTTGGGCTGGAGAAGTG |
| | | CCACCGCTTCGGGCTACGCAAGGATTTGGCCAGTGCTATCCTTGACCTG |
| | | CCAGTCCTGGAGCACCTCTTTGCCCAGCACCGCAGTGACCTGGTGAGTG |
| | | GGCGCCTCCCCGTGGGCCTCAGTCTCAAGGAGCAGGGTGAGTGTCTCAG |
| | | CCTGGCCGTGTTGGACCTGGCCCGGATGGCGCGAGAGCAGGCCCAGCG |
| | | GCCGGGAGAGCTGCTGAAGACTGTCAGCTACAAGGCCTGCCTACCCCC |
| | | AAGCCTGCGCGACCTGATCCAGGGCCTGAGCTTCGTGACGCGGAGGCG |
| | | TATTCGGAGGACGGTGCGCAGAGCCCTGCGCCGCGTGGCCGCCTGCCA |
| | | GGCAGACCGGCACTCGCTCATGGCCAAGTACATCATGGACCTGGAGCG |
| | | GCTGGATCCAGCCGGGGCCGCCGAGACCTTCCACGTGGGCCTCCCTGGG |
| | | GCCCTTGGTGGCCACGACGGGCTGGGGCTGCTCCGCGTGGCTGGTGACG |
| | | GCGGCATCGCCTGGACCCAGGGAGAACAGGAGGTCCTCCAGCCCTTCT |
| | | GCGACTTTCCAGAAATCGTAGACATTAGCATCAAGCAGGCCCCGCGCGT |
| | | TGGCCCGGCCGGAGAGCACCGCCTGGTCACTGTTACCAGGACAGACAA |
| | | CCAGATTTTAGAGGCCGAGTTCCCAGGGCTGCCCGAGGCTCTGTCGTTC |
| | | GTGGCGCTCGTGGACGGCTACTTCCGGCTGACCACGGACTCCCAGCACT |
| | | TCTTCTGCAAGGAGGTGGCACCGCCGAGGCTGCTGGAGGAAGTGGCCG |
| | | AGCAGTGCCACGGCCCCATCACTCTGGACTTTGCCATCAACAAGCTCAA |
| | | GACTGGGGGCTCACGTCCTGGCTCCTATGTTCTCCGCCGCAGCCCCCAG |
| | | GACTTTGACAGCTTCCTCCTCACTGTCTGTGTCCAGAACCCCCTTGGTCC |
| | | TGATTATAAGGGCTGCCTCATCCGGCGCAGCCCCACAGGAACCTTCCTT |
| | | CTGGTTGGCCTCAGCCGACCCCACAGCAGTCTTCGAGAGCTCCTGGCAA |
| | | CCTGCTGGGATGGGGGGCTGCACGTAGATGGGGTGGCAGTGACCCTCA |
| | | CTTCCTGCTGTATCCCCAGACCCAAAGAAAAGTCCAACCTGATCGTGGT |
| | | CCAGAGAGGTCACAGCCCACCCACATCATCCTTGGTTCAGCCCCAATCC |
| | | CAATACCAGCTGAGTCAGATGACATTTCACAAGATCCCTGCTGACAGCC |
| | | TGGAGTGGCATGAGAACCTGGGCCATGGGTCCTTCACCAAGATTTACCG |
| | | GGGCTGTCGCCATGAGGTGGTGGATGGGGAGGCCCGAAAGACAGAGGT |
| | | GCTGCTGAAGGTCATGGATGCCAAGCACAAGAACTGCATGGAGTCATT |
| | | CCTGGAAG[C > T,p.A573V]AG[C > T,p.A572V]GAGCTTGATGAGCCAAGTGT |
| | | CGTACCGGCATCTCGTGCTGCTCCACGGCGTGTGCATGGCTGGAGACAG |
| | | CACCATGGTGCAGGAATTTGTACACCTGGGGGCCATAGACATGTATCTG |
| | | CGAAAACGTGGCCACCTGGTGCCAGCCAGCTGGAAGCTGCAGGTGGTC |
| | | AAACAGCTGGCCTACGCCCTCAACTATCTGGAGGACAAAGGCCTGCCCC |
| | | ATGGCAATGTCTCTGCCCGGAAGGTGCTCCTGGCTCGGGAGGGGGCTGA |
| | | TGGGAGCCCGCCCTTCATCAAGCTGAGTGACCCTGGGGTCAGCCCCGCT |
| | | GTGTTAAGCCTGGAGATGCTCACCGACAGGATCCCCTGGGTGGCCCCCG |
| | | AGTGTCTCCGGGAGGCGCAGACACTTAGCTTGGAAGCTGACAAGTGGG |
| | | GCTTCGGCGCCACGGTCTGGGAAGTGTTTAGTGGCGTCACCATGCCCAT |
| | | CAGTGCCCTGGATCCTGCTAAGAAACTCCAATTTTATGAGGACCGGCAG |
| | | CAGCTGCCGGCCCCCAAGTGGACAGAGCTGGCCCTGCTGATTCAACAGT |
| | | GCATGGCCTATGAGCCGGTCCAGAGGCCCTCCTTCCGAGCCGTCATTCG |
| | | TGACCTCAATAGCCTCATCTCTTCAGACTATGAGCTCCTCTCAGACCCCA |
| | | CACCTGGTGCCCTGGCACCTCGTGATGGGCTGTGGAATGGTGCCCAGCT |
| | | CTATGCCTGCCAAGACCCCACGATCTTCGAGGAGAGACACCTCAAGTAC |
| | | ATCTCACAGCTGGGCAAGGGCAACTTTGGCAGCGTGGAGCTGTGCCGCT |
| | | ATGACCCGCTAGGCGACAATACAGGTGCCCTGGTGGCCGTGAAACAGC |
| | | TGCAGCACAGCGGGCCAGACCAGCAGAGGGACTTTCAGCGGGAGATTC |
| | | AGATCCTCAAAGCACTGCACAGTGATTTCATTGTCAAGTATCGTGGTGT |
| | | CAGCTATGGCCCGGGCCGCCAGAGCCTGCGGCTGGTCATGGAGTACCTG |
| | | CCCAGCGGCTGCTTGCGCGACTTCCTGCAGCGGCACCGCGCGCGCCTCG |
| | | ATGCCAGCCGCCTCCTTCTCTATTCCTCGCAGATCTGCAAGGGCATGGA |
| | | GTACCTGGGCTCCCGCCGCTGCGTGCACCGCGACCTGGCCGCCCGAAAC |
| | | ATCCTCGTGGAGAGCGAGGCACACGTCAAGATCGCTGACTTCGGCCTAG |
| | | CTAAGCTGCTGCCGCTTGACAAAGACTACTACGTGGTCCGCGAGCCAGG |
| | | CCAGAGCCCCATTTTCTGGTATGCCCCCGAATCCCTCTCGGACAACATC |
| | | TTCTCTCGCCAGTCAGACGTCTGGAGCTTCGGGGTCGTCCTGTACGAGC |
| | | TCTTCACCTACTGCGACAAAAGCTGCAGCCCCTCGGCCGAGTTCCTGCG |
| | | GATGATGGGATGTGAGCGGGATGTCCCCGCCCTCTGCCGCCTCTTGGAA |
| | | CTGCTGGAGGAGGGCCAGAGGCTGCCGGCGCCTCCTGCCTGCCCTGCTG |
| | | AGGTTCACGAGCTCATGAAGCTGTGCTGGGCCCCTAGCCCACAGGACCG |
| | | GCCATCATTCAGCGCCCTGGGCCCCCAGCTGGACATGCTGTGGAGCGGA |
| | | AGCCGGGGGTGTGAGACTCATGCCTTCACTGCTCACCCAGAGGGCAAA |
| | | CACCACTCCCTGTCCTTTTCATAG |
| 78 | PD-Li | GGCGCAACGCTGAGCAGCTGGCGCGTCCCGCGCGGCCCCAGTTCTGCGCA |
| | gDNA full | GCTTCCCGAGGCTCCGCACCAGCCGCGCTTCTGTCCGCCTGCAGGTAGGG |
| | length | AGCGTTGTTCCTCCGCGGGTGCCCACGGCCCAGTATCTCTGGCTAGCTCG |
| | | CTGGGCACTTTAGGACGGAGGGTCTCTACACCCTTTCTTTGGGATGGAGA |
| | | GAGGAGAAGGGAAAGGGAACGCGATGGTCTAGGGGGCAGTAGAGCCAATT |
| | | ACCTGTTGGGGTTAATAAGAACAGGCAATGCATCTGGCCTTCCTCCAGGC |
| | | GCGATTCAGTTTTGCTCTAAAAATAATTTATACCTCTAAAAATAAATAAG |
| | | ATAGGTAGTATAGGATAGGTAGTCATTCTTATGCGACTGTGTGTTCAGAA |
| | | TATAGCTCTGATGCTAGGCTGGAGGTCTGGACACGGGTCCAAGTCCACCG |
| | | CCAGCTGCTTGCTAGTAACATGACTTGTGTAAGTTATCCCAGCTGCAGCA |
| | | TCTAAGTAAGTCTCTTCCTGCGCTAAGCAGGTCCAGGATCCCTGAACGGA |
| | | ATTTATTTGCTCTGTCCATTCTGAGAACCCAAAGGAGTCCTAAAAGAGGA |
| | | ATGGAGGAGCCTAAGAATAAAAATAGTATAATAAAACATTTCTTAGACAC |
| | | ATTGACCTTGGCCTATGTCAAAGTTCAGTCTGGGTTTGTCTTATAACACA |
| | | AGGAGTAAAAGTACCATTGTTCTACCTCTTTTTTTAATACTTGAAAAAAA |
| | | TTTACTGTGGATGCTTTTCTATGAATTAAATAACCTTCTAAAAAATGTTT |
| | | TCATTGCTGCATTCGATTAGATTGGGTAACTAAATGAAATTAATTCCTCA |
| | | CTGTTGGGTATAAAGGTTATTTACAGTGGTTCTGTCTTAGCCATTCACTG |
| | | AACTCATTGCATATATATCTCTGGAATATTGCTGATTGTTTCCTTCAAGT |
| | | AAACTTAGAAGTGTAACTACTTAGTCAAAGAGCCTGAATATTTTAAAGGC |
| | | CTTTTGAAGAAAACTGAAAATGCTTTCCAGAAAGGATGTATCAGTTGACA |
| | | ATGACAGTCGTCAACAGTATTTAAGGAGAACTATGATACTCTGAAGAAAA |
| | | ACTTAGCCTTTCTCAGTAAAAGTAGGTAGGCAGAGGCCACATGACAGCAG |
| | | TTAGAGTGTGGTCTTCAAGGAAGTCACAGAAATACTGTGGGGAATTGAAA |
| | | CCCCATGTGGAAAATGTACAAGAGTGTCTCAGTGTGACTGAGAAGGAGGT |
| | | TGGGCATGGGGTTTCATGGAGTTTAATAAAGTTTGGTCACTTAGTAGAGG |
| | | TTTAATAAATCAACTGTCTTAATCTTTGATCCTACTTAAGAATTTTTTTT |
| | | TTGTTTTTGTAGAGATGGGGCTCTTGTTATGTTGCCCAGGCTGTTCTCGA |
| | | ACTCCTAGCCTCAGGCGATCCTCCCTCCTCAGGCTCCAGAAGTCCTGGGA |
| | | TTACTGGCGGGAGCCACCATGCAGGCCTCTTGCTCCTACTTTTGAGAAAG |
| | | GAAGTTTAACCGGTTTTTTTTGTCTTTTTTTTTTTTTTTTTGAGACAGAG |
| | | TCTCACTCTGTTGCCCATGCTGGAGTGCAGTGGTGCAATCTCAGCTCACT |
| | | GCCTCCCGGGTTCAAGTGATTCTCCTGCCTCAGCCTCCCGAGTAGCTGGG |
| | | ACTACAGGCACCTGCCACCACGCCCAGCTAATTTTTGTATTTTTAGTAGA |
| | | AATGGGGTTTCACCATATTGGCCAGGCTGATCTCGAACTCCTGACCTCAG |
| | | GTGATCCGCCTGCCTCGGCCTCCCAAAGTGCTGGGATTACAGGCATGAGC |
| | | CACTGCTCCTGGCTGCTTAACTTTTTCTCTATCTCATCCTCCTACCCATC |
| | | CTACCCTTGGAAGATAGAGAAGTAGTATTAGTTCCATAGTGTTATACTGG |
| | | GCTTCCCCCAGGGACAAACCCACTTCCCCAACCTGAATGAGCCATCACTT |
| | | CTTCCCCAGTTTACATTTCATTGCTCTTTAAATGTCTCCATTCGGATATG |
| | | GGAATTCACATATGGTCATAATTCTTACCTGAAGAAGATGTCAGTCTTCT |
| | | TCTCTTAGACCAACTGCCCTGATATGAGGTTTAGAGGTTAAAGAACATGT |
| | | GTGTATTTACATGATCTTTGTATTCTGCCTTTTCGTCCCTCACTAATGAC |
| | | AGCTGCACCCCAAGGAAATGGAGCTGTGGAAGAGAGGGTTTGATAAGAAA |
| | | TTAAGTAAATATTGGATCTAATCCATCACCCTCCAGGAAGCCTTTATTAC |
| | | TCCTAAAAATTTCAACCAAATTCATTAAAGGACAAGAACTCCACCAGAGT |
| | | AGGCCATAAACATTGGCAAAATTAGTTGTAATCCATGACTAGATTTAATG |
| | | TCCCTTTGTTTTATTCCCATATGGTTATAATGCTTTGCTTGGCATTAGGG |
| | | GTATTTTAAGTTTTCTTCTGCCTAGTAAGTGAATTTGTGTTTATAATACA |
| | | ATAATCATAAAATATCACATTAATATTTTATAACTGTACAGTTATAAAAT |
| | | ATTTTATAAGTAATATTTATATTTTATAAGTAATATTTTATAACTGTACA |
| | | GTTAACTCTGGCCCAAGGAAAAGATAGTCTGATAGATGCTGCAGCCCCAT |
| | | TTTAGCAAATGTGACCTCACAGGCCTGAATGCCATCGCTATTCCACATCT |
| | | ACAGGATAGACGGAAAGGAAAGAAATAAAAAAATAGGTACCTAACACTGG |
| | | CAAGAGGATGATGACTCATGTTATTTCACTTAACCTTTTTATCTTTTAAC |
| | | ATGAAGGACTCATACAGGTTGATAAGAAACCAGTGACATAAACAGACCAA |
| | | AAAATGATCAGATCTTTCAAATTAGCAAAAAAATAATATTTTTTAAACAA |
| | | TGGGTGAAAATACAGTGTAACAGTACCAATTATCAACATGTGTTGAGAAC |
| | | CAGAAAAATGTTCTTTTTCTTTGATCAGCAACACTATTTGGGAAAATCTA |
| | | TCCTCAGGGCCTAGCCTGGGGCCCTGGCACACAGTAGGCACTCAACGAAT |
| | | ATTTGCTGAACACACAAATACTTATGATATTTTAAAAAATTGGCAACAAT |
| | | CTGATACCTAACAATAGAGGGATTAAATATTATGGAACTGTTAAATAAGA |
| | | TGCTTATGAATACCATGCAGTAAGATGGGCAATATTTATGCCATAAGCTT |
| | | TAATGAAACAAATGGGTATTAAATGTATGATAAGGTTATAAATTACTTTT |
| | | TAAAAGATTACAGGGAAAAAAATTGAAAGATATACACTGAAATGTTTTTT |
| | | GCTCACAGTGGTGACAAGGTTTCTCAGCACTGGCACTGTTGACGTTTTAG |
| | | GCTGTATGTCTTTGCTGTGGGAGGCTGGCCTGTGCACTGCAGGGTGTTTG |
| | | GCAGCACTCTTGGCCTCTGCCCCTAGATAGCAATAGCAGTCCTCCCTCAA |
| | | CCAGCCCAATTTTGACAACCAAAAATGTTTCCAGGCATCACCAGATGCTC |
| | | CCTGGGTGAGAGTGATGAAATAGTAGGGGATTTTCCCCTTCTTTTCTTAT |
| | | TTTCTGTAATTCCATTATATTACTTTAATAATAAAGAAAAAAACATAAAA |
| | | AATAAACGAATGTTATTATTCTACGTCAGTTTGGATGTTTGGACTCCATT |
| | | TTGGGGTTCTTTCCATTATATCACTTGGTCTGCTAAACATTCTACGGTTT |
| | | GGTAAGGTGAAGTGATTCATGAAATTTTGGTTTTATTTTTTTCCTGATAC |
| | | TAAAAATAAAACATTCTTTCACTTGGAAATTTGGACACAGAACACCAAAA |
| | | AAAATCCATAATCTCATCTCTCTTTTTCTGTCTTTTCCTTCCTTTTTTCC |
| | | CTTTAAAAACAATAAAGAGTGAAACCTACCTGTTCTCCCTCTAATTTAAT |
| | | TCCTAAATATAATCACTGTCAATATCTTGGACATTTCCTGTGTCTAAACA |
| | | CACACACACACTTTTTTTTTTCAGCAAAAGTGGATTTCTGCTACATGTAG |
| | | TGTTCTGCAACTTACTTTCTATGTGTTTACAAAATCAGTACATGTACATA |
| | | TGCTGAATTCAGTCCTTAATGGTATTATATTTTGTGAATATACCAAAATT |
| | | TGTTTAACCACTTAGACAATCTAGGATATTCTCAGTTTGCTGTTATGAGC |
| | | AATGCTCTTCCTTTACATATACAGACATATATATATATATGTGTGTGTGT |
| | | GTGTTTTTGTTTTAGTAGGATAGATTTCTAGGAGAGGGTGAAAGGTCTTA |
| | | TGACATCCGCATTTACGATTGTAATAGGAAGTATCAAAGTGCCCCCTAAA |
| | | GAAAAAAATCCTCCCATTAGTGGGTAAGAAAGCCTATTTGTTCATATCTT |
| | | CACAAACACTAAATATTAGAAATATTTACAATTGTGGTCAAGCTCATAAG |
| | | TGAAAATGGTATTTCATATCTTATATTTTTTATTGTGAGATTGAACATCT |
| | | TTCATATGTTTACATGTCACCTGTATTTCTTATTCTCTGAACTATATGTT |
| | | ATGACCTTTCACTTTTTTTCCTCATGGGTTATGTGTAGTTTGTATAGTTG |
| | | TCTTATTGATTGTTAGGAGCTATTTATATATTAGGAACATTAATCTCCTG |
| | | TCTTATATATACGTGGCATCGATTAGTTGATCATTTGTGAGTTCATGTCT |
| | | GTATACAAAGATTGGAGAGGCACTAAGAGGGAAAACTTACCTCTTTCTTA |
| | | TCAAAGTTTGTAAATATATGTATAACAGAAGAGGGAGAAAATATTAATAA |
| | | ATGCACAGATTGGCTGAAATAGAGTATAAATCTTTTACTCCCCTACTTCA |
| | | ACATAAACTGCAAAAGGAGAGTGACTTTTCTTTCACTCTGACTTCCGTAT |
| | | TCCTCATGCTTAAAATAGTGCCTAGCACAGAAGAGGTGCTCAATCAGTGT |
| | | TTGCTAAACGAAATAATTAGTCACATTTCAAGCAGGATGACTAAATGAAG |
| | | AATAGAATCTAGGCAGATACTCTGGAAGAGTGGCTGTGAGTCATTCATAT |
| | | CTTAGTATGAATTAGTCAAATCCAACTCTCTCCCCTTCCCACTCCCCACT |
| | | GTTAGTAGAAGAATCTGTTTATTGAGAGAATAGATTTATAATTTAGAATA |
| | | AGTGAGAGGGGCAGAAGAGGAGATTTTGAAGGATGGCACCTGAAGGAGGA |
| | | CTAGCATGGCTGAGACAGTGAAGTGGAAGCCTTGAATAGCTAAAGGGTAA |
| | | GATGAAAGTATTTAGCTGTAGGGGGAAAAAGCATTGACAGGTTGGAAAAG |
| | | TAAAAGTCAGATTCTCCTTGCTCTGAAATTTTGTACAGGGCAGGTTCTAC |
| | | TAGGTATGTTACAATGCAGAAAAAACATGAAATAATTGAGAGGAATTTGG |
| | | TGCAATATTATCTTCTTGGCTTCTTTTGAGTGGGCAGATTTTTTTCACGG |
| | | CCTGTAACTATAATAAATTTGAAACTTCTCATCTTTTAGTAACTTTTTTC |
| | | ACTTAAGTTTATGTGGCTGTGGGCAATGGAATGAAGATATTGAACTTCCA |
| | | ATTCCCTGTTGGGTTTCCACAATTACAAGTCAATCATGACTGGTTATTAG |
| | | AAGACTATTTCAGTTAGAACCACCAAGTCCCATATTGTCATATTGTATGT |
| | | TTAATTATTAAGTGAAGCAGTCTTCTTTTCGTGTTTTCCATAATTAGGGC |
| | | ATTCCAGAAAGATGAGGATATTTGCTGTCTTTATATTCATGACCTACTGG |
| | | CATTTGCTGAACGGTAAGACACCAAATCCTTCCATTAGGTTCTATATTTT |
| | | AAATATTTTAACCATGAGTTTAAAACTAAAATGATCATTTAAAATGCATG |
| | | CAATTTTCTTATAGAGAGAACATTCTATTCTTTCTTCTACTTTACACAAT |
| | | GGCAAAGTCTTCTTTCTACTTTACGCAATGATAAAGTTACCTGTGTCATT |
| | | TTGTAAAAATATAGAGAATATAGACAAATTGAAAGACACAAAATAATCTA |
| | | TTACCCATTTCCCAGGGTTAACTACTGAAAATATCTGGGGAAATGGCCTG |
| | | TATGTATACATTTATTTGTTTGCTTTCAACAAGGCCAAGATCCTTTGATC |
| | | TTTCAGTCTTGGTTGCTCTGTGACATGCCTTTCCTGATGAGGATACTTTA |
| | | AGGAAGAATTGTAAGATACATGGAAAATGTCAGGCTAACACAGTACTGGC |
| | | ATCACCCTGTGCTCTTTCCTGAACTCCATACCAATGTACTTCTTGCCAGA |
| | | AAACTGATCAAAAGTTTAGGGAAGTAAAAAGAGATGACTGTTAGAATCTA |
| | | CCATTCCCTCTATGTAGGAAGCAAATAGGTGTCCTGTCAAAGGACATTCT |
| | | GGGGATGTCTACATGAAACCAACTCTCCCTGGTTGTAAGGACTCCATCTC |
| | | CATATAATATTTATACAGTAATATATGTTTATAAATTGTGGGGGCAACTT |
| | | GTTTAGCTAATTTTATTATTCTGCTATTGGGACACTGTGTCTCAGCATGA |
| | | GATATAGTGTCCCAAAACATATTTCAAGCCCATTGGATAAAATATGTGTT |
| | | TAGCAAGTTCTTAAATATAATGATAACATAACCGACCAGATAAAGTGATT |
| | | TATAAACGCTGTGCCAATTTTGTAAATGTTTCGAGGAATTTTCCCTTTTC |
| | | TGAAGATTGTCCTTCTTTCTTTTTAGCATTTACTGTCACGGTTCCCAAGG |
| | | ACCTATATGTGGTAGAGTATGGTAGCAATATGACAATTGAATGCAAATTC |
| | | CCAGTAGAAAAACAATTAGACCTGGCTGCACTAATTGTCTATTGGGAAAT |
| | | GGAGGATAAGAACATTATTCAATTTGTGCATGGAGAGGAAGACCTGAAGG |
| | | TTCAGCATAGTAGCTACAGACAGAGGGCCCGGCTGTTGAAGGACCAGCTC |
| | | TCCCTGGGAAATGCTGCACTTCAGATCACAGATGTGAAATTGCAGGATGC |
| | | AGGGGTGTACCGCTGCATGATCAGCTATGGTGGTGCCGACTACAAGCGAA |
| | | TTACTGTGAAAGTCAATGGTAAGAATTATTATAGATGAGAGGCCTGATCT |
| | | TTATTGAAAACATATTCCAAGTGTTGAAGACTTTTCATTCTTGTAAGTCC |
| | | ATACTTATTTTCAAACAGAACAGCATAGTCTGTTCATTCATTCATTCAAT |
| | | TCATGAATTCATTCACATAATTATCCAATTTCTTGAGCACCTATTTGATA |
| | | GTCACTGGAAATCCAGAGACAAACAACACAGAGCCATGTTCTACAGTATG |
| | | TACAGTTTTCCAAAAAGAATTTCTAGTCTTTACTTTTTTATTACAAATGG |
| | | AATACGTATACTTGCAAATAATTCAGATACTGTGGAAGAGATCAAATGAA |
| | | TTGCAAAAGTGTCCCTCCTCCCTTCACCACTATCTCCCATGGCATGCAGA |
| | | GAGAGTAACCATTATTTGTGTGTCCCTCCAGAAATTTTTTTATTCAACTA |
| | | CTATTTTTTTATTTTATTAGGTCCGTCAGTTTTCCTTTTTTGAGCCTCTC |
| | | TATATCAAATGCAAATAAATATATTCAGAACAAACCCCACTGTAAGGTTC |
| | | ACATTAAAAAAGACTTGAAGTCACCCTATGAAGACAAAAAATAATCACAT |
| | | TAAGTGTGAAAGAACCTATTCTTCCAGTACAGGATAAGCCATACTTACTG |
| | | GGCATATATTCATCTTGAAAATCTATACTGATGTTGTCTTGGGGAATTGA |
| | | AAAGGAACTAGGAGTGTTAGTTCCTCGGTATTGACCCACAGTTATGTTAT |
| | | CAGGTCACTTGAGTTCAAAGTTTTGTGTTGGCACTAGCTAAGTAAAGGAA |
| | | AACACCTCTGCTTTCATTGTTGAGTTTCACAGAATTGAGAGCTGAAAGGA |
| | | TCCCAGGCAGGAGCAGCTAATCCAAACTCCCACAAAGAACAAAAATCCCC |
| | | CAGAGGATCTTCTGTTCTTATATTTCCTGCAATGGCGTCCCTGTCATATC |
| | | CCACAATGGCCTCCCTGCCATTTGGATATCCCTTCCATATCCTGTTGAAA |
| | | TTACTCCCTAATAGTAAGCTGAAATCTGCCCCTCTAGTTGTAGTCTTGGG |
| | | ATTATTTCATTTACATGATGACCTTTTAATATTTGACTAGAATTAAATCA |
| | | TCTCCCCTTGGTCTTTCCATTCCTGGGCTAACTACCATCAATCTGAGGGC |
| | | TAACAATACAAGTAGAAAAAGTATACATTTGTCACTGATCACTGATCAAT |
| | | TATTAATCAATGATCACTGATAACTATAAACTCAAAAACAAAATCATGTG |
| | | GGGATTAAGAGAAATGTATCAGTTTTATGTTGTATTTCTGGTCCCTGATA |
| | | CTGGCTCAGGTAATGCCACTATTGTCAAGAAGATACCACTTGTAAAGTAG |
| | | ATTTAATTTTCATTATATTTTACCATATGCTTCTCCATTCATGACATCTC |
| | | TTGAGATGTTGTGGTTTATACTTTCAGTTTTTCTCCAGTCCATCCGCAAA |
| | | TATCAGGCATCTACTGTGTTCCAAGATATTAAAGAAATCATCATGACTTA |
| | | GCCTCATCAACAGCATTGCTAGATCTGGGATGGAAAGGAAGAGTATAATC |
| | | CTGGCAGTCAGGAAGAAGGCAGCATAAAGTATAAGTTTCTGCTTCCAAAA |
| | | AAGGTCTCTCATCAGCCTGTAGGGAGTGTGTAGGGAAGGGACAGCTGTCC |
| | | TTGTAGTAGGGAAGGGTTTTATTCAGGTCGTCTGGGCTCCATAATATCCC |
| | | TTGTGTATCTGCAGTCTCCTTTGCCATGGATCAACACAATAGGAAATCTT |
| | | CCGGCACTGATGGTTTTTCCAAGGGGGAGTTCTTCCTGGAGCAAAGCAAA |
| | | TGACCAACCAGGTTTGAGGACCTGATTTGTTTGACAATTCCATTTTGTAT |
| | | TGTAAATTACTTAATTGGCATTCTACTCCCAATCCATCTTGTCATTTGCA |
| | | TACAGTGGTTTTGGGATTGAGTTCAGCTATACCAAAAGTCTGAACCTTCT |
| | | GCACTTAGAACAAGGCAACCACCAAGCTTCACTTGCACTGAGGCCGTGTC |
| | | TCCAATGGAAATGAGGCAGCTGGCTTGCAGGAGCTTCCCAACTCAGGGAA |
| | | GTAGAACTCCTGAGTCACCTCCATATGCAAATGATTTCACAGTAATGCTG |
| | | TTGAACTTCACTTCCCATCACAGCAAATGTGTGGTAACATAGCTTCCCCA |
| | | CAGGAGTTTACTCACCATGGTATTTTAAAGGTGAAACATTTCAAAACTGA |
| | | AATTTGAAAGAATTTAGTTTTGGATTCACTCAATTATCACTATCACTTCG |
| | | GGTGTTATTGCACCTTTCTTGTTTGTGAGTTTAAATGCCAGACTCTCAGG |
| | | CCACTAACTTTCAATTAAAAGTGTTTTTCTTTAATCGCTGAACCTAACAG |
| | | CAGGGAAAACGAAATGTTCATTCAGACTTTCAGAACCTTCAATGAGATTA |
| | | GGCAGCTGAAAGATCAAAGTGTTGCATAGTTGTCCCGATAAAGCTATTTG |
| | | GATCATATGGACCAAATCGACTGCTGTCATTCCCCACCAACCCCATCTCT |
| | | CCCCAAAATTCCCAGCCCTGTTTAAGTGTTCTCTGTAGCATTTATCTCTA |
| | | TCTAGTATATTGTGTAGCATATCATATCATACTTTTCTGTTTTGTTTATT |
| | | GTCTCTCTCCTCCTAGAATATAAACTCCACAAGCACAAAGATTTGGGCCT |
| | | GTTTTATAATATTGTTGCATCCCCAGGGCCTGATATACAGCAGAGTGGTG |
| | | GTACGAAAAGAGCACACAAAAAAATATTTGTTGAGTCAATGAATGAATGA |
| | | TTTCCTCAAATAGGATTAGCCTAAAATTTTGGAAACATGAACAGATTTGG |
| | | ATATGTGAAAATTTATTTCCAGACTGTTCATCAGGAACTGTTAGCAGCTT |
| | | CTAAAGGGTACACTGGAGCAGCAGTAGTAAAAGGAGGAAGAGGAGCAGCT |
| | | CTGCTACTGCTACTATCGAGTACTACTACAATTAGCACTTGCTTATTCTG |
| | | TGTGTTAGGCCCTGTACTGAACACTCTGTCTAAATTAGTTCATTTCCTCC |
| | | TGGAAATGACTCTAGGGGGTAAGTGCTTCATCATGTAAGATGAGTATTTT |
| | | TCACATTTTGTTGTGTCTGAAATCTGAGTGTGTCTTTCAATGATGGAATC |
| | | TTTGATTCCATGATAAGTGGTATTATTCCCATTTTAAGGATGAGGAAACT |
| | | GAGGTCCAAAGAAATTAAGTAATTTGCCCAAATTCACCCAGCCTAGAAAA |
| | | TGATAAAGCTAGTTCTAAACCCAAGCAGATTAGCTCTGAAGTCTGGGCCC |
| | | TTAATAACCACTTTTTATTGCCTATATTTGTACCTCTGGTGTACGTATCA |
| | | AGTTATATGTTGACTTCAAAACTATCATGACCTTTTCTTGGTTTTGATTG |
| | | TCCAACATTAGTATAGTGTTCTGGGTCTGCAAAAATTTTGATTACTCATC |
| | | TCATCTGTAAAACATTTTGAACTCGTGTGTTTGTGCATGCACATTTGTGT |
| | | GTAATTATAAAAATTTTACTTTCTGTTAATATATAAGTTGTATCATAAGA |
| | | AACTGCCGTTTTTGAAGAGCAAAAAAAGGTTGAATGTTACCAGTTACATC |
| | | TGGTTCAACCTAATAGACATTTGTACAAAAACAGACATTTTAAGAGGTTG |
| | | AAATAAAAATTTAATAAACAATATTTTCAGTTTTTACTAATTGTGATGCT |
| | | TCACTATCATTAGCTAATATGTCAAGGCATAATATACCTTAGGGTGAACT |
| | | TTATCATTAACAAAGGTGGATGGTGTCAATAATCTTGAGGTTTGTGTTTT |
| | | TTTATATAACACTGCGAGGTCTAATTAAGTACTTACTGTTTACCACCTCA |
| | | TACAGTGGCCGATAAAAAGTGTCACTTCTGCTGTTTCCTCTGGGTTGTGC |
| | | TTGAATTATTAGTATTATCTTCAGTCCTCAGTTTCTTTGTGGGAAACTTT |
| | | TTAATTAGTTGTTTAATTTTGTAAGATGGTTAGTTTAGTCAAAATTAGAT |
| | | AAGAGAATTTGAAAATCCGTAGCTACCCCAAAGCAACCTACACATAAGAA |
| | | CTATTATTTTTGTGTTTTGAAATCATAATTTTATTGATTTCCAGTGTTTC |
| | | CACTGGTAGTGGTTTCATTGATATAGGAGTATCAAAACATCACTCATTAT |
| | | TTATTTCAGTTTCATTTGATCCTAGCCGTTTTGTATTAACTCTCTGTGAA |
| | | GAAATTACCTCACAAATCTATTGCTGTCCTTGGTAAAGGAATGGAGAATT |
| | | AAGGCTCTAGATCATTAGTGGTTACACTATAGTATTAGAAGTAAAAAAAA |
| | | GATTATACCAACAAAATAAGAACATGTTAATGTACTTGTAATGAATAAAC |
| | | ATGAATAAAGCTCTTATGCTATATAGGTGCACTAAACAATCTACTAGAAT |
| | | TGTCAGCAAACTACGTATCTTAATCCTGAAAGGGTCCCAAACCAATGATC |
| | | TAAAATTGAATCAAACTTTCTTCCTTGAGCATAATTACTTAAATGATTTA |
| | | TTAAAATAGCCAGCATTTAAAAGCTTAAAATGTAAATATCATAATGTGGT |
| | | ATCCTAGATAGCATCCCAGAACAGAAAAAGGATATTAGGGAAAAACTGGA |
| | | GGAATGGAATAAATTATGCAGTTTAGTTATTAATAATGTACTAACGTCCT |
| | | TAGTTATGACGATTGTACCATGGTAATGTAAGATACTAACAATAGAGGAA |
| | | ACCGGGTAAGGAGTATACAGTAACTCTATACTATCTTTGCAACTTTTTTG |
| | | TAAATTTAAAACTTCTAAAATAAAGAACAAATTTAAACATTAAAAAGTAT |
| | | CACCAGGAACATATATCACTGTTTACAGATGAAATACTATGTATTTTCAT |
| | | ATCTAATTTCTGATCATTGACTTCAAATCAGAAAAGTGAATGACACCTCA |
| | | AAATCAGGTTTTCTGTTTACTGAAGTCTAAGAAAAGAAAGCATACCAGCT |
| | | GGAGAGATTCATGTTTATAAAGACAGATTTATAACAACAAAAATAAAATA |
| | | TCCAAGAATAAATTTAAGAAGAAGCACTTTACTGAGAAACATATGAAAAC |
| | | CTGAACAAATGGAGAGGGATATTTTGTATTTGAATAGAAAGACTTCTGGT |
| | | TTAAAGATAATTCTCTTTAAATTATTTTTTGTAGAAATTTAAGGGGTACA |
| | | AGAGCAGTGTTGTCACATGGATATATTACATAGTGGTGAAGTCTGGGGTT |
| | | TTAGTGTAAATTAATCTTTACATTTTGTTTGAGCCCAATAAATGTACCAA |
| | | CATGATTTTTATAGAAAGATAGTCATTCCTATTAATCCAAACTTGTCCCA |
| | | ACTTTGAATTGAATTGAGGCAGAGCTAGCAGGTGTTCCCCACGGCTGAGG |
| | | CATCTGAACATTAAGCATATCCCTCTGAGAACCAGCCTGCATTGATACTC |
| | | TTTCTAATGTGGACAGCATCAAGCTATGTACGTAGTTCTGTGCTCAGCAA |
| | | AAGCCCTGACTTCTTTTTGTTTATGTCCTAGCCCCATACAACAAAATCAA |
| | | CCAAAGAATTTTGGTTGTGGATCCAGTCACCTCTGAACATGAACTGACAT |
| | | GTCAGGCTGAGGGCTACCCCAAGGCCGAAGTCATCTGGACAAGCAGTGAC |
| | | CATCAAGTCCTGAGTGGTAAGACCACCACCACCAATTCCAAGAGAGAGGA |
| | | GAAGCTTTTCAATGTGACCAGCACACTGAGAATCAACACAACAACTAATG |
| | | AGATTTTCTACTGCACTTTTAGGAGATTAGATCCTGAGGAAAACCATACA |
| | | GCTGAATTGGTCATCCCAGGTAATATTCTGAATGTGTCCATTAAAATATG |
| | | TCTAACACTGTCCCCTAGCACCTAGCATGATGTCTGCCTATCATAGTCAT |
| | | TCAGTGATTGTTGAATAAATGAATGAATGAATAACACTATGTTTACAAAA |
| | | TATATCCTAATTCCTCACCTCCATTCATCCAAACCATATTGTTACTTAAT |
| | | AAACATTCAGCAGATATTTATGGAATATACCTTTTGTTCCATGCATTGTA |
| | | GTACTCATTGGATACACATAGAATAATAAGACTCAGTTCACACTCTTCAG |
| | | GAAACAGATAAAAAACTAAGAAACAAACAAAAAACAGGCAATCCAACACC |
| | | ATGTGGGAAATGCTTTCATAGCCGGGAAACCTGGGGAATACCTGAGAGGA |
| | | ATACTCAATTCAGGCCTTGTTTCAGGAATCCAAATCCTGGCACATCAGAG |
| | | CTGCTTCCCTCTTTCCAGGGTGGCAGGAAATAAATGGAACATATTTTTCT |
| | | ATCTTATGCCAAACATGAGGGACCCTTTCTCCCCGGTGCCTCTCCCAAGG |
| | | TAGTCTACAATATTTCAACTCTAGCAGTCTGCTTAGTGCATAGAACATGA |
| | | GGCTGTGTGTCCCTGGGCAAATTACTAGACTTCTGTGTGCTTCACTTTCC |
| | | CTGTAGGATTATAATCTACTGAGCAAGCTTATTGTAAGGGTCAGATTAGC |
| | | AACAGTGTATGAAAATGATTTGAGACCATTGCCTGCACAAATTCAACTAT |
| | | TTTTTTTTATCTCACTACTCTACAGAAGTAGGTAGGGTGGGAGACAGAGT |
| | | CTGATGAGAGGCTCAGAATGTGAAAGAAAGTGAGGCGAGTGAGCATGATA |
| | | TTTAATATAAACACAAAGATATTCTGAGAAGAGCTGCTCACTGCCCCCTC |
| | | CCCCAATACATGTTGATAGGAAAATGCCACGTACTTCAGCAAAAACAACT |
| | | GAAAAATTAGATAGAAAAGTCAATCAATAGGAAAAGATAATCCAGGACGG |
| | | TGTTGTGAACAGAAAGAGGGGGAAAAAACTTTAGAAAATGATGGGGATGC |
| | | TCTTACTGGGGTACGAGTCCTCAGGTATTGAACTGGCTTTCAGTAAAAGC |
| | | TAGATTAGTGGGTTCCTGCCATTTACAAGCTGTTTTATGACAACTTACTT |
| | | GTTGGGTGGCCTACAGTAACTCACCTAACTGCACTGAGTCTGTTTCCTCA |
| | | TCTGTAAATTGGGGATTTTTTTTTAAATACCTGGCATGCCTAACTCATAA |
| | | AGTTGTTCTGAAACTGAAATAAAACATACGTGAACAGGCATTGTAAACTG |
| | | TAAGTTACGGAAAAAGCTGGCTGTTGTTGTGTCTTTAAAGTTTCACCTGG |
| | | GTAGTCAAAGATGGATCATGGGTCTCAGTGGAGAGCTGAGCCAGGCAGGA |
| | | GCTGACTAAGGGTGAGAGGTGGGAGTTAGCAGCCTCTGAACATCTGTGTA |
| | | CCATGGGACCCCCTTTCCTCCTGCATGGTACCCCAGACAAGGAGCCTAGT |
| | | AAGAGATACTAATGGCTTGTTGTCCAGAGATGTTCAAACTGCAGAGAAAG |
| | | ATAAGACAACAAGCATTGGCCTCCAATCATGATGACAGATAGGAGGAGGT |
| | | GGGAGCTCCTTAGCAGTGCTGGTTGGCCTTCCATGTTCTACTGTGGGCCA |
| | | TCTCTGCCATGTACTGTAGGCTACTAGCTTCTATATTAAAGAATGCAAGA |
| | | GGGGCCAGGAGCGGAGGCTCATGCCTGTAATCTCAGCACTTTGGGAGGCC |
| | | AAGGTGGGCAGATCACTTGAGGTCAGGAGTTTGTGACCAGCCTGGCCAAC |
| | | ATGGTGAAACTCTGCCTTTACTAAAAATATAAAAATTAGCTGGGTGTGGT |
| | | GGTGTGCACCTGTAATCCCAGCTACTCGGGAGACTGAGGCACAAGAATTG |
| | | CTTGAACCTGGGAGGCGGAAGTTGCAGTGAGCCCAGATTGCGCCACTGCA |
| | | CTCCACCCTGGGCAACAGAGAAAGACTCTGCCTCAAAAAAAAAAAAAAAA |
| | | AGCAAGAGGAAGTGAAATAATCAAGGCCGCCATTTAATAGTGAGCAGCCA |
| | | CTCCATGTGGTACTGTGCAAGCACATTATAAATATTAGCCTCACAAGAAA |
| | | TGTATTAGCATTTGTATTTTGTACACTGGTTAAGTATCTTGCCCAAGACC |
| | | TCAAAACTGGTTAAGGGCAGCAGAATTTAGCCCCAGCACCACCTTTTCAA |
| | | AGCCTGGGCTTCTCACACTTCTCCATGCTGTTCCCATTTTAACACAGGTA |
| | | TCTCGCCATTCCAGCCACTCAAACTTTGGCATTTAAGAAAATTATCCTAA |
| | | AGCTAAACTAAACTTCAAGGATGACCATTCTCCTGACCCCTTCCCATCAA |
| | | AATTTTATCTTTAGTCAGTTTGTTTTCGTTTTGTTTTGTTTTTCAGAACT |
| | | ACCTCTGGCACATCCTCCAAATGAAAGGACTCACTTGGTAATTCTGGGAG |
| | | CCATCTTATTATGCCTTGGTGTAGCACTGACATTCATCTTCCGTTTAAGA |
| | | AAAGGTAGTATTTCCTTAATTGCAGTGGTCTCCACTGGGGGTGAGGAAGG |
| | | GGTGAGAATTGGATCATGGCTGCAAGGAAACCCGACTTAACCTCTGCAAG |
| | | GTGGTGCAAAGGCATTCCACTGTTCAACAGCAATTATATTGAAGCTGAGT |
| | | GGGATCACTGGGTGAAGATGAAGCGTAAGGGGTGAGGGGCAGGAGAATGG |
| | | GTATGGATGGAGGTAGAAGATGCAGTGTCATACAGTTTTTTTCTATCATG |
| | | AAAATAACCACAGACTTACAGAAGAGAAAGAGCTAAAATGCCCGTCATTT |
| | | TCAGTTGCATTTTAGTCTTGCATTAGTTGCAACCAGCTGGTTTCTGGGTA |
| | | CCCTAAGTAATAAAAATAGTTCCTCTGTAGAACTGTAGTATGTTTACCAT |
| | | AGAGTATTTTGCAAAATTTTTGGTAGAGGATGTTACATAATTTGCATGTG |
| | | TTCATTTCTCCATTTACCTGTGGGAACAATTAAAATCCAGGAAAATGAGT |
| | | ATATTCAAATAATTTCCTCCCATTTAAGATGAGTCAGAGTAAATAATTCC |
| | | TCCAATACTTAGAGAAGTATACCAAGAGATCCAGTGATGGTATAGAGTTG |
| | | TCTGATGTTAAATAGGGAAGTAGAATATGGAAGGGGATTCCAATAGTCGT |
| | | TGAAAAATTCCCCATAACCCCTTACATGGGGGAAAGTAGTGTTAACTGAG |
| | | AGAGTAGAGATAAGCTGTTTCCAAAAATTATATTCTTAACAGGACTGAGA |
| | | TAGCCAGAATATAAGGATCAAGTTTCAATGACAGTAAGATCCTGAGATGG |
| | | AGTTGATTTGCACAAAGAAATAATTGTTGCCAGCATGCATTTTGAATATT |
| | | TCTCTGGAAAAAAAGATTAGTTGGCAGTAGAAATGGATAGAAATCAATAG |
| | | ATATTAAAATACCTCAGAATTTGGTTCATCTCTGGGAAAAGATGAAAAAT |
| | | AAAAGTGTATACTCCTCAAGAACATCTAGGATCAAAAGCATGTGCCCTAC |
| | | ACTATTGAATTAATTAACCTCATAAGTTGGGACCTGTGGAATAAGGATGT |
| | | CCACCAGACTTCCTAGGGATTACAAATGTTTCACAGAACTTGAAATTTAA |
| | | ACTTGGGTCACTGTATGGGATGTAGAGCTGTGCTATATGGAAATAAAAAT |
| | | GATTTCTTTTTCTCAAGGGAGAATGATGGATGTGAAAAAATGTGGCATCC |
| | | AAGATACAAACTCAAAGAAGCAAAGTGGTAAGAATATCAGAAGGAATTGG |
| | | GAAGTAAAAGTCAAAGGAAACAAAAAGCTAAAGCAATAACAAAGAGAAAT |
| | | CCATCAGTCATAATCTCCTCTCCTTTTAAAGAATGCTGGTTCCCCTTTGC |
| | | CTCACAGCTAACACAAGAACTCCTCCACCGTCTGAGGAGGTTTAGGAGCA |
| | | GGGAAGGGGAAGGAGTCAGCTTCATTTGCTAATCTTCTGTTGCCCTGCAC |
| | | CCTAGCAGCTCCTTGCAGCAGGGGACAAGGATGACTTAGGTGGATGGATA |
| | | ATTAATTGATTCTAAAATATTGTGTGTCAGTATTGTAATACTATGTTAAT |
| | | TGCACCATGCACGGTATCTCATTTAATCCCCCACCCCTTGCCATTACCAA |
| | | AGAGAGAGAGAGAGAGAGAGAGAGAAATACTAGAATTTATCCTCATTTTA |
| | | CAGTAGAGAAAACAGAGGGTCAAGAAGATAATGTAAAGTGCCCAAGAACA |
| | | CACAGCTGATCACAAAAATCAAGCTTGGGGGCCATTAGCCTAACCACAGA |
| | | CCCTTACTCTTAACCCATCTGCTTCAATCCATTTTGCTACAAATGTTTAC |
| | | ATTTATAAGCAGGGCAGAAAAACCTCATCCAGGTTATTGAACTAAGAAGA |
| | | AAGTTATATTAAGGTTTCTAATTTTTTTAATGTAGTTAGAAACCAAACTT |
| | | AACAATGAGCCCAAGTTTAAAGCAGTCTAATTAACCTGGACAAGCTCAGG |
| | | CAAGTTTCATTCTGTGGCCCATAGCATCATCTGTGTTGTAAAGCTAAGTA |
| | | GCAAATGTTGTTTGGGTCATGCTGGGGGACAAGCCATCCCAATTTGCTCA |
| | | GGACTGAGGGGTTTTCCAGGATATCATGTAAGGATAATTGGGTACAAATA |
| | | TAACCTGCTGCTTTCTCTCATTTCAAATTTATCATTTATCATATCAGCAA |
| | | CTATGAGTTATGTTTTTTATTAGATTTCTTGTTACTTTTTCCCCAGACCA |
| | | CTTCCCATGAAATTAATATACTATTATCACTCTCCAGATACACATTTGGA |
| | | GGAGACGTAATCCAGCATTGGAACTTCTGATCTTCAAGCAGGGATTCTCA |
| | | ACCTGTGGTTTAGGGGTTCATCGGGGCTGAGCGTGACAAGAGGAAGGAAT |
| | | GGGCCCGTGGGATGCAGGCAATGTGGGACTTAAAAGGCCCAAGCACTGAA |
| | | AATGGAACCTGGCGAAAGCAGAGGAGGAGAATGAAGAAAGATGGAGTCAA |
| | | ACAGGGAGCCTGGAGGGAGACCTTGATACTTTCAAATGCCTGAGGGGCTC |
| | | ATCGACGCCTGTGACAGGGAGAAAGGATACTTCTGAACAAGGAGCCTCCA |
| | | AGCAAATCATCCATTGCTCATCCTAGGAAGACGGGTTGAGAATCCCTAAT |
| | | TTGAGGGTCAGTTCCTGCAGAAGTGCCCTTTGCCTCCACTCAATGCCTCA |
| | | ATTTGTTTTCTGCATGACTGAGAGTCTCAGTGTTGGAACGGGACAGTATT |
| | | TATGTATGAGTTTTTCCTATTTATTTTGAGTCTGTGAGGTCTTCTTGTCA |
| | | TGTGAGTGTGGTTGTGAATGATTTCTTTTGAAGATATATTGTAGTAGATG |
| | | TTACAATTTTGTCGCCAAACTAAACTTGCTGCTTAATGATTTGCTCACAT |
| | | CTAGTAAAACATGGAGTATTTGTAAGGTGCTTGGTCTCCTCTATAACTAC |
| | | AAGTATACATTGGAAGCATAAAGATCAAACCGTTGGTTGCATAGGATGTC |
| | | ACCTTTATTTAACCCATTAATACTCTGGTTGACCTAATCTTATTCTCAGA |
| | | CCTCAAGTGTCTGTGCAGTATCTGTTCCATTTAAATATCAGCTTTACAAT |
| | | TATGTGGTAGCCTACACACATAATCTCATTTCATCGCTGTAACCACCCTG |
| | | TTGTGATAACCACTATTATTTTACCCATCGTACAGCTGAGGAAGCAAACA |
| | | GATTAAGTAACTTGCCCAAACCAGTAAATAGCAGACCTCAGACTGCCACC |
| | | CACTGTCCTTTTATAATACAATTTACAGCTATATTTTACTTTAAGCAATT |
| | | CTTTTATTCAAAAACCATTTATTAAGTGCCCTTGCAATATCAATCGCTGT |
| | | GCCAGGCATTGAATCTACAGATGTGAGCAAGACAAAGTACCTGTCCTCAA |
| | | GGAGCTCATAGTATAATGAGGAGATTAACAAGAAAATGTATTATTACAAT |
| | | TTAGTCCAGTGTCATAGCATAAGGATGATGCGAGGGGAAAACCCGAGCAG |
| | | TGTTGCCAAGAGGAGGAAATAGGCCAATGTGGTCTGGGACGGTTGGATAT |
| | | ACTTAAACATCTTAATAATCAGAGTAATTTTCATTTACAAAGAGAGGTCG |
| | | GTACTTAAAATAACCCTGAAAAATAACACTGGAATTCCTTTTCTAGCATT |
| | | ATATTTATTCCTGATTTGCCTTTGCCATATAATCTAATGCTTGTTTATAT |
| | | AGTGTCTGGTATTGTTTAACAGTTCTGTCTTTTCTATTTAAATGCCACTA |
| | | AATTTTAAATTCATACCTTTCCATGATTCAAAATTCAAAAGATCCCATGG |
| | | GAGATGGTTGGAAAATCTCCACTTCATCCTCCAAGCCATTCAAGTTTCCT |
| | | TTCCAGAAGCAACTGCTACTGCCTTTCATTCATATGTTCTTCTAAAGATA |
| | | GTCTACATTTGGAAATGTATGTTAAAAGCACGTATTTTTAAAATTTTTTT |
| | | CCTAAATAGTAACACATTGTATGTCTGCTGTGTACTTTGCTATTTTTATT |
| | | TATTTTAGTGTTTCTTATATAGCAGATGGAATGAATTTGAAGTTCCCAGG |
| | | GCTGAGGATCCATGCCTTCTTTGTTTCTAAGTTATCTTTCCCATAGCTTT |
| | | TCATTATCTTTCATATGATCCAGTATATGTTAAATATGTCCTACATATAC |
| | | ATTTAGACAACCACCATTTGTTAAGTATTTGCTCTAGGACAGAGTTTGGA |
| | | TTTGTTTATGTTTGCTCAAAAGGAGACCCATGGGCTCTCCAGGGTGCACT |
| | | GAGTCAATCTAGTCCTAAAAAGCAATCTTATTATTAACTCTGTATGACAG |
| | | AATCATGTCTGGAACTTTTGTTTTCTGCTTTCTGTCAAGTATAAACTTCA |
| | | CTTTGATGCTGTACTTGCAAAATCACATTTTCTTTCTGGAAATTCCGGCA |
| | | GTGTACCTTGACTGCTAGCTACCCTGTGCCAGAAAAGCCTCATTCGTTGT |
| | | GCTTGAACCCTTGAATGCCACCAGCTGTCATCACTACACAGCCCTCCTAA |
| | | GAGGCTTCCTGGAGGTTTCGAGATTCAGATGCCCTGGGAGATCCCAGAGT |
| | | TTCCTTTCCCTCTTGGCCATATTCTGGTGTCAATGACAAGGAGTACCTTG |
| | | GCTTTGCCACATGTCAAGGCTGAAGAAACAGTGTCTCCAACAGAGCTCCT |
| | | TGTGTTATCTGTTTGTACATGTGCATTTGTACAGTAATTGGTGTGACAGT |
| | | GTTCTTTGTGTGAATTACAGGCAAGAATTGTGGCTGAGCAAGGCACATAG |
| | | TCTACTCAGTCTATTCCTAAGTCCTAACTCCTCCTTGTGGTGTTGGATTT |
| | | GTAAGGCACTTTATCCCTTTTGTCTCATGTTTCATCGTAAATGGCATAGG |
| | | CAGAGATGATACCTAATTCTGCATTTGATTGTCACTTTTTGTACCTGCAT |
| | | TAATTTAATAAAATATTCTTATTTATTTTGTTACTTGGTACACCAGCATG |
| | | TCCATTTTCTTGTTTATTTTGTGTTTAATAAAATGTTCAGTTTAACATCC |
| | | CAGTGGAGAAAGTTA |

*represent the break point

1. (canceled)

2. A method of treating natural killer/T-cell lymphoma in a subject, the method comprising
obtaining a sample from the subject;
detecting the presence or absence of at least one JAK3 activating mutation or at least one PD-L1 structural rearrangement;
administering a compound which impedes the PD-1/PD-L1 axis to the subject if at least one JAK3 activating mutation or at least one PD-L1 structural rearrangement is present in the sample.

3. (canceled)

4. The method of claim 2, wherein the compound is selected from the group consisting of nivolumab (opdivo), pembrolizumab (keytruda), atezolizumab (tecentriq), avelumab (bavencio), durvalumab (imfinzi), pidilizumab, AMP-224, AMP-514, PDR001, cemiplimab, and combinations thereof.

5. The method according to claim 2, wherein the JAK3 activating mutation is selected from the group consisting of M511I, A572V, A573V, R657Q, V722I, V674A, L857P, R403H, Q501H, E958K, and combinations thereof.

6. The method according to claim 5, wherein the JAK3 activating mutation is A572V.

7. The method according to claim 2, wherein the PD-L1 structural rearrangement is a mutation in the PD-L1 gene.

8. The method of claim 7, wherein the mutation is selected from the group consisting of insertions, deletions, substitutions, translocations, inversions, micro-inversions, duplications, tandem repeats, breakpoint(s) (mutations), and combinations thereof.

9. The method of claim 7, wherein the mutation in the PD-L1 gene disrupts the 3′ UTR of the PD-L1 gene.

10. A method of treating natural killer/T-cell lymphoma in a subject, the method comprising administering to a subject an inhibitor selected from the group consisting of PD-1 inhibitor, CD279 inhibitor, PD-L1 inhibitor, CD274 inhibitor and combinations thereof.

11. The method of claim 10, further comprising administration of pembrolizumab.

12. The method of claim 2, wherein the natural killer/T-cell lymphoma is extranodal natural killer/T-cell lymphoma.

13. The method of claim 2, wherein the natural killer/T-cell lymphoma is relapsed and/or refractory natural killer/T-cell lymphoma.

14. A kit for detecting the presence or absence of at least one JAK3 activating mutation or at least one PD-L1 structural rearrangement comprising a detection agent, and at least one pair of primers; wherein the primers enrich for the genomic regions of the JAK3 and PD-L1 genes.

15. The kit of claim 14, wherein the at least pair of primers are selected from the group consisting of SEQ ID NO: 1 and 2, SEQ ID NO: 3 and 4, SEQ ID NO: 5 and 6, SEQ ID NO: 7 and 8, SEQ ID NO: 9 and 10, SEQ ID NO: 11 and 12, SEQ ID NO: 13 and 14, SEQ ID NO: 15 and 16, SEQ ID NO: 17 and 18, SEQ ID NO: 19 and 20, SEQ ID NO: 21 and 22, SEQ ID NO: 23 and 24, SEQ ID NO: 25 and 26, SEQ ID NO: 27 and 28, SEQ ID NO: 29 and 30, SEQ ID NO: 31 and 32, SEQ ID NO: 33 and 34, SEQ ID NO: 35 and 36, SEQ ID NO: 37 and 38, SEQ ID NO: 39 and 40, SEQ ID NO: 41 and 42, SEQ ID NO: 43 and 44, SEQ ID NO: 45 and 46, and SEQ ID NO: 47 and 48.

16-17. (canceled)

18. The method of claim 10, wherein the PD-1 inhibitor is a therapeutically effective amount of pembrolizumab.

19. The method of claim 10, wherein the natural killer/T-cell lymphoma is relapsed and/or refractory natural killer/T-cell lymphoma.

20. The method of claim 10, wherein the subject is characterized by the presence of at least one JAK3-activating mutation or at least one PD-L1 structural rearrangement.


SEQUENCE LISTING (78 sequences)

SEQ ID NO: 1 - DNA, 22 bp, Artificial Sequence
  NKTL1-inversion forward primer
acataaatactgtcccgttcca

SEQ ID NO: 2 - DNA, 20 bp, Artificial Sequence
  NKTL1-inversion reverse primer
gaggctccttgttcagaagt

SEQ ID NO: 3 - DNA, 19 bp, Artificial Sequence
  NKTL1-inversion forward primer
tgtcacaggcgtcgatgag

SEQ ID NO: 4 - DNA, 22 bp, Artificial Sequence
  NKTL1-inversion reverse primer
caaccacactcacatgacaaga

SEQ ID NO: 5 - DNA, 23 bp, Artificial Sequence
  NKTL26-duplication forward primer
cagatacacatttggaggagacg

SEQ ID NO: 6 - DNA, 20 bp, Artificial Sequence
  NKTL26-duplication reverse primer
ctttggccctgtttgtgtcc

SEQ ID NO: 7 - DNA, 24 bp, Artificial Sequence
  NKTL28-translocation forward primer
attcaagtttcctttccagaagca

SEQ ID NO: 8 - DNA, 25 bp, Artificial Sequence
  NKTL28-translocation reverse primer
aagacttttggttggtattttctgt

SEQ ID NO: 9 - DNA, 23 bp, Artificial Sequence
  NKTL31-translocation forward primer
ccatgcacggtatctcatttaat

SEQ ID NO: 10 - DNA, 22 bp, Artificial Sequence
  NKTL31-translocation reverse primer
tcagtatctcatcccacctgac

SEQ ID NO: 11 - DNA, 19 bp, Artificial Sequence
  NKTL29-JAK3-missense forward primer
ggggctctcactgtctcca

SEQ ID NO: 12 - DNA, 22 bp, Artificial Sequence
  NKTL29-JAK3-missense reverse primer
aagaaacccacgcatcttctct

SEQ ID NO: 13 - DNA, 19 bp, Artificial Sequence
  NKTL30-JAK3-missense forward primer
ggggctctcactgtctcca

SEQ ID NO: 14 - DNA, 22 bp, Artificial Sequence
  NKTL30-JAK3-missense reverse primer
aagaaacccacgcatcttctct

SEQ ID NO: 15 - DNA, 20 bp, Artificial Sequence
  NKTL27-ARID1B-insertion forward primer
catgtgctgtgactgcttgt

SEQ ID NO: 16 - DNA, 21 bp, Artificial Sequence
  NKTL27-ARID1B-insertion reverse primer
cctcttcctacagtactcccc

SEQ ID NO: 17 - DNA, 21 bp, Artificial Sequence
  NKTL4-deletion forward primer
caagtttcattctgtggccca

SEQ ID NO: 18 - DNA, 20 bp, Artificial Sequence
  NKTL4-deletion reverse primer
ttgggtcaaagcggaatgtg

SEQ ID NO: 19 - DNA, 23 bp, Artificial Sequence
  NKTL6-complex forward primer
cagatacacatttggaggagacg

SEQ ID NO: 20 - DNA, 20 bp, Artificial Sequence
  NKTL6-complex reverse primer
atggatagggctgcaggtga

SEQ ID NO: 21 - DNA, 20 bp, Artificial Sequence
  NKTL11-duplication forward primer
gctcatcctaggaagacggg

SEQ ID NO: 22 - DNA, 20 bp, Artificial Sequence
  NKTL11-duplication reverse primer
aactggagtcgaaggtcaca

SEQ ID NO: 23 - DNA, 24 bp, Artificial Sequence
  NKTL15-duplication forward primer
gagaaaacagagggtcaagaagat

SEQ ID NO: 24 - DNA, 24 bp, Artificial Sequence
  NKTL15-duplication reverse primer
gaaaccaaaagcaagcaggagtag

SEQ ID NO: 25 - DNA, 23 bp, Artificial Sequence
  NKTL16-translocation forwrd primer
tccctgacaattctaaatcgagt

SEQ ID NO: 26 - DNA, 20 bp, Artificial Sequence
  NKTL16-translocation reverse primer
acccgacttaacctctgcaa

SEQ ID NO: 27 - DNA, 20 bp, Artificial Sequence
  NKTL17-deletion forward primer
gcccgtcattttcagttgca

SEQ ID NO: 28 - DNA, 20 bp, Artificial Sequence
  NKTL17-deletion reverse primer
caggagaatggcgtgaactc

SEQ ID NO: 29 - DNA, 24 bp, Artificial Sequence
  NKTL35-deletion forward primer
ccagaccacttcccatgaaattaa

SEQ ID NO: 30 - DNA, 24 bp, Artificial Sequence
  NKTL35-deletion reverse primer
tactcatacatttggcctcagttg

SEQ ID NO: 31 - DNA, 24 bp, Artificial Sequence
  NKTL37-inversion forward primer
tataccaagagatccagtgatggt

SEQ ID NO: 32 - DNA, 24 bp, Artificial Sequence
  NKTL37-inversion reverse primer
aatcatgtttcagtaccattggct

SEQ ID NO: 33 - DNA, 24 bp, Artificial Sequence
  NKTL37-inversion forward primer
ctactctccagcccatctattgag

SEQ ID NO: 34 - DNA, 22 bp, Artificial Sequence
  NKTL37-inversion reverse primer
atctattgagggctgatctggg

SEQ ID NO: 35 - DNA, 21 bp, Artificial Sequence
  NKTL4-deletion forward primer
acttggtaattctgggagcca

SEQ ID NO: 36 - DNA, 20 bp, Artificial Sequence
  NKTL4-deletion reverse primer
ccaccttctgaacagtgacc

SEQ ID NO: 37 - DNA, 21 bp, Artificial Sequence
  NKTL6-complex forward primer
acttggtaattctgggagcca

SEQ ID NO: 38 - DNA, 20 bp, Artificial Sequence
  NKTL6-complex reverse primer
ggccagctgaggtctttatt

SEQ ID NO: 39 - DNA, 30 bp, Artificial Sequence
  NKTL15-duplication forward primer
caacacaacaactaatgagattttctactg

SEQ ID NO: 40 - DNA, 26 bp, Artificial Sequence
  NKTL15-duplication reverse primer
cctcccatgacatctctcctcttatg

SEQ ID NO: 41 - DNA, 26 bp, Artificial Sequence
  NKTL16-translocation forward primer
aatgaaaggactcacttggtaattct

SEQ ID NO: 42 - DNA, 26 bp, Artificial Sequence
  NKTL16-translocation reverse primer
gttttgcagctagaattcagttgtaa

SEQ ID NO: 43 - DNA, 22 bp, Artificial Sequence
  NKTL17-deletion forward primer
cctccaaatgaaaggactcact

SEQ ID NO: 44 - DNA, 22 bp, Artificial Sequence
  NKTL17-deletion reverse primer
gcaggtttggcaattctgattc

SEQ ID NO: 45 - DNA, 24 bp, Artificial Sequence
  NKTL35-deletion forward primer
cagcattggaacttctgatcttca

SEQ ID NO: 46 - DNA, 23 bp, Artificial Sequence
  NKTL35-deletion reverse primer
ggcctcagttgtcacagtatttt

SEQ ID NO: 47 - DNA, 25 bp, Artificial Sequence
  NKTL37-inversion forward primer
tgaaaggactcacttggtaattctg

SEQ ID NO: 48 - DNA, 24 bp, Artificial Sequence
  NKTL37-inversion reverse primer
aggtataataatgctgcctgagat

SEQ ID NO: 49 - DNA, 32 bp, Artificial Sequence
  CD274-3UTR_XhoI_F primer
cgtagtctcgagtccagcattggaacttctga

SEQ ID NO: 50 - DNA, 34 bp, Artificial Sequence
  CD274-3UTR_NotI_R primer
caattagcggccgcaactttctccactgggatgt

SEQ ID NO: 51 - DNA, 28 bp, Artificial Sequence
  CD274-3UTR_F primer
tccagcattggaacttctgatcttcaag

SEQ ID NO: 52 - DNA, 35 bp, Artificial Sequence
  CD274-A-R primer
tgactgagagtctcaaggtctccctccaggctccc

SEQ ID NO: 53 - DNA, 35 bp, Artificial Sequence
  Inv-overhang-F primer
cctggagggagaccttgagactctcagtcatgcag

SEQ ID NO: 54 - DNA, 35 bp, Artificial Sequence
  Inv-overhang-R primer
gtcccgttccaacactgatactttcaaatgcctga

SEQ ID NO: 55 - DNA, 35 bp, Artificial Sequence
  CD274-C-F and F2 primer
catttgaaagtatcagtgttggaacgggacagtat

SEQ ID NO: 56 - DNA, 27 bp, Artificial Sequence
  CD274-3UTR_R primer
aactttctccactgggatgttaaactg

SEQ ID NO: 57 - DNA, 28 bp, Artificial Sequence
  CD274-3UTR_F primer
tccagcattggaacttctgatcttcaag

SEQ ID NO: 58 - DNA, 35 bp, Artificial Sequence; identical to SEQ ID NO: 54
  Inv-overhang-R primer

SEQ ID NO: 59 - DNA, 32 bp, Artificial Sequence; identical to SEQ ID NO: 49
  CD274-3UTR_XhoI_F primer

SEQ ID NO: 60 - DNA, 34 bp, Artificial Sequence; identical to SEQ ID NO: 50
  CD274-3UTR_NotI_R primer

SEQ ID NO: 61 - DNA, 34 bp, Artificial Sequence
  NKTL1 validated Sanger sequence
  misc_feature (17)..(18): breakpoint mutation between referenced residues
ggcatttgaaagtatcagtgttggaacgggacag

SEQ ID NO: 62 - DNA, 40 bp, Artificial Sequence
  NKTL11 validated Sanger sequence
  misc_feature (20)..(21): breakpoint mutation between referenced residues
tgtcatgtgagtgtggttgtgaacagttcctgaactctga

SEQ ID NO: 63 - DNA, 40 bp, Artificial Sequence
  NKTL15 validated Sanger sequence
  misc_feature (20)..(21): breakpoint mutation between referenced residues
taagaagaaagttatattataatatagtttgcttttacaa

SEQ ID NO: 64 - DNA, 40 bp, Artificial Sequence
  NKTL6 (complex case) 1 validated Sanger sequence
  misc_feature (20)..(21): breakpoint mutation between referenced residues
agcgtgacaagaggaaggaatgtgccaccatgcccagcta

SEQ ID NO: 65 - DNA, 40 bp, Artificial Sequence
  NKTL6 (complex case) 2 validated Sanger sequence
  misc_feature (20)..(21): breakpoint mutation between referenced residues
cgtattggccaggatagtctagaaaattttgctaaagcag

SEQ ID NO: 66 - DNA, 40 bp, Artificial Sequence
  NKTL4 validated Sanger sequence
  misc_feature (20)..(21): breakpoint mutation between referenced residues
tgtgttgtaaagctaagtagctcaggtactttgctatccc

SEQ ID NO: 67 - DNA, 40 bp, Artificial Sequence
  NKTL17 validated Sanger sequence
  misc_feature (20)..(21): breakpoint mutation between referenced residues
catttaagatgagtcagagttttttgagacggagtctcgc

SEQ ID NO: 68 - DNA, 40 bp, Artificial Sequence
  NKTL16 validated Sanger sequence
  misc_feature (20)..(21): breakpoint mutation between referenced residues
caggagaatgggtatggatgagaacacatacttcctctcc

SEQ ID NO: 69 - DNA, 41 bp, Artificial Sequence
  NKTL26 validated Sanger sequence
  misc_feature (21)..(22): breakpoint mutation between referenced residues
ctgatcttcaagcaggggattgatgtgctttgttaaacaga

SEQ ID NO: 70 - DNA, 40 bp, Artificial Sequence
  NKTL28 validated Sanger sequence
  misc_feature (20)..(21): breakpoint mutation between referenced residues
atgttaaaagcacgtattttgaataaaatgttactttgtc

SEQ ID NO: 71 - DNA, 40 bp, Artificial Sequence
  NKTL31 validated Sanger sequence
  misc_feature (20)..(21): breakpoint mutation between referenced residues
ctccctccctttctctctctctctctctctttggtaatgg

SEQ ID NO: 72 - DNA, 40 bp, Artificial Sequence
  FLN375 validated Sanger sequence
  misc_feature (20)..(21): breakpoint mutation between referenced residues
cgtgggatgcaggcaatgtggaatataacaaataaagcaa

SEQ ID NO: 73 - DNA, 40 bp, Artificial Sequence
  FLN377 validated Sanger sequence
  misc_feature (20)..(21): breakpoint mutation between referenced residues
aatatggaaggggattccaaatctgaagggacctcagggg

SEQ ID NO: 74 - DNA, 3375 bp, Artificial Sequence
  JAK3 cDNA wild type
atggcacctccaagtgaagagacgcccctgatccctcagcgttcatgcagcctcttgtccacggaggctggtgccctgcatgtgctgctgcccgctcggggccccgggcccccccagcgcctatctttctcctttggggaccacttggctgaggacctgtgcgtgcaggctgccaaggccagcggcatcctgcctgtgtaccactccctctttgctctggccacggaggacctgtcctgctggttccccccgagccacatcttctccgtggaggatgccagcacccaagtcctgctgtacaggattcgcttttacttccccaattggtttgggctggagaagtgccaccgcttcgggctacgcaaggatttggccagtgctatccttgacctgccagtcctggagcacctctttgcccagcaccgcagtgacctggtgagtgggcgcctccccgtgggcctcagtctcaaggagcagggtgagtgtctcagcctggccgtgttggacctggcccggatggcgcgagagcaggcccagcggccgggagagctgctgaagactgtcagctacaaggcctgcctacccccaagcctgcgcgacctgatccagggcctgagcttcgtgacgcggaggcgtattcggaggacggtgcgcagagccctgcgccgcgtggccgcctgccaggcagaccggcactcgctcatggccaagtacatcatggacctggagcggctggatccagccggggccgccgagaccttccacgtgggcctccctggggcccttggtggccacgacgggctggggctgctccgcgtggctggtgacggcggcatcgcctggacccagggagaacaggaggtcctccagcccttctgcgactttccagaaatcgtagacattagcatcaagcaggccccgcgcgttggcccggccggagagcaccgcctggtcactgttaccaggacagacaaccagattttagaggccgagttcccagggctgcccgaggctctgtcgttcgtggcgctcgtggacggctacttccggctgaccacggactcccagcacttcttctgcaaggaggtggcaccgccgaggctgctggaggaagtggccgagcagtgccacggccccatcactctggactttgccatcaacaagctcaagactgggggctcacgtcctggctcctatgttctccgccgcagcccccaggactttgacagcttcctcctcactgtctgtgtccagaacccccttggtcctgattataagggctgcctcatccggcgcagccccacaggaaccttccttctggttggcctcagccgaccccacagcagtcttcgagagctcctggcaacctgctgggatggggggctgcacgtagatggggtggcagtgaccctcacttcctgctgtatccccagacccaaagaaaagtccaacctgatcgtggtccagagaggtcacagcccacccacatcatccttggttcagccccaatcccaataccagctgagtcagatgacatttcacaagatccctgctgacagcctggagtggcatgagaacctgggccatgggtccttcaccaagatttaccggggctgtcgccatgaggtggtggatggggaggcccgaaagacagaggtgctgctgaaggtcatggatgccaagcacaagaactgcatggagtcattcctggaagcagcgagcttgatgagccaagtgtcgtaccggcatctcgtgctgctccacggcgtgtgcatggctggagacagcaccatggtgcaggaatttgtacacctgggggccatagacatgtatctgcgaaaacgtggccacctggtgccagccagctggaagctgcaggtggtcaaacagctggcctacgccctcaactatctggaggacaaaggcctgccccatggcaatgtctctgcccggaaggtgctcctggctcgggagggggctgatgggagcccgcccttcatcaagctgagtgaccctggggtcagccccgctgtgttaagcctggagatgctcaccgacaggatcccctgggtggcccccgagtgtctccgggaggcgcagacacttagcttggaagctgacaagtggggcttcggcgccacggtctgggaagtgtttagtggcgtcaccatgcccatcagtgccctggatcctgctaagaaactccaattttatgaggaccggcagcagctgccggcccccaagtggacagagctggccctgctgattcaacagtgcatggcctatgagccggtccagaggccctccttccgagccgtcattcgtgacctcaatagcctcatctcttcagactatgagctcctctcagaccccacacctggtgccctggcacctcgtgatgggctgtggaatggtgcccagctctatgcctgccaagaccccacgatcttcgaggagagacacctcaagtacatctcacagctgggcaagggcaactttggcagcgtggagctgtgccgctatgacccgctaggcgacaatacaggtgccctggtggccgtgaaacagctgcagcacagcgggccagaccagcagagggactttcagcgggagattcagatcctcaaagcactgcacagtgatttcattgtcaagtatcgtggtgtcagctatggcccgggccgccagagcctgcggctggtcatggagtacctgcccagcggctgcttgcgcgacttcctgcagcggcaccgcgcgcgcctcgatgccagccgcctccttctctattcctcgcagatctgcaagggcatggagtacctgggctcccgccgctgcgtgcaccgcgacctggccgcccgaaacatcctcgtggagagcgaggcacacgtcaagatcgctgacttcggcctagctaagctgctgccgcttgacaaagactactacgtggtccgcgagccaggccagagccccattttctggtatgcccccgaatccctctcggacaacatcttctctcgccagtcagacgtctggagcttcggggtcgtcctgtacgagctcttcacctactgcgacaaaagctgcagcccctcggccgagttcctgcggatgatgggatgtgagcgggatgtccccgccctctgccgcctcttggaactgctggaggagggccagaggctgccggcgcctcctgcctgccctgctgaggttcacgagctcatgaagctgtgctgggcccctagcccacaggaccggccatcattcagcgccctgggcccccagctggacatgctgtggagcggaagccgggggtgtgagactcatgccttcactgctcacccagagggcaaacaccactccctgtccttttcatag

SEQ ID NO: 75 - DNA, 3375 bp, Artificial Sequence
  JAK3 cDNA single mutation 1
atggcacctccaagtgaagagacgcccctgatccctcagcgttcatgcagcctcttgtccacggaggctggtgccctgcatgtgctgctgcccgctcggggccccgggcccccccagcgcctatctttctcctttggggaccacttggctgaggacctgtgcgtgcaggctgccaaggccagcggcatcctgcctgtgtaccactccctctttgctctggccacggaggacctgtcctgctggttccccccgagccacatcttctccgtggaggatgccagcacccaagtcctgctgtacaggattcgcttttacttccccaattggtttgggctggagaagtgccaccgcttcgggctacgcaaggatttggccagtgctatccttgacctgccagtcctggagcacctctttgcccagcaccgcagtgacctggtgagtgggcgcctccccgtgggcctcagtctcaaggagcagggtgagtgtctcagcctggccgtgttggacctggcccggatggcgcgagagcaggcccagcggccgggagagctgctgaagactgtcagctacaaggcctgcctacccccaagcctgcgcgacctgatccagggcctgagcttcgtgacgcggaggcgtattcggaggacggtgcgcagagccctgcgccgcgtggccgcctgccaggcagaccggcactcgctcatggccaagtacatcatggacctggagcggctggatccagccggggccgccgagaccttccacgtgggcctccctggggcccttggtggccacgacgggctggggctgctccgcgtggctggtgacggcggcatcgcctggacccagggagaacaggaggtcctccagcccttctgcgactttccagaaatcgtagacattagcatcaagcaggccccgcgcgttggcccggccggagagcaccgcctggtcactgttaccaggacagacaaccagattttagaggccgagttcccagggctgcccgaggctctgtcgttcgtggcgctcgtggacggctacttccggctgaccacggactcccagcacttcttctgcaaggaggtggcaccgccgaggctgctggaggaagtggccgagcagtgccacggccccatcactctggactttgccatcaacaagctcaagactgggggctcacgtcctggctcctatgttctccgccgcagcccccaggactttgacagcttcctcctcactgtctgtgtccagaacccccttggtcctgattataagggctgcctcatccggcgcagccccacaggaaccttccttctggttggcctcagccgaccccacagcagtcttcgagagctcctggcaacctgctgggatggggggctgcacgtagatggggtggcagtgaccctcacttcctgctgtatccccagacccaaagaaaagtccaacctgatcgtggtccagagaggtcacagcccacccacatcatccttggttcagccccaatcccaataccagctgagtcagatgacatttcacaagatccctgctgacagcctggagtggcatgagaacctgggccatgggtccttcaccaagatttaccggggctgtcgccatgaggtggtggatggggaggcccgaaagacagaggtgctgctgaaggtcatggatgccaagcacaagaactgcatggagtcattcctggaagtagcgagcttgatgagccaagtgtcgtaccggcatctcgtgctgctccacggcgtgtgcatggctggagacagcaccatggtgcaggaatttgtacacctgggggccatagacatgtatctgcgaaaacgtggccacctggtgccagccagctggaagctgcaggtggtcaaacagctggcctacgccctcaactatctggaggacaaaggcctgccccatggcaatgtctctgcccggaaggtgctcctggctcgggagggggctgatgggagcccgcccttcatcaagctgagtgaccctggggtcagccccgctgtgttaagcctggagatgctcaccgacaggatcccctgggtggcccccgagtgtctccgggaggcgcagacacttagcttggaagctgacaagtggggcttcggcgccacggtctgggaagtgtttagtggcgtcaccatgcccatcagtgccctggatcctgctaagaaactccaattttatgaggaccggcagcagctgccggcccccaagtggacagagctggccctgctgattcaacagtgcatggcctatgagccggtccagaggccctccttccgagccgtcattcgtgacctcaatagcctcatctcttcagactatgagctcctctcagaccccacacctggtgccctggcacctcgtgatgggctgtggaatggtgcccagctctatgcctgccaagaccccacgatcttcgaggagagacacctcaagtacatctcacagctgggcaagggcaactttggcagcgtggagctgtgccgctatgacccgctaggcgacaatacaggtgccctggtggccgtgaaacagctgcagcacagcgggccagaccagcagagggactttcagcgggagattcagatcctcaaagcactgcacagtgatttcattgtcaagtatcgtggtgtcagctatggcccgggccgccagagcctgcggctggtcatggagtacctgcccagcggctgcttgcgcgacttcctgcagcggcaccgcgcgcgcctcgatgccagccgcctccttctctattcctcgcagatctgcaagggcatggagtacctgggctcccgccgctgcgtgcaccgcgacctggccgcccgaaacatcctcgtggagagcgaggcacacgtcaagatcgctgacttcggcctagctaagctgctgccgcttgacaaagactactacgtggtccgcgagccaggccagagccccattttctggtatgcccccgaatccctctcggacaacatcttctctcgccagtcagacgtctggagcttcggggtcgtcctgtacgagctcttcacctactgcgacaaaagctgcagcccctcggccgagttcctgcggatgatgggatgtgagcgggatgtccccgccctctgccgcctcttggaactgctggaggagggccagaggctgccggcgcctcctgcctgccctgctgaggttcacgagctcatgaagctgtgctgggcccctagcccacaggaccggccatcattcagcgccctgggcccccagctggacatgctgtggagcggaagccgggggtgtgagactcatgccttcactgctcacccagagggcaaacaccactccctgtccttttcatag

SEQ ID NO: 76 - DNA, 3375 bp, Artificial Sequence
  JAK3 cDNA single mutation 2
atggcacctccaagtgaagagacgcccctgatccctcagcgttcatgcagcctcttgtccacggaggctggtgccctgcatgtgctgctgcccgctcggggccccgggcccccccagcgcctatctttctcctttggggaccacttggctgaggacctgtgcgtgcaggctgccaaggccagcggcatcctgcctgtgtaccactccctctttgctctggccacggaggacctgtcctgctggttccccccgagccacatcttctccgtggaggatgccagcacccaagtcctgctgtacaggattcgcttttacttccccaattggtttgggctggagaagtgccaccgcttcgggctacgcaaggatttggccagtgctatccttgacctgccagtcctggagcacctctttgcccagcaccgcagtgacctggtgagtgggcgcctccccgtgggcctcagtctcaaggagcagggtgagtgtctcagcctggccgtgttggacctggcccggatggcgcgagagcaggcccagcggccgggagagctgctgaagactgtcagctacaaggcctgcctacccccaagcctgcgcgacctgatccagggcctgagcttcgtgacgcggaggcgtattcggaggacggtgcgcagagccctgcgccgcgtggccgcctgccaggcagaccggcactcgctcatggccaagtacatcatggacctggagcggctggatccagccggggccgccgagaccttccacgtgggcctccctggggcccttggtggccacgacgggctggggctgctccgcgtggctggtgacggcggcatcgcctggacccagggagaacaggaggtcctccagcccttctgcgactttccagaaatcgtagacattagcatcaagcaggccccgcgcgttggcccggccggagagcaccgcctggtcactgttaccaggacagacaaccagattttagaggccgagttcccagggctgcccgaggctctgtcgttcgtggcgctcgtggacggctacttccggctgaccacggactcccagcacttcttctgcaaggaggtggcaccgccgaggctgctggaggaagtggccgagcagtgccacggccccatcactctggactttgccatcaacaagctcaagactgggggctcacgtcctggctcctatgttctccgccgcagcccccaggactttgacagcttcctcctcactgtctgtgtccagaacccccttggtcctgattataagggctgcctcatccggcgcagccccacaggaaccttccttctggttggcctcagccgaccccacagcagtcttcgagagctcctggcaacctgctgggatggggggctgcacgtagatggggtggcagtgaccctcacttcctgctgtatccccagacccaaagaaaagtccaacctgatcgtggtccagagaggtcacagcccacccacatcatccttggttcagccccaatcccaataccagctgagtcagatgacatttcacaagatccctgctgacagcctggagtggcatgagaacctgggccatgggtccttcaccaagatttaccggggctgtcgccatgaggtggtggatggggaggcccgaaagacagaggtgctgctgaaggtcatggatgccaagcacaagaactgcatggagtcattcctggaagcagtgagcttgatgagccaagtgtcgtaccggcatctcgtgctgctccacggcgtgtgcatggctggagacagcaccatggtgcaggaatttgtacacctgggggccatagacatgtatctgcgaaaacgtggccacctggtgccagccagctggaagctgcaggtggtcaaacagctggcctacgccctcaactatctggaggacaaaggcctgccccatggcaatgtctctgcccggaaggtgctcctggctcgggagggggctgatgggagcccgcccttcatcaagctgagtgaccctggggtcagccccgctgtgttaagcctggagatgctcaccgacaggatcccctgggtggcccccgagtgtctccgggaggcgcagacacttagcttggaagctgacaagtggggcttcggcgccacggtctgggaagtgtttagtggcgtcaccatgcccatcagtgccctggatcctgctaagaaactccaattttatgaggaccggcagcagctgccggcccccaagtggacagagctggccctgctgattcaacagtgcatggcctatgagccggtccagaggccctccttccgagccgtcattcgtgacctcaatagcctcatctcttcagactatgagctcctctcagaccccacacctggtgccctggcacctcgtgatgggctgtggaatggtgcccagctctatgcctgccaagaccccacgatcttcgaggagagacacctcaagtacatctcacagctgggcaagggcaactttggcagcgtggagctgtgccgctatgacccgctaggcgacaatacaggtgccctggtggccgtgaaacagctgcagcacagcgggccagaccagcagagggactttcagcgggagattcagatcctcaaagcactgcacagtgatttcattgtcaagtatcgtggtgtcagctatggcccgggccgccagagcctgcggctggtcatggagtacctgcccagcggctgcttgcgcgacttcctgcagcggcaccgcgcgcgcctcgatgccagccgcctccttctctattcctcgcagatctgcaagggcatggagtacctgggctcccgccgctgcgtgcaccgcgacctggccgcccgaaacatcctcgtggagagcgaggcacacgtcaagatcgctgacttcggcctagctaagctgctgccgcttgacaaagactactacgtggtccgcgagccaggccagagccccattttctggtatgcccccgaatccctctcggacaacatcttctctcgccagtcagacgtctggagcttcggggtcgtcctgtacgagctcttcacctactgcgacaaaagctgcagcccctcggccgagttcctgcggatgatgggatgtgagcgggatgtccccgccctctgccgcctcttggaactgctggaggagggccagaggctgccggcgcctcctgcctgccctgctgaggttcacgagctcatgaagctgtgctgggcccctagcccacaggaccggccatcattcagcgccctgggcccccagctggacatgctgtggagcggaagccgggggtgtgagactcatgccttcactgctcacccagagggcaaacaccactccctgtccttttcatag

SEQ ID NO: 77 - DNA, 3375 bp, Artificial Sequence
  JAK3 cDNA double mutation
atggcacctccaagtgaagagacgcccctgatccctcagcgttcatgcagcctcttgtccacggaggctggtgccctgcatgtgctgctgcccgctcggggccccgggcccccccagcgcctatctttctcctttggggaccacttggctgaggacctgtgcgtgcaggctgccaaggccagcggcatcctgcctgtgtaccactccctctttgctctggccacggaggacctgtcctgctggttccccccgagccacatcttctccgtggaggatgccagcacccaagtcctgctgtacaggattcgcttttacttccccaattggtttgggctggagaagtgccaccgcttcgggctacgcaaggatttggccagtgctatccttgacctgccagtcctggagcacctctttgcccagcaccgcagtgacctggtgagtgggcgcctccccgtgggcctcagtctcaaggagcagggtgagtgtctcagcctggccgtgttggacctggcccggatggcgcgagagcaggcccagcggccgggagagctgctgaagactgtcagctacaaggcctgcctacccccaagcctgcgcgacctgatccagggcctgagcttcgtgacgcggaggcgtattcggaggacggtgcgcagagccctgcgccgcgtggccgcctgccaggcagaccggcactcgctcatggccaagtacatcatggacctggagcggctggatccagccggggccgccgagaccttccacgtgggcctccctggggcccttggtggccacgacgggctggggctgctccgcgtggctggtgacggcggcatcgcctggacccagggagaacaggaggtcctccagcccttctgcgactttccagaaatcgtagacattagcatcaagcaggccccgcgcgttggcccggccggagagcaccgcctggtcactgttaccaggacagacaaccagattttagaggccgagttcccagggctgcccgaggctctgtcgttcgtggcgctcgtggacggctacttccggctgaccacggactcccagcacttcttctgcaaggaggtggcaccgccgaggctgctggaggaagtggccgagcagtgccacggccccatcactctggactttgccatcaacaagctcaagactgggggctcacgtcctggctcctatgttctccgccgcagcccccaggactttgacagcttcctcctcactgtctgtgtccagaacccccttggtcctgattataagggctgcctcatccggcgcagccccacaggaaccttccttctggttggcctcagccgaccccacagcagtcttcgagagctcctggcaacctgctgggatggggggctgcacgtagatggggtggcagtgaccctcacttcctgctgtatccccagacccaaagaaaagtccaacctgatcgtggtccagagaggtcacagcccacccacatcatccttggttcagccccaatcccaataccagctgagtcagatgacatttcacaagatccctgctgacagcctggagtggcatgagaacctgggccatgggtccttcaccaagatttaccggggctgtcgccatgaggtggtggatggggaggcccgaaagacagaggtgctgctgaaggtcatggatgccaagcacaagaactgcatggagtcattcctggaagtagtgagcttgatgagccaagtgtcgtaccggcatctcgtgctgctccacggcgtgtgcatggctggagacagcaccatggtgcaggaatttgtacacctgggggccatagacatgtatctgcgaaaacgtggccacctggtgccagccagctggaagctgcaggtggtcaaacagctggcctacgccctcaactatctggaggacaaaggcctgccccatggcaatgtctctgcccggaaggtgctcctggctcgggagggggctgatgggagcccgcccttcatcaagctgagtgaccctggggtcagccccgctgtgttaagcctggagatgctcaccgacaggatcccctgggtggcccccgagtgtctccgggaggcgcagacacttagcttggaagctgacaagtggggcttcggcgccacggtctgggaagtgtttagtggcgtcaccatgcccatcagtgccctggatcctgctaagaaactccaattttatgaggaccggcagcagctgccggcccccaagtggacagagctggccctgctgattcaacagtgcatggcctatgagccggtccagaggccctccttccgagccgtcattcgtgacctcaatagcctcatctcttcagactatgagctcctctcagaccccacacctggtgccctggcacctcgtgatgggctgtggaatggtgcccagctctatgcctgccaagaccccacgatcttcgaggagagacacctcaagtacatctcacagctgggcaagggcaactttggcagcgtggagctgtgccgctatgacccgctaggcgacaatacaggtgccctggtggccgtgaaacagctgcagcacagcgggccagaccagcagagggactttcagcgggagattcagatcctcaaagcactgcacagtgatttcattgtcaagtatcgtggtgtcagctatggcccgggccgccagagcctgcggctggtcatggagtacctgcccagcggctgcttgcgcgacttcctgcagcggcaccgcgcgcgcctcgatgccagccgcctccttctctattcctcgcagatctgcaagggcatggagtacctgggctcccgccgctgcgtgcaccgcgacctggccgcccgaaacatcctcgtggagagcgaggcacacgtcaagatcgctgacttcggcctagctaagctgctgccgcttgacaaagactactacgtggtccgcgagccaggccagagccccattttctggtatgcccccgaatccctctcggacaacatcttctctcgccagtcagacgtctggagcttcggggtcgtcctgtacgagctcttcacctactgcgacaaaagctgcagcccctcggccgagttcctgcggatgatgggatgtgagcgggatgtccccgccctctgccgcctcttggaactgctggaggagggccagaggctgccggcgcctcctgcctgccctgctgaggttcacgagctcatgaagctgtgctgggcccctagcccacaggaccggccatcattcagcgccctgggcccccagctggacatgctgtggagcggaagccgggggtgtgagactcatgccttcactgctcacccagagggcaaacaccactccctgtccttttcatag

SEQ ID NO: 78 - DNA, 20065 bp, Artificial Sequence
  PD-L1 gDNA full length, >hg19_dna range=chr95450503-5470567 5′pad=0 3′pad=0 strand=+ repeatMasking=none
ggcgcaacgctgagcagctggcgcgtcccgcgcggccccagttctgcgcagcttcccgaggctccgcaccagccgcgcttctgtccgcctgcaggtagggagcgttgttcctccgcgggtgcccacggcccagtatctctggctagctcgctgggcactttaggacggagggtctctacaccctttctttgggatggagagaggagaagggaaagggaacgcgatggtctagggggcagtagagccaattacctgttggggttaataagaacaggcaatgcatctggccttcctccaggcgcgattcagttttgctctaaaaataatttatacctctaaaaataaataagataggtagtataggataggtagtcattcttatgcgactgtgtgttcagaatatagctctgatgctaggctggaggtctggacacgggtccaagtccaccgccagctgcttgctagtaacatgacttgtgtaagttatcccagctgcagcatctaagtaagtctcttcctgcgctaagcaggtccaggatccctgaacggaatttatttgctctgtccattctgagaacccaaaggagtcctaaaagaggaatggaggagcctaagaataaaaatagtataataaaacatttcttagacacattgaccttggcctatgtcaaagttcagtctgggtttgtcttataacacaaggagtaaaagtaccattgttctacctctttttttaatacttgaaaaaaatttactgtggatgcttttctatgaattaaataaccttctaaaaaatgttttcattgctgcattcgattagattgggtaactaaatgaaattaattcctcactgttgggtataaaggttatttacagtggttctgtcttagccattcactgaactcattgcatatatatctctggaatattgctgattgtttccttcaagtaaacttagaagtgtaactacttagtcaaagagcctgaatattttaaaggccttttgaagaaaactgaaaatgctttccagaaaggatgtatcagttgacaatgacagtcgtcaacagtatttaaggagaactatgatactctgaagaaaaacttagcctttctcagtaaaagtaggtaggcagaggccacatgacagcagttagagtgtggtcttcaaggaagtcacagaaatactgtggggaattgaaaccccatgtggaaaatgtacaagagtgtctcagtgtgactgagaaggaggttgggcatggggtttcatggagtttaataaagtttggtcacttagtagaggtttaataaatcaactgtcttaatctttgatcctacttaagaattttttttttgtttttgtagagatggggctcttgttatgttgcccaggctgttctcgaactcctagcctcaggcgatcctccctcctcaggctccagaagtcctgggattactggcgggagccaccatgcaggcctcttgctcctacttttgagaaaggaagtttaaccggttttttttgtctttttttttttttttttgagacagagtctcactctgttgcccatgctggagtgcagtggtgcaatctcagctcactgcctcccgggttcaagtgattctcctgcctcagcctcccgagtagctgggactacaggcacctgccaccacgcccagctaatttttgtatttttagtagaaatggggtttcaccatattggccaggctgatctcgaactcctgacctcaggtgatccgcctgcctcggcctcccaaagtgctgggattacaggcatgagccactgctcctggctgcttaactttttctctatctcatcctcctacccatcctacccttggaagatagagaagtagtattagttccatagtgttatactgggcttcccccagggacaaacccacttccccaacctgaatgagccatcacttcttccccagtttacatttcattgctctttaaatgtctccattcggatatgggaattcacatatggtcataattcttacctgaagaagatgtcagtcttcttctcttagaccaactgccctgatatgaggtttagaggttaaagaacatgtgtgtatttacatgatctttgtattctgccttttcgtccctcactaatgacagctgcaccccaaggaaatggagctgtggaagagagggtttgataagaaattaagtaaatattggatctaatccatcaccctccaggaagcctttattactcctaaaaatttcaaccaaattcattaaaggacaagaactccaccagagtaggccataaacattggcaaaattagttgtaatccatgactagatttaatgtccctttgttttattcccatatggttataatgctttgcttggcattaggggtattttaagttttcttctgcctagtaagtgaatttgtgtttataatacaataatcataaaatatcacattaatattttataactgtacagttataaaatattttataagtaatatttatattttataagtaatattttataactgtacagttaactctggcccaaggaaaagatagtctgatagatgctgcagccccattttagcaaatgtgacctcacaggcctgaatgccatcgctattccacatctacaggatagacggaaaggaaagaaataaaaaaataggtacctaacactggcaagaggatgatgactcatgttatttcacttaacctttttatcttttaacatgaaggactcatacaggttgataagaaaccagtgacataaacagaccaaaaaatgatcagatctttcaaattagcaaaaaaataatattttttaaacaatgggtgaaaatacagtgtaacagtaccaattatcaacatgtgttgagaaccagaaaaatgttctttttctttgatcagcaacactatttgggaaaatctatcctcagggcctagcctggggccctggcacacagtaggcactcaacgaatatttgctgaacacacaaatacttatgatattttaaaaaattggcaacaatctgatacctaacaatagagggattaaatattatggaactgttaaataagatgcttatgaataccatgcagtaagatgggcaatatttatgccataagctttaatgaaacaaatgggtattaaatgtatgataaggttataaattactttttaaaagattacagggaaaaaaattgaaagatatacactgaaatgttttttgctcacagtggtgacaaggtttctcagcactggcactgttgacgttttaggctgtatgtctttgctgtgggaggctggcctgtgcactgcagggtgtttggcagcactcttggcctctgcccctagatagcaatagcagtcctccctcaaccagcccaattttgacaaccaaaaatgtttccaggcatcaccagatgctccctgggtgagagtgatgaaatagtaggggattttccccttcttttcttattttctgtaattccattatattactttaataataaagaaaaaaacataaaaaataaacgaatgttattattctacgtcagtttggatgtttggactccattttggggttctttccattatatcacttggtctgctaaacattctacggtttggtaaggtgaagtgattcatgaaattttggttttatttttttcctgatactaaaaataaaacattctttcacttggaaatttggacacagaacaccaaaaaaaatccataatctcatctctctttttctgtcttttccttccttttttccctttaaaaacaataaagagtgaaacctacctgttctccctctaatttaattcctaaatataatcactgtcaatatcttggacatttcctgtgtctaaacacacacacacacttttttttttcagcaaaagtggatttctgctacatgtagtgttctgcaacttactttctatgtgtttacaaaatcagtacatgtacatatgctgaattcagtccttaatggtattatattttgtgaatataccaaaatttgtttaaccacttagacaatctaggatattctcagtttgctgttatgagcaatgctcttcctttacatatacagacatatatatatatatgtgtgtgtgtgtgtttttgttttagtaggatagatttctaggagagggtgaaaggtcttatgacatccgcatttacgattgtaataggaagtatcaaagtgccccctaaagaaaaaaatcctcccattagtgggtaagaaagcctatttgttcatatcttcacaaacactaaatattagaaatatttacaattgtggtcaagctcataagtgaaaatggtatttcatatcttatattttttattgtgagattgaacatctttcatatgtttacatgtcacctgtatttcttattctctgaactatatgttatgacctttcactttttttcctcatgggttatgtgtagtttgtatagttgtcttattgattgttaggagctatttatatattaggaacattaatctcctgtcttatatatacgtggcatcgattagttgatcatttgtgagttcatgtctgtatacaaagattggagaggcactaagagggaaaacttacctctttcttatcaaagtttgtaaatatatgtataacagaagagggagaaaatattaataaatgcacagattggctgaaatagagtataaatcttttactcccctacttcaacataaactgcaaaaggagagtgacttttctttcactctgacttccgtattcctcatgcttaaaatagtgcctagcacagaagaggtgctcaatcagtgtttgctaaacgaaataattagtcacatttcaagcaggatgactaaatgaagaatagaatctaggcagatactctggaagagtggctgtgagtcattcatatcttagtatgaattagtcaaatccaactctctccccttcccactccccactgttagtagaagaatctgtttattgagagaatagatttataatttagaataagtgagaggggcagaagaggagattttgaaggatggcacctgaaggaggactagcatggctgagacagtgaagtggaagccttgaatagctaaagggtaagatgaaagtatttagctgtagggggaaaaagcattgacaggttggaaaagtaaaagtcagattctccttgctctgaaattttgtacagggcaggttctactaggtatgttacaatgcagaaaaaacatgaaataattgagaggaatttggtgcaatattatcttcttggcttcttttgagtgggcagatttttttcacggcctgtaactataataaatttgaaacttctcatcttttagtaacttttttcacttaagtttatgtggctgtgggcaatggaatgaagatattgaacttccaattccctgttgggtttccacaattacaagtcaatcatgactggttattagaagactatttcagttagaaccaccaagtcccatattgtcatattgtatgtttaattattaagtgaagcagtcttcttttcgtgttttccataattagggcattccagaaagatgaggatatttgctgtctttatattcatgacctactggcatttgctgaacggtaagacaccaaatccttccattaggttctatattttaaatattttaaccatgagtttaaaactaaaatgatcatttaaaatgcatgcaattttcttatagagagaacattctattctttcttctactttacacaatggcaaagtcttctttctactttacgcaatgataaagttacctgtgtcattttgtaaaaatatagagaatatagacaaattgaaagacacaaaataatctattacccatttcccagggttaactactgaaaatatctggggaaatggcctgtatgtatacatttatttgtttgctttcaacaaggccaagatcctttgatctttcagtcttggttgctctgtgacatgcctttcctgatgaggatactttaaggaagaattgtaagatacatggaaaatgtcaggctaacacagtactggcatcaccctgtgctctttcctgaactccataccaatgtacttcttgccagaaaactgatcaaaagtttagggaagtaaaaagagatgactgttagaatctaccattccctctatgtaggaagcaaataggtgtcctgtcaaaggacattctggggatgtctacatgaaaccaactctccctggttgtaaggactccatctccatataatatttatacagtaatatatgtttataaattgtgggggcaacttgtttagctaattttattattctgctattgggacactgtgtctcagcatgagatatagtgtcccaaaacatatttcaagcccattggataaaatatgtgtttagcaagttcttaaatataatgataacataaccgaccagataaagtgatttataaacgctgtgccaattttgtaaatgtttcgaggaattttcccttttctgaagattgtccttctttctttttagcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatggtaagaattattatagatgagaggcctgatctttattgaaaacatattccaagtgttgaagacttttcattcttgtaagtccatacttattttcaaacagaacagcatagtctgttcattcattcattcaattcatgaattcattcacataattatccaatttcttgagcacctatttgatagtcactggaaatccagagacaaacaacacagagccatgttctacagtatgtacagttttccaaaaagaatttctagtctttacttttttattacaaatggaatacgtatacttgcaaataattcagatactgtggaagagatcaaatgaattgcaaaagtgtccctcctcccttcaccactatctcccatggcatgcagagagagtaaccattatttgtgtgtccctccagaaatttttttattcaactactatttttttattttattaggtccgtcagttttccttttttgagcctctctatatcaaatgcaaataaatatattcagaacaaaccccactgtaaggttcacattaaaaaagacttgaagtcaccctatgaagacaaaaaataatcacattaagtgtgaaagaacctattcttccagtacaggataagccatacttactgggcatatattcatcttgaaaatctatactgatgttgtcttggggaattgaaaaggaactaggagtgttagttcctcggtattgacccacagttatgttatcaggtcacttgagttcaaagttttgtgttggcactagctaagtaaaggaaaacacctctgctttcattgttgagtttcacagaattgagagctgaaaggatcccaggcaggagcagctaatccaaactcccacaaagaacaaaaatcccccagaggatcttctgttcttatatttcctgcaatggcgtccctgtcatatcccacaatggcctccctgccatttggatatcccttccatatcctgttgaaattactccctaatagtaagctgaaatctgcccctctagttgtagtcttgggattatttcatttacatgatgaccttttaatatttgactagaattaaatcatctccccttggtctttccattcctgggctaactaccatcaatctgagggctaacaatacaagtagaaaaagtatacatttgtcactgatcactgatcaattattaatcaatgatcactgataactataaactcaaaaacaaaatcatgtggggattaagagaaatgtatcagttttatgttgtatttctggtccctgatactggctcaggtaatgccactattgtcaagaagataccacttgtaaagtagatttaattttcattatattttaccatatgcttctccattcatgacatctcttgagatgttgtggtttatactttcagtttttctccagtccatccgcaaatatcaggcatctactgtgttccaagatattaaagaaatcatcatgacttagcctcatcaacagcattgctagatctgggatggaaaggaagagtataatcctggcagtcaggaagaaggcagcataaagtataagtttctgcttccaaaaaaggtctctcatcagcctgtagggagtgtgtagggaagggacagctgtccttgtagtagggaagggttttattcaggtcgtctgggctccataatatcccttgtgtatctgcagtctcctttgccatggatcaacacaataggaaatcttccggcactgatggtttttccaagggggagttcttcctggagcaaagcaaatgaccaaccaggtttgaggacctgatttgtttgacaattccattttgtattgtaaattacttaattggcattctactcccaatccatcttgtcatttgcatacagtggttttgggattgagttcagctataccaaaagtctgaaccttctgcacttagaacaaggcaaccaccaagcttcacttgcactgaggccgtgtctccaatggaaatgaggcagctggcttgcaggagcttcccaactcagggaagtagaactcctgagtcacctccatatgcaaatgatttcacagtaatgctgttgaacttcacttcccatcacagcaaatgtgtggtaacatagcttccccacaggagtttactcaccatggtattttaaaggtgaaacatttcaaaactgaaatttgaaagaatttagttttggattcactcaattatcactatcacttcgggtgttattgcacctttcttgtttgtgagtttaaatgccagactctcaggccactaactttcaattaaaagtgtttttctttaatcgctgaacctaacagcagggaaaacgaaatgttcattcagactttcagaaccttcaatgagattaggcagctgaaagatcaaagtgttgcatagttgtcccgataaagctatttggatcatatggaccaaatcgactgctgtcattccccaccaaccccatctctccccaaaattcccagccctgtttaagtgttctctgtagcatttatctctatctagtatattgtgtagcatatcatatcatacttttctgttttgtttattgtctctctcctcctagaatataaactccacaagcacaaagatttgggcctgttttataatattgttgcatccccagggcctgatatacagcagagtggtggtacgaaaagagcacacaaaaaaatatttgttgagtcaatgaatgaatgatttcctcaaataggattagcctaaaattttggaaacatgaacagatttggatatgtgaaaatttatttccagactgttcatcaggaactgttagcagcttctaaagggtacactggagcagcagtagtaaaaggaggaagaggagcagctctgctactgctactatcgagtactactacaattagcacttgcttattctgtgtgttaggccctgtactgaacactctgtctaaattagttcatttcctcctggaaatgactctagggggtaagtgcttcatcatgtaagatgagtatttttcacattttgttgtgtctgaaatctgagtgtgtctttcaatgatggaatctttgattccatgataagtggtattattcccattttaaggatgaggaaactgaggtccaaagaaattaagtaatttgcccaaattcacccagcctagaaaatgataaagctagttctaaacccaagcagattagctctgaagtctgggcccttaataaccactttttattgcctatatttgtacctctggtgtacgtatcaagttatatgttgacttcaaaactatcatgaccttttcttggttttgattgtccaacattagtatagtgttctgggtctgcaaaaattttgattactcatctcatctgtaaaacattttgaactcgtgtgtttgtgcatgcacatttgtgtgtaattataaaaattttactttctgttaatatataagttgtatcataagaaactgccgtttttgaagagcaaaaaaaggttgaatgttaccagttacatctggttcaacctaatagacatttgtacaaaaacagacattttaagaggttgaaataaaaatttaataaacaatattttcagtttttactaattgtgatgcttcactatcattagctaatatgtcaaggcataatataccttagggtgaactttatcattaacaaaggtggatggtgtcaataatcttgaggtttgtgtttttttatataacactgcgaggtctaattaagtacttactgtttaccacctcatacagtggccgataaaaagtgtcacttctgctgtttcctctgggttgtgcttgaattattagtattatcttcagtcctcagtttctttgtgggaaactttttaattagttgtttaattttgtaagatggttagtttagtcaaaattagataagagaatttgaaaatccgtagctaccccaaagcaacctacacataagaactattatttttgtgttttgaaatcataattttattgatttccagtgtttccactggtagtggtttcattgatataggagtatcaaaacatcactcattatttatttcagtttcatttgatcctagccgttttgtattaactctctgtgaagaaattacctcacaaatctattgctgtccttggtaaaggaatggagaattaaggctctagatcattagtggttacactatagtattagaagtaaaaaaaagattataccaacaaaataagaacatgttaatgtacttgtaatgaataaacatgaataaagctcttatgctatataggtgcactaaacaatctactagaattgtcagcaaactacgtatcttaatcctgaaagggtcccaaaccaatgatctaaaattgaatcaaactttcttccttgagcataattacttaaatgatttattaaaatagccagcatttaaaagcttaaaatgtaaatatcataatgtggtatcctagatagcatcccagaacagaaaaaggatattagggaaaaactggaggaatggaataaattatgcagtttagttattaataatgtactaacgtccttagttatgacgattgtaccatggtaatgtaagatactaacaatagaggaaaccgggtaaggagtatacagtaactctatactatctttgcaacttttttgtaaatttaaaacttctaaaataaagaacaaatttaaacattaaaaagtatcaccaggaacatatatcactgtttacagatgaaatactatgtattttcatatctaatttctgatcattgacttcaaatcagaaaagtgaatgacacctcaaaatcaggttttctgtttactgaagtctaagaaaagaaagcataccagctggagagattcatgtttataaagacagatttataacaacaaaaataaaatatccaagaataaatttaagaagaagcactttactgagaaacatatgaaaacctgaacaaatggagagggatattttgtatttgaatagaaagacttctggtttaaagataattctctttaaattattttttgtagaaatttaaggggtacaagagcagtgttgtcacatggatatattacatagtggtgaagtctggggttttagtgtaaattaatctttacattttgtttgagcccaataaatgtaccaacatgatttttatagaaagatagtcattcctattaatccaaacttgtcccaactttgaattgaattgaggcagagctagcaggtgttccccacggctgaggcatctgaacattaagcatatccctctgagaaccagcctgcattgatactctttctaatgtggacagcatcaagctatgtacgtagttctgtgctcagcaaaagccctgacttctttttgtttatgtcctagccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccaggtaatattctgaatgtgtccattaaaatatgtctaacactgtcccctagcacctagcatgatgtctgcctatcatagtcattcagtgattgttgaataaatgaatgaatgaataacactatgtttacaaaatatatcctaattcctcacctccattcatccaaaccatattgttacttaataaacattcagcagatatttatggaatataccttttgttccatgcattgtagtactcattggatacacatagaataataagactcagttcacactcttcaggaaacagataaaaaactaagaaacaaacaaaaaacaggcaatccaacaccatgtgggaaatgctttcatagccgggaaacctggggaatacctgagaggaatactcaattcaggccttgtttcaggaatccaaatcctggcacatcagagctgcttccctctttccagggtggcaggaaataaatggaacatatttttctatcttatgccaaacatgagggaccctttctccccggtgcctctcccaaggtagtctacaatatttcaactctagcagtctgcttagtgcatagaacatgaggctgtgtgtccctgggcaaattactagacttctgtgtgcttcactttccctgtaggattataatctactgagcaagcttattgtaagggtcagattagcaacagtgtatgaaaatgatttgagaccattgcctgcacaaattcaactatttttttttatctcactactctacagaagtaggtagggtgggagacagagtctgatgagaggctcagaatgtgaaagaaagtgaggcgagtgagcatgatatttaatataaacacaaagatattctgagaagagctgctcactgccccctcccccaatacatgttgataggaaaatgccacgtacttcagcaaaaacaactgaaaaattagatagaaaagtcaatcaataggaaaagataatccaggacggtgttgtgaacagaaagagggggaaaaaactttagaaaatgatggggatgctcttactggggtacgagtcctcaggtattgaactggctttcagtaaaagctagattagtgggttcctgccatttacaagctgttttatgacaacttacttgttgggtggcctacagtaactcacctaactgcactgagtctgtttcctcatctgtaaattggggatttttttttaaatacctggcatgcctaactcataaagttgttctgaaactgaaataaaacatacgtgaacaggcattgtaaactgtaagttacggaaaaagctggctgttgttgtgtctttaaagtttcacctgggtagtcaaagatggatcatgggtctcagtggagagctgagccaggcaggagctgactaagggtgagaggtgggagttagcagcctctgaacatctgtgtaccatgggaccccctttcctcctgcatggtaccccagacaaggagcctagtaagagatactaatggcttgttgtccagagatgttcaaactgcagagaaagataagacaacaagcattggcctccaatcatgatgacagataggaggaggtgggagctccttagcagtgctggttggccttccatgttctactgtgggccatctctgccatgtactgtaggctactagcttctatattaaagaatgcaagaggggccaggagcggaggctcatgcctgtaatctcagcactttgggaggccaaggtgggcagatcacttgaggtcaggagtttgtgaccagcctggccaacatggtgaaactctgcctttactaaaaatataaaaattagctgggtgtggtggtgtgcacctgtaatcccagctactcgggagactgaggcacaagaattgcttgaacctgggaggcggaagttgcagtgagcccagattgcgccactgcactccaccctgggcaacagagaaagactctgcctcaaaaaaaaaaaaaaaaagcaagaggaagtgaaataatcaaggccgccatttaatagtgagcagccactccatgtggtactgtgcaagcacattataaatattagcctcacaagaaatgtattagcatttgtattttgtacactggttaagtatcttgcccaagacctcaaaactggttaagggcagcagaatttagccccagcaccaccttttcaaagcctgggcttctcacacttctccatgctgttcccattttaacacaggtatctcgccattccagccactcaaactttggcatttaagaaaattatcctaaagctaaactaaacttcaaggatgaccattctcctgaccccttcccatcaaaattttatctttagtcagtttgttttcgttttgttttgtttttcagaactacctctggcacatcctccaaatgaaaggactcacttggtaattctgggagccatcttattatgccttggtgtagcactgacattcatcttccgtttaagaaaaggtagtatttccttaattgcagtggtctccactgggggtgaggaaggggtgagaattggatcatggctgcaaggaaacccgacttaacctctgcaaggtggtgcaaaggcattccactgttcaacagcaattatattgaagctgagtgggatcactgggtgaagatgaagcgtaaggggtgaggggcaggagaatgggtatggatggaggtagaagatgcagtgtcatacagtttttttctatcatgaaaataaccacagacttacagaagagaaagagctaaaatgcccgtcattttcagttgcattttagtcttgcattagttgcaaccagctggtttctgggtaccctaagtaataaaaatagttcctctgtagaactgtagtatgtttaccatagagtattttgcaaaatttttggtagaggatgttacataatttgcatgtgttcatttctccatttacctgtgggaacaattaaaatccaggaaaatgagtatattcaaataatttcctcccatttaagatgagtcagagtaaataattcctccaatacttagagaagtataccaagagatccagtgatggtatagagttgtctgatgttaaatagggaagtagaatatggaaggggattccaatagtcgttgaaaaattccccataaccccttacatgggggaaagtagtgttaactgagagagtagagataagctgtttccaaaaattatattcttaacaggactgagatagccagaatataaggatcaagtttcaatgacagtaagatcctgagatggagttgatttgcacaaagaaataattgttgccagcatgcattttgaatatttctctggaaaaaaagattagttggcagtagaaatggatagaaatcaatagatattaaaatacctcagaatttggttcatctctgggaaaagatgaaaaataaaagtgtatactcctcaagaacatctaggatcaaaagcatgtgccctacactattgaattaattaacctcataagttgggacctgtggaataaggatgtccaccagacttcctagggattacaaatgtttcacagaacttgaaatttaaacttgggtcactgtatgggatgtagagctgtgctatatggaaataaaaatgatttctttttctcaagggagaatgatggatgtgaaaaaatgtggcatccaagatacaaactcaaagaagcaaagtggtaagaatatcagaaggaattgggaagtaaaagtcaaaggaaacaaaaagctaaagcaataacaaagagaaatccatcagtcataatctcctctccttttaaagaatgctggttcccctttgcctcacagctaacacaagaactcctccaccgtctgaggaggtttaggagcagggaaggggaaggagtcagcttcatttgctaatcttctgttgccctgcaccctagcagctccttgcagcaggggacaaggatgacttaggtggatggataattaattgattctaaaatattgtgtgtcagtattgtaatactatgttaattgcaccatgcacggtatctcatttaatcccccaccccttgccattaccaaagagagagagagagagagagagagaaatactagaatttatcctcattttacagtagagaaaacagagggtcaagaagataatgtaaagtgcccaagaacacacagctgatcacaaaaatcaagcttgggggccattagcctaaccacagacccttactcttaacccatctgcttcaatccattttgctacaaatgtttacatttataagcagggcagaaaaacctcatccaggttattgaactaagaagaaagttatattaaggtttctaatttttttaatgtagttagaaaccaaacttaacaatgagcccaagtttaaagcagtctaattaacctggacaagctcaggcaagtttcattctgtggcccatagcatcatctgtgttgtaaagctaagtagcaaatgttgtttgggtcatgctgggggacaagccatcccaatttgctcaggactgaggggttttccaggatatcatgtaaggataattgggtacaaatataacctgctgctttctctcatttcaaatttatcatttatcatatcagcaactatgagttatgttttttattagatttcttgttactttttccccagaccacttcccatgaaattaatatactattatcactctccagatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagtta